(19) United States
(12) Patent Application Publication
(10) Pub. No.: US 2011/0152332 A1
(43) Pub. Date: Jun. 23, 2011

(54) Pesticidal Heterocyclic Compounds

(75) Inventors: Tetsuya Murata, Oyama-shi (JP); Mamoru Hatazawa, Tsukuba-shi (JP); Peter Brüchner, Tochigi ken (JP); Eiichi Shimojo, Omaya-shi (JP); Teruyuki Ichihara, Oyama-shi (JP); Masashi Ataka, Oyama-shi (JP); Katsuhiko Shibuya, Shimotsuke-shi (JP); Ulrich Görgens, Ratingen (DE)

(73) Assignee: Bayer CropScience AG, Monheim (DE)

(21) Appl. No.: 12/917,116

(22) Filed: Nov. 1, 2010

(30) Foreign Application Priority Data
Oct. 30, 2009 (JP) 2009-250744

(51) Int. Cl. A01N 43/76; C07D 263/24; C07C 215/30; C07C 205/26; C07C 317/22; C07C 323/19; A61K 31/421; A61P 33/00; A01P 5/00; A01P 7/04; A01P 7/02; A01P 3/00; A01P 1/00

(52) U.S. Cl. 514/376; 548/231; 548/232; 548/229; 564/363; 564/365; 568/812; 568/648; 568/30; 568/44

(57) ABSTRACT

The present invention relates to novel pesticidal azolidine derivatives as well as to oxazolidinone derivatives and their use as pesticides for combating animal parasites which occur in the agrochemical field and in the field of veterinary medicine, respectively.

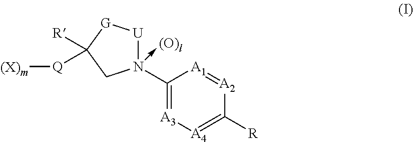

wherein X, m, R′, Q, G, U, l, A₁ to A₄ and R are as defined herein.

This application claims priority to Japanese Patent Application No. 2009-250744, filed Oct. 30, 2009, the disclosure of which is incorporated by reference herein in its entirety.

The present invention relates to novel pesticidal azolidine derivatives as well as to oxazolidinone derivatives and their use as pesticides for combating animal parasites which occur in the agrochemical field and in the field of veterinary medicine, respectively.

WO 2007/123853 and JP-A No. 2008-110971 describe certain 5-membered heterocyclic compounds and nitrogen-including heterocyclic compounds, respectively, which are considered being useful as pest controlling agents. In particular, JP-A-2008-110971 describes cyclic amine compounds of the following formula

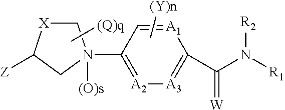

In addition, from WO 2008/128711 it is known that certain types of arylpyrrolidines have a pesticidal activity.

Since ecological and economic demands on modern plant treatment agents are continually increasing, particularly in respect to the amount applied, residue formation, selectivity, toxicity and favourable production methodology, and also because, for example, resistance problems can occur, there is the on-going task to develop new plant treatment agents that, at least in certain areas, are able to demonstrate advantages over known agents. It is particularly favorable to provide novel plant treatment agents which are safe when applied, particularly which are safe for beneficials, such as bees.

Inventors of the present invention extensively studied to obtain novel compounds which are pesticidal agents having an excellent effect and a broad spectrum. As a result, the inventors found novel azolidine derivatives, in particular novel oxazolidinone derivatives which avoid before mentioned pitfalls and which are particularly advantageous when applied. Also, they are effective against pests resistant to organophosphorus agents or carbamate agents.

Thus, the invention is directed to azolidine derivatives of formula (I)

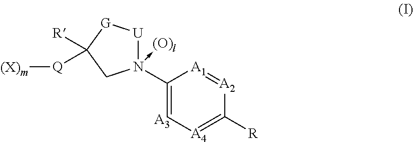

wherein
R′ represents C₁₋₁₂ alkyl or C₁₋₁₂ haloalkyl which may be substituted; preferably R′ represents C₁₋₆ alkyl or C₁₋₆ haloalkyl which may be substituted; more preferably R′ represents C₁₋₄ alkyl or C₁₋₄ haloalkyl; most preferably R′ represents CF_{3;}
l represents 0 or 1;
G represents CH, CH₂, N, O, S, C═O, C═S or NR¹;
U represents CH, CH₂, N, C═O, C═S, S═O, SO₂ or NR², under the proviso that, G and U do not simultaneously represent CH₂, and when G and U each represent CH or N, a bond between G and U is a double bond;
R¹ and R² each independently represent hydrogen, cyano, C₁₋₁₂ alkyl, C₃₋₈cycloalkyl, C₃₋₈ cycloalkyl-C₁₋₁₂ alkyl, C₂₋₆ alkenyl, C₂₋₆ alkynyl, C₁₋₁₂ alkoxy-carbonyl or C₁₋₁₂ thioalkoxy-carbonyl, wherein each group from the C₁₋₁₂ alkyl to the C₁₋₁₂ thioalkoxy-carbonyl described herein may be substituted; preferably R¹ and R² each independently represent hydrogen, cyano, C₁₋₆ alkyl, C₃₋₆ cycloalkyl, C₃₋₇ cycloalkyl-C₁₋₆ alkyl, C₂₋₄ alkenyl, C₂₋₄ alkynyl, C₁₋₆ alkoxy-carbonyl or C₁₋₆ thioalkoxy-carbonyl, wherein each group from the C₁₋₆ alkyl to the C₁₋₆ thioalkoxy-carbonyl described herein may be substituted; more preferably R¹ and R² each independently represent hydrogen, cyano, C₁₋₄ alkyl, C₃₋₆ cycloalkyl, C₃₋₆ cycloalkyl-C₁₋₄ alkyl, C₂₋₄ alkenyl, C₂₋₄ alkynyl, C₁₋₄ alkoxy-carbonyl or C₁₋₄ thioalkoxy-carbonyl, wherein each group of the said C₁₋₄ alkyl to C₁₋₄ thioalkoxy-carbonyl may be substituted;
R¹ and R² may form a 3-to 6-membered hydrocarbon ring with the carbon atom to which they are bound;
X which can be the same or different represents hydrogen, halogen, nitro, cyano, hydroxy, mercapto, SF₅, amino, C₁₋₁₂ alkyl, C₁₋₁₂ alkoxy, C₃₋₈ cycloalkyl, C₁₋₁₂ alkylthio, C₁₋₁₂ alkylsulfinyl, C₁₋₁₂ alkylsulfonyl, C₁₋₁₂ alkylsulfonyloxy, C₁₋₁₂ alkylaminosulfonyl, di(C₁₋₁₂ alkyl)amino-sulfonyl, C₁₋₁₂ alkylcarbonylamino, benzoylamino, tri(C₁₋₁₂ alkyl)silyl, or C₁₋₁₂ alkoxyimino, C₁₋₁₂ alkylsulfinylimino, C₁₋₁₂ alkylsulfonylimino, C₁₋₁₂ alkoxy-carbonyl, C₁₋₁₂ alkylcarbonyl, aminocarbonyl, C₁₋₁₂ alkylamino-carbonyl, amino-thiocarbonyl, C₁₋₁₂ alkylamino-thiocarbonyl, di(C₁₋₁₂ alkyl)amino-carbonyl or di(C₁₋₁₂ alkyl)amino-thiocarbonyl, wherein each group of the said C₁₋₁₂ alkyl to di(C₁₋₁₂ alkyl)amino-thiocarbonyl may be substituted; preferably X which can be the same or different represents hydrogen, halogen, nitro, cyano, hydroxy, mercapto, SF₅, amino, C₁₋₆ alkyl, C₃₋₇ cycloalkyl, C₁₋₆ alkoxy, C₁₋₆ alkylthio, C₁₋₆ alkylsulfinyl, C₁₋₆ alkylsulfonyl, C₁₋₆ alkylsulfonyloxy, C₁₋₆ alkylaminosulfonyl, di(C₁₋₆ alkyl)amino-sulfonyl, C₁₋₆ alkylcarbonylamino, benzoylamino tri(C₁₋₆ alkyl)silyl, C₃₋₆ alkoxyimino, C₁₋₆ alkylsulfinylimino, C₁₋₆ alkylsulfonylimino, C₁₋₆ alkoxy-carbonyl, C₁₋₆ alkylcarbonyl, aminocarbonyl, C₃₋₆ alkylamino-carbonyl, amino-thiocarbonyl, C₁₋₆ alkylamino-thiocarbonyl, di(C₁₋₆ alkyl)amino-carbonyl or di(C₁₋₆ alkyl)amino-thiocarbonyl, wherein each group of the said C₁₋₆ alkyl to di(C₁₋₆ alkyl)amino-thiocarbonyl may be substituted;
m represents 1, 2, 3 or 4; or
   the chemical grouping (X)ₘ-Q- stands for a 6-membered aromatic cyclic group having the following formula

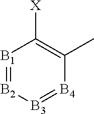

with B₁, B₂, B₃ and B₄ each independently representing C—X or N (nitrogen), under the proviso that only two of B₁, B₂, B₃ and B₄ may simultaneously represent N;
Q represents a 5-membered heterocyclic group which contains 1 to 4 heteroatoms selected from N, O and S;
   and wherein the chemical grouping

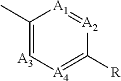

represents one of the following groups [g1] to [g6]

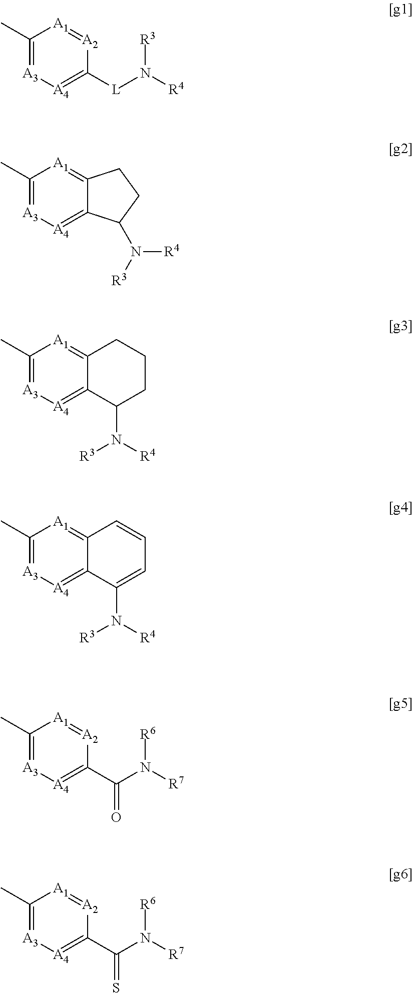

wherein
A₁, A₂, A₃ and A₄ each independently represent C—Y or N (nitrogen) under the proviso that only two of A₁, A₂, A₃ and A₄ may simultaneously represent N; when A₁ and A₂ represent C—Y, two Ys may form a benzene ring or a 5-to 6-membered heteroaromatic ring with the carbon atoms to which they are bound;
L represents a chemical group (CR¹R²)ₙ with
n being 1, 2 or 3;
R³ represents hydrogen, amino, hydroxy, cyano, C₁₋₁₂ alkyl, C₁₋₁₂ alkoxy, C₁₋₁₂ alkylcarbonylamino, C₁₋₁₂ alkylamino, C₃₋₈ cycloalkyl, C₂₋₁₂ alkenyl, C₂₋₁₂ alkynyl, C₁₋₁₂ alkylcarbonyl, CH₂—R⁵, C(═O)R⁵ or C(═S)R⁵, wherein each group from the C₁₋₁₂ alkyl to the C₁₋₁₂ alkylcarbonyl described herein may be substituted; preferably R³ represents hydrogen, amino, hydroxy, cyano, C₁₋₆ alkyl, C₁₋₆ alkoxy, C₁₋₆ alkylcarbonylamino, C₁₋₆ alkylamino, C₃₋₇ cycloalkyl, C₂₋₆ alkenyl, C₂₋₆ alkynyl, C₁₋₆ alkylcarbonyl, CH₂—R⁵, C(═O)R⁵ or C(═S)R⁵, wherein each group from the C₁₋₆ alkyl to the C₁₋₆ alkylcarbonyl described herein may be substituted;
R⁴ represents hydrogen, cyano, formyl, thioformyl, C₁₋₁₂ alkylcarbonyl, C₁₋₁₂ alkyl-thiocarbonyl, C₁₋₁₂ alkylamino-carbonyl, C₁₋₁₂ alkylamino-thiocarbonyl, C₂₋₂₄ (total carbon number) dialkylamino-carbonyl, C₂₋₂₄ (total carbon number) dialkylamino-thiocarbonyl, C₁₋₁₂ alkoxyamino-carbonyl, C₁₋₁₂ alkoxyamino-thiocarbonyl, C₁₋₁₂ alkoxy-carbonyl, C₁₋₁₂ alkoxy-C₁₋₁₂ alkylcarbonyl, C₁₋₁₂ thioalkoxy-C₁₋₁₂ alkylcarbonyl, C₁₋₁₂ alkylsulfenyl-C₁₋₁₂ alkylcarbonyl, C₁₋₁₂ alkylsulfonyl-C₁₋₁₂ alkylcarbonyl, C₁₋₁₂ alkoxy-thiocarbonyl, C₁₋₁₂ thioalkoxy-carbonyl, C₁₋₁₂ thioalkoxy-thiocarbonyl, C₁₋₁₂ alkylsulfonyl, C₃₋₇ cycloalkylcarbonyl, C₃₋₈ cycloalkyl-C₁₋₁₂ alkylcarbonyl, C₂₋₁₂ alkenyl-carbonyl, C₂₋₁₂ alkynyl-carbonyl, C₃₋₈ cycloalkylamino-carbonyl, C₂₋₁₂ alkenylamino-carbonyl, C₂₋₁₂ alkynylamino-carbonyl, C(═O)R⁵ or C(═S)R⁵, wherein each group from the C₁₋₁₂ alkylcarbonyl to the C₂₋₁₂ alkynylamino-carbonyl described herein may be substituted; preferably R⁴ represents hydrogen, cyano, formyl, thioformyl, C₁₋₆ alkylcarbonyl, C₁₋₆ alkyl-thiocarbonyl, C₁₋₆ alkylamino-carbonyl, C₁₋₆ alkylamino-thiocarbonyl, C₂₋₁₂ (total carbon number) dialkylamino-carbonyl, C₂₋₁₂ (total carbon number) dialkylamino-thiocarbonyl, C₁₋₆ alkoxyamino-carbonyl, C₁₋₆ alkoxyamino-thiocarbonyl, C₁₋₆ alkoxy-carbonyl, C₁₋₆ alkoxy-C₁₋₆ alkylcarbonyl, C₁₋₆ thioalkoxy-C₁₋₆ alkylcarbonyl, C₁₋₆ alkylsulfenyl-C₁₋₆ alkylcarbonyl, C₁₋₆ alkylsulfonyl-C₁₋₆ alkylcarbonyl, C₁₋₆ alkoxy-thiocarbonyl, C₁₋₆ thioalkoxy-carbonyl, C₁₋₆ thioalkoxy-thiocarbonyl, C₁₋₆ alkylsulfonyl, C₃₋₇ cycloalkylcarbonyl, C₃₋₇ cycloalkyl-C₁₋₆ alkylcarbonyl, C₂₋₄ alkenyl-carbonyl, C₂₋₄ alkynyl-carbonyl, C₃₋₇ cycloalkylamino-carbonyl, C₂₋₄ alkenylamino-carbonyl, C₂₋₄ alkynylamino-carbonyl, C(═O)R⁵ or C(═S)R⁵, wherein each group from the C₁₋₆ alkylcarbonyl to the C₂₋₄ alkynylamino-carbonyl described herein may be substituted; or
R³ and R⁴ may form a 3-to 6-membered heterocycle with the nitrogen atom to which they are bound, wherein the heterocycle may be substituted with X, keto, thioketo or nitroimino;
R⁵ represents phenyl which may be substituted or a 5-to 6-membered heterocyclic group which may be substituted and contains at least one heteroatom selected from N, O and S;
R⁶ and R⁷ each independently represent hydrogen, cyano, C₁₋₁₂ alkyl, C₃₋₈ cycloalkyl, C₂₋₆ alkenyl, C₂₋₆ alkynyl, C₁₋₁₂ alkoxy-carbonyl, C₁₋₁₂ alkylsulfonyl, C₆₋₁₀ aryl, C₇₋₉ aralkyl, a 5-to 6-membered heterocyclic group which contains at least one heteroatom selected from N, O and S, or C₁₋₁₂ alkyl-O—N═CH—, wherein each group from the C₁₋₁₂ alkyl to the C₁₋₁₂ alkyl-O—N═CH— described herein may be substituted; preferably R⁶ and R⁷ each independently represent hydrogen, cyano, C₁₋₆ alkyl, C₃₋₆cycloalkyl, C₂₋₄ alkenyl, C₂₋₄ alkynyl, C₁₋₆ alkoxy-carbonyl, C₃₋₆ alkylsulfonyl, C₆₋₁₀ aryl, C₇₋₉ aralkyl, a 5-to 6-membered heterocyclic group which contains at least one heteroatom selected from N, O and S, or C₁₋₆ alkyl-O—N═CH—, wherein each group from the C₁₋₆ alkyl to the C₁₋₆ alkyl-O—N═CH— described herein may be substituted; or
R⁶ and R⁷ together may form C₂₋₆ alkylene; preferably R⁶ and R⁷ together may form C₄₋₅ alkylene;
Y which can be the same or different represents hydrogen, halogen, nitro, cyano, hydroxy, mercapto, amino, C₁₋₁₂ alkyl, C₃₋₈ cycloalkyl, C₁₋₁₂ alkoxy, C₁₋₁₂ alkylthio, C₁₋₁₂ alkylsulfinyl, C₁₋₁₂ alkylsulfonyl, C₁₋₁₂ alkylsulfonyloxy, C₁₋₁₂ alkylaminosulfonyl, C₂₋₂₄ (total carbon number) dialkylamino-sulfonyl, C₁₋₆ alkylcarbonylamino, benzoylamino, tri-C₁₋₁₂ alkylsilyl, C₁₋₁₂ alkoxyimino, C₁₋₁₂ alkylsulfinylimino, C₁₋₁₂ alkylsulfonylimino, C₁₋₁₂ alkoxy-carbonyl, C₁₋₁₂ alkylcarbonyl, aminocarbonyl, C₁₋₁₂ alkylamino-carbonyl, amino-thiocarbonyl, C₁₋₁₂ alkylamino-thiocarbonyl, C₂₋₂₄ (total carbon number) dialkylamino-carbonyl or C₂₋₂₄ (total carbon number) dialkylamino-thiocarbonyl, wherein each group from the amino to the C₂₋₂₄ (total carbon number) dialkylamino-thiocarbonyl described herein may be substituted; preferably Y which can be the same or different represents hydrogen, halogen, nitro, cyano, hydroxy, mercapto, amino, C₁₋₆ alkyl, C₃₋₆ cycloalkyl, C₁₋₆ alkoxy, C₁₋₆ alkylthio, C₁₋₆, alkylsulfinyl, C₁₋₆ alkylsulfonyl, C₁₋₆ alkylsulfonyloxy, C₁₋₆ alkylaminosulfonyl, C₂₋₁₂ (total carbon number) dialkylamino-sulfonyl, C₁₋₆ alkylcarbonylamino, benzoylamino, tri-C₁₋₆ alkylsilyl, C₁₋₆ alkoxyimino, C₁₋₆ alkylsulfinylimino, C₁₋₆ alkylsulfonylimino, C₁₋₆ alkoxy-carbonyl, C₁₋₆ alkylcarbonyl, aminocarbonyl, C₁₋₆ alkylamino-carbonyl, amino-thiocarbonyl, C₁₋₆ alkylamino-thiocarbonyl, C₂₋₁₂ (total carbon number) dialkylamino-carbonyl or C₂₋₁₂ (total carbon number) dialkylamino-thiocarbonyl, wherein each group from the amino to the C₂₋₁₂ (total carbon number) dialkylamino-thiocarbonyl described herein may be substituted.

Compounds of formula (I) as defined herein are preferred wherein the ring moiety

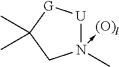

in formula (I) stands for one of the following moieties [m1] to [m16]

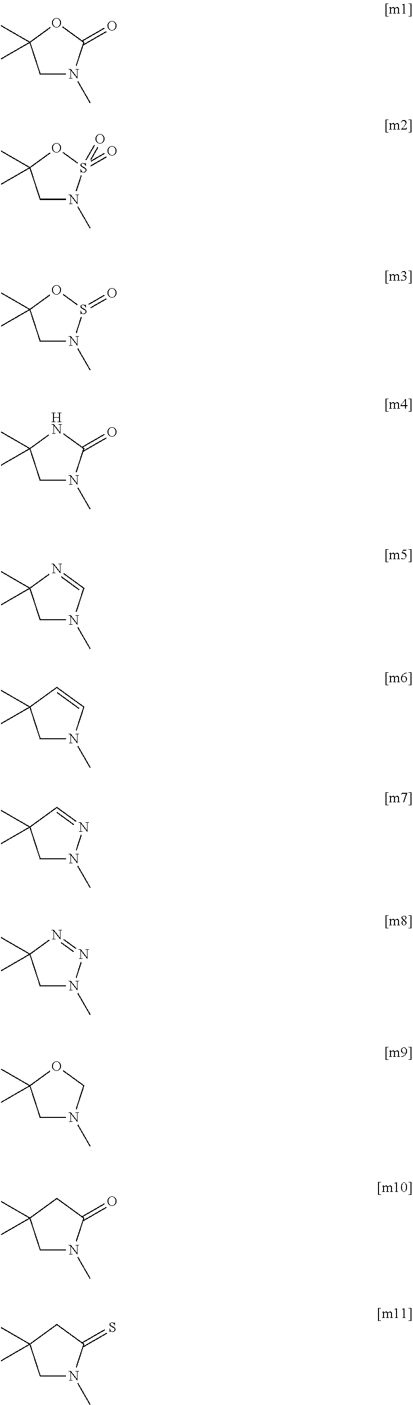

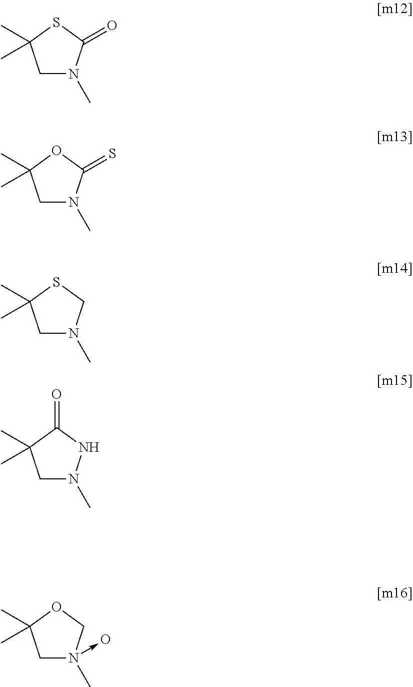

Among the moieties [m1] is preferred.

In the following embodiments, the insertion of an index to X and Y, which results in substituents X¹ to X⁵ and Y¹ to Y⁴ has been made to differentiate the substituents X and Y with respect to the binding site at the cycle. It is understood that in each case X¹ to X⁵ and Y¹ to Y⁴ are defined as given for X and Y, respectively. In cases in which the definitions of X¹ to X⁵ and Y¹ to Y⁴ are given as “as defined herein”, it is understood that X¹ to X⁵ are as defined herein for X (including the preferred, more preferred and most preferred definitions), and that Y¹ to Y⁴ are as defined herein for Y (including the preferred, more preferred and most preferred definitions).

In an embodiment (Group 1), the invention is directed to oxazolidinone derivatives of formula (I-I)

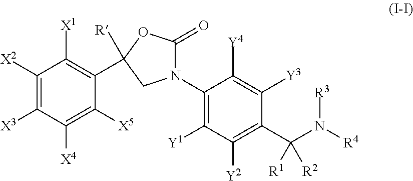

R′ represents C₁₋₁₂ alkyl or C₁₋₁₂ haloalkyl; preferably R′ represents C₁₋₆ alkyl, or C₁₋₆ haloalkyl; more preferably R′ represents C₁₋₄ alkyl, or C₁₋₄ haloalkyl; most preferably R′ represents CF₃;)
X¹, X², X³, X⁴, X⁵, Y¹, Y², Y³ and Y⁴ each independently represent hydrogen, halogen, nitro, cyano, hydroxy, mercapto, amino, SF₅, C₁₋₁₂ alkyl, C₃₋₈ cycloalkyl, C₁₋₁₂ alkoxy, C₁₋₁₂ alkylthio, C₁₋₁₂ alkylsulfinyl, C₁₋₁₂ alkylsulfonyl, C₁₋₁₂ alkylsulfonyloxy, C₁₋₁₂ alkylaminosulfonyl, di(C₁₋₁₂ alkyl)amino-sulfonyl, C₁₋₁₂ alkyl-carbonylamino, benzoylamino, tri(C₁₋₁₂ alkyl)silyl, C₁₋₁₂ alkoxyimino, C₁₋₁₂ alkylsulfinylimino, C₁₋₁₂ alkylsulfonylimino, C₁₋₁₂ alkoxy-carbonyl, C₁₋₁₂ alkyl-carbonyl, aminocarbonyl, C₁₋₁₂ alkylamino-carbonyl, aminothiocarbonyl, C₁₋₁₂ alkylamino-thiocarbonyl, di(C₁₋₁₂ alkyl)amino-carbonyl or di(C₁₋₁₂ alkyl)amino-thiocarbonyl, wherein each group of the said C₁₋₁₂ alkyl to di(C₁₋₁₂ alkyl)amino-thiocarbonyl may be substituted with halogen; preferably X¹, X², X³, X⁴, X⁵, Y¹, Y², Y³ and Y⁴ each independently represent hydrogen, halogen, nitro, cyano, hydroxy, mercapto, amino, SF₅, C₁₋₆ alkyl, C₃₋₇ cycloalkyl, C₁₋₆ alkoxy, C₁₋₆ alkylthio, C₁₋₆ alkylsulfinyl, C₁₋₆ alkylsulfonyl, C₁₋₆ alkylsulfonyloxy, C₁₋₆ alkylaminosulfonyl, di(C₁₋₆ alkyl)amino-sulfonyl, C₁₋₆ alkyl-carbonylamino, benzoylamino, tri(C₁₋₆ alkyl)silyl, C₁₋₆ alkoxyimino, C₁₋₆ alkylsulfinylimino, C₁₋₆ alkylsulfonylimino, C₁₋₆ alkoxy-carbonyl, C₁₋₆ alkyl-carbonyl, aminocarbonyl, C₁₋₆ alkylamino-carbonyl, aminothiocarbonyl, C₁₋₆ alkylamino-thiocarbonyl, di(C₁₋₆ alkyl)amino-carbonyl or di(C₁₋₆ alkyl)amino-thiocarbonyl, wherein each group of the said C₁₋₆ alkyl to di(C₁₋₆ alkyl)amino-thiocarbonyl may be substituted with halogen; more preferably X¹, X², X³, X⁴, X⁵, Y¹, Y², Y³ and Y⁴ each independently represent hydrogen, halogen, nitro, cyano, hydroxy, mercapto, amino, SF₅, C₁₋₄ alkyl, C₃₋₆ cycloalkyl, C₁₋₄ alkoxy, C₁₋₄ alkylthio, C₁₋₄ alkylsulfinyl, C₁₋₄ alkylsulfonyl, C₁₋₄ alkylsulfonyloxy, C₁₋₄ alkylaminosulfonyl, di(C₁₋₄ alkyl)amino-sulfonyl, C₁₋₄ alkyl-carbonylamino, benzoylamino, tri(C₁₋₄ alkyl)silyl, C₁₋₄ alkoxyimino, C₁₋₄ alkylsulfinylimino, C₁₋₄ alkylsulfonylimino, C₁₋₄ alkoxy-carbonyl, C₁₋₄ alkyl-carbonyl, aminocarbonyl, C₁₋₄ alkylamino-carbonyl, aminothiocarbonyl, C₁₋₄ alkylamino-thiocarbonyl, di(C₁₋₄ alkyl)amino-carbonyl or di(C₁₋₄ alkyl)amino-thiocarbonyl, wherein each group of the said C₁₋₄ alkyl to di(C₁₋₄ alkyl)amino-thiocarbonyl may be substituted with halogen; most preferably X¹, X⁵, Y¹, Y² and Y⁴ represent hydrogen, X², X³ and X⁴ each independently represent hydrogen, halogen or C₁₋₄ haloalkyl, and more preferably hydrogen, chloro, bromo or CF₃, Y³ represents halogen, C₁₋₄ alkyl or C₁₋₄ haloalkyl, and more preferably chloro, bromo, methyl, or CF₃;
R¹ and R² each independently represent hydrogen, cyano, C₁₋₁₂ alkyl, C₃₋₈ cycloalkyl, C₃₋₈ cycloalkyl-C₁₋₁₂ alkyl, C₂₋₁₂ alkenyl, C₂₋₁₂ alkynyl, C₁₋₁₂ alkoxy-carbonyl or C₁₋₁₂ thioalkoxy-carbonyl, wherein each group of the said C₁₋₁₂ alkyl to C₁₋₁₂ thioalkoxy-carbonyl may be substituted with halogen; preferably R¹ and R² each independently represent hydrogen, cyano, C₁₋₆ alkyl, C₃₋₇ cycloalkyl, C₃₋₇ cycloalkyl-C₁₋₆ alkyl, C₂₋₆ alkenyl, C₂₋₆ alkynyl, C₁₋₆ alkoxy-carbonyl, or C₁₋₆ thioalkoxy-carbonyl, wherein each group of the said C₁₋₆ alkyl to C₁₋₆ thioalkoxy-carbonyl may be substituted with halogen; more preferably R¹ and R² each independently represent hydrogen, cyano, C₁₋₄ alkyl, C₃₋₆ cycloalkyl, C₃₋₆ cycloalkyl-C₁₋₄ alkyl, C₂₋₄ alkenyl, C₂₋₄ alkynyl, C₁₋₄ alkoxy-carbonyl or C₁₋₄ thioalkoxy-carbonyl, wherein each group of the said C₁₋₄ alkyl to C₁₋₄ thioalkoxy-carbonyl may be substituted with halogen; most preferably R¹ represents hydrogen, R² represents hydrogen or C₁₋₄ alkyl, and more preferably hydrogen or methyl;
R³ represents hydrogen, amino, hydroxy, cyano, C₁₋₁₂ alkyl, C₁₋₁₂ alkoxy, C₁₋₁₂ alkyl-carbonylamino, C₁₋₁₂ alkylamino, C₃₋₈ cycloalkyl, C₂₋₁₂ alkenyl, C₂₋₁₂ alkynyl, C₁₋₁₂ alkyl-carbonyl, —CH₂—R⁵, —C(═O)R⁵ or —C(═S)R⁵, wherein each group of the said C₁₋₁₂ alkyl to C₁₋₁₂ alkyl-carbonyl may be substituted with halogen; preferably R³ represents hydrogen, amino, hydroxy, cyano, C₁₋₆ alkyl, C₁₋₆ alkoxy, C₁₋₆ alkyl-carbonylamino, C₁₋₆ alkylamino, C₃₋₇ cycloalkyl, C₂₋₆ alkenyl, C₂₋₆ alkynyl, C₁₋₆ alkyl-carbonyl, —CH₂—R⁵, —C(═O)R⁵ or —C(═S)R⁵, wherein each group of the said C₁₋₆ alkyl to C₁₋₆ alkyl-carbonyl may be substituted with halogen; more preferably R³ represents hydrogen, amino, hydroxy, cyano, C₁₋₄ alkyl, C₁₋₄ alkoxy, C₁₋₄ alkyl-carbonylamino, C₁₋₄ alkylamino, C₃₋₆ cycloalkyl, C₂₋₄ alkenyl, C₂₋₄ alkynyl, C₁₋₄ alkyl-carbonyl, —CH₂—R⁵, —C(═O)R⁵ or —C(═S)R⁵, wherein each group of the said C₁₋₄ alkyl to C₁₋₄ alkyl-carbonyl may be substituted with halogen; most preferably R³ represents hydrogen or C₁₋₄ alkyl;
R⁴ represents hydrogen, cyano, formyl, thioformyl, C₁₋₁₂ alkyl-carbonyl, C₁₋₁₂ alkyl-thiocarbonyl, C₁₋₁₂ alkylamino-carbonyl, C₁₋₁₂ alkylamino-thiocarbonyl, di(C₁₋₁₂ alkyl)amino-carbonyl, di(C₁₋₁₂ alkyl)amino-thiocarbonyl, C₁₋₁₂ alkoxyamino-carbonyl, C₁₋₁₂ alkoxyamino-thiocarbonyl, C₁₋₁₂ alkoxy-carbonyl, C₁₋₁₂ alkoxy-C₁₋₁₂ alkyl-carbonyl, C₁₋₁₂ thioalkoxy-C₁₋₁₂ alkyl-carbonyl, C₁₋₁₂ alkylsulfenyl-C₁₋₁₂ alkyl-carbonyl, C₁₋₁₂ alkylsulfonyl-C₁₋₁₂ alkyl-carbonyl, C₁₋₁₂ alkoxy-thiocarbonyl, C₁₋₁₂ thioalkoxy-carbonyl, C₁₋₁₂ thioalkoxy-thiocarbonyl, C₁₋₁₂ alkylsulfonyl, C₃₋₈ cycloalkyl-carbonyl, C₃₋₈ cycloalkyl-C₁₋₁₂ alkyl-carbonyl, C₂₋₁₂ alkenyl-carbonyl, C₂₋₁₂ alkynyl-carbonyl, C₃₋₈ cycloalkylamino-carbonyl, C₂₋₁₂ alkenylamino-carbonyl, C₂₋₁₂ alkynylamino-carbonyl, —C(═O)R⁵ or —C(═S)R⁵, wherein each group of the said C₁₋₁₂ alkyl-carbonyl to C₂₋₁₂ alkynylamino-carbonyl may be substituted with halogen; preferably R⁴ represents hydrogen, cyano, formyl, thioformyl, C₁₋₆ alkyl-carbonyl, C₁₋₆ alkyl-thiocarbonyl, C₁₋₆ alkylamino-carbonyl, C₁₋₆ alkylamino-thiocarbonyl, di(C₁₋₆ alkyl)amino-carbonyl, di(C₁₋₆ alkyl)amino-thiocarbonyl, C₁₋₆ alkoxyamino-carbonyl, C₁₋₆ alkoxyamino-thiocarbonyl, C₁₋₆ alkoxy-carbonyl, C₁₋₆ alkoxy-C₁₋₆ alkyl-carbonyl, C₁₋₆ thioalkoxy-C₁₋₆ alkyl-carbonyl, C₁₋₆ alkylsulfenyl-C₁₋₄ alkyl-carbonyl, C₁₋₆ alkylsulfonyl-C₁₋₆ alkyl-carbonyl, C₁₋₆ alkoxy-thiocarbonyl, C₁₋₆ thioalkoxy-carbonyl, C₁₋₆ thioalkoxy-thiocarbonyl, C₁₋₆ alkylsulfonyl, C₃₋₇ cycloalkyl-carbonyl, C₃₋₇ cycloalkyl-C₁₋₆ alkyl-carbonyl, C₂₋₆ alkenyl-carbonyl, C₂₋₆ alkynyl-carbonyl, C₃₋₇ cycloalkylamino-carbonyl, C₂₋₆ alkenylamino-carbonyl, C₂₋₆ alkynylamino-carbonyl, —C(═O)R⁵ or —C(═S)R⁵, wherein each group of the said C₁₋₆ alkyl-carbonyl to C₂₋₆ alkynylamino-carbonyl may be substituted with halogen; more preferably R⁴ represents hydrogen, cyano, formyl, thioformyl, C₁₋₄ alkyl-carbonyl, C₁₋₄ alkyl-thiocarbonyl, C₁₋₄ alkylamino-carbonyl, C₁₋₄ alkylamino-thiocarbonyl, di(C₁₋₄ alkyl)amino-carbonyl, di(C₁₋₄ alkyl)amino-thiocarbonyl, C₁₋₄ alkoxyamino-carbonyl, C₁₋₄ alkoxyamino-thiocarbonyl, C₁₋₄ alkoxy-carbonyl, C₁₋₄ alkoxy-C₁₋₄ alkyl-carbonyl, C₁₋₄ thioalkoxy-C₁₋₄ alkyl-carbonyl, C₁₋₄ alkylsulfenyl-C₁₋₄ alkyl-carbonyl, C₁₋₄ alkylsulfonyl-C₁₋₄ alkyl-carbonyl, C₁₋₄ alkoxy-thiocarbonyl, C₁₋₄ thioalkoxy-carbonyl, C₁₋₄ thioalkoxy-thiocarbonyl, C₁₋₄ alkylsulfonyl, C₃₋₆ cycloalkyl-carbonyl, C₃₋₆ cycloalkyl-C₁₋₄ alkyl-carbonyl, C₂₋₄ alkenyl-carbonyl, C₂₋₄ alkynyl-carbonyl, C₃₋₆ cycloalkylamino-carbonyl, C₂₋₄ alkenylamino-carbonyl, C₂₋₄ alkynylamino-carbonyl, —C(═O)R⁵ or —C(═S)R⁵, wherein each group of the said C₁₋₄ alkyl-carbonyl to C₂₋₄ alkynylamino-carbonyl may be substituted with halogen; most preferably R⁴ represents hydrogen, C₁₋₄ alkyl-carbonyl, C₁₋₄ haloalkyl-carbonyl, C₃₋₆ cycloalkyl-carbonyl, C₁₋₄ alkoxy-C₁₋₄ alkyl-carbonyl, C₁₋₄ thioalkoxy-C₁₋₄ alkyl-carbonyl, C₁₋₄ alkylsulfenyl-C₁₋₄ alkyl-carbonyl, C₁₋₄ alkylsulfonyl-C₁₋₄ alkyl-carbonyl or C₁₋₄ alkylamino-carbonyl, and among the most preferred groups, C₁₋₄ alkyl-carbonyl, C₁₋₄ haloalkyl-carbonyl or C₃₋₆ cycloalkyl-carbonyl are more preferred; and
R⁵ is phenyl which may be substituted, or a 5-or 6-membered heterocycle which contains at least one heteroatom that may be selected from N, O and S and which may be substituted.

Among before mentioned oxazolidine compounds, oxazolidinone derivatives of formula (I-Ia) are preferred

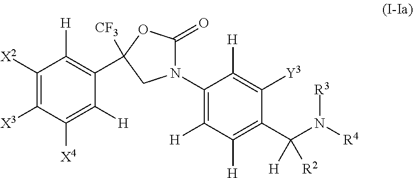

wherein
X², X³ and X⁴ each independently represent hydrogen, halogen or C₁₋₄ haloalkyl; more preferably independently represent hydrogen, chloro, bromo or CF₃;
Y³ represents halogen, C₁₋₄ alkyl or C₁₋₄ haloalkyl; more preferably represent chloro, bromo, methyl or CF₃;
R² represents hydrogen or C₁₋₄ alkyl; more preferably represents hydrogen or methyl;
R³ represents hydrogen or C₁₋₄ alkyl; and
R⁴ represents hydrogen, C₁₋₄ alkyl-carbonyl, C₁₋₄ haloalkyl-carbonyl, C₃₋₆ cycloalkyl-carbonyl, C₁₋₄ alkoxy-C₁₋₄ alkyl-carbonyl, C₁₋₄ thioalkoxy-C₁₋₄ alkyl-carbonyl, C₁₋₄ alkylsulfenyl-C₁₋₄ alkyl-carbonyl, C₁₋₄ alkylsulfonyl-C₁₋₄ alkyl-carbonyl or C₁₋₄ alkylamino-carbonyl, and more preferably C₁₋₄ alkyl-carbonyl, C₁₋₄ haloalkyl-carbonyl or C₃₋₆ cycloalkyl-carbonyl.

In another embodiment (Group 2), the invention is directed to oxazolidinone derivatives of formula (I-II)

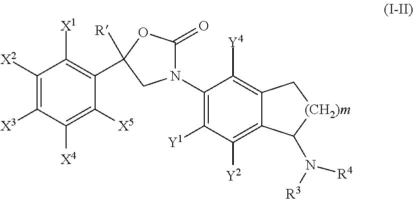

wherein X¹, X², X³, X⁴, X⁵, Y¹, Y², Y⁴, R′, R³ and R⁴ are as defined herein and m represents 1 or 2.

Since one or more asymmetric carbons may be contained in the compounds according to the present invention, the compounds of the present invention may include optical isomers.

The compounds according to the invention exhibit a potent activity as agents for controlling harmful pests such as insects, acari, and/or microorganisms. The compounds according to the present invention have a strong pesticidal activity.

If not defined otherwise, the term “alkyl” stands for linear or branched C₁₋₁₂ alkyl, preferably C₁₋₆ alkyl, and more preferably C₁₋₄ alkyl, for example, methyl, ethyl, n-or iso-propyl, n-, iso-, sec-or tert-butyl, n-pentyl, n-hexyl, n-heptyl, n-octyl, n-nonyl, n-decyl, n-undecyl, n-dodecyl and the like.

Further, for each alkyl moiety contained in the group having the alkyl as a part, those described above for the “alkyl” can be also exemplified.

“Acylamino” represents alkylcarbonylamino, cyclopropylcarbonylamino and benzoylamino, for example. As for the alkyl moiety, those described herein for “alkyl” can be also exemplified

If not defined otherwise, the term “haloalkyl” or “alkyl substituted with halogen” stands for a hydrocarbon group (chain) in which at least one hydrogen atom on linear or branched C₁₋₁₂ alkyl, preferably C₁₋₆ alkyl, and more preferably C₁₋₄ alkyl is substituted with halogen, or stands for a perfluoroalkyl in which all substitutable hydrogens on the alkyl are substituted with fluorines.

Examples thereof include CH₂F, CHF₂, CF₃, CF₂Cl, CFCl₂, CF₂Br, CF₂CF₃, CFHCF₃, CH₂CF₃, CFClCF₃, CCl₂CF₃, CF₂CH₃, CF₂CH₂F, CF₂CHF₂, CF₂CF₂Cl, CF₂CF₂Br, CFHCH₃, CFHCHF₂, CFHCHF₂, CHFCF₃, CHFCF₂Cl, CHFCF₂Br, CFClCF₃, CCl₂CF₃, CF₂CF₂CF₃, CH₂CF₂CF₃, CF₂CH₂CF₃, CF₂CF₂CH₃, CHFCF₂CF₃, CF₂CHFCF₃, CF₂CF₂CHF₂, CF₂CF₂CH₂F, CF₂CF₂CF₂Cl, CF₂CF₂CF₂Br, CH(CHF₂)CF₃, CH(CF₃)CF₃, CF(CF₃)CF₃, CF(CF₃)CF₂Br, CF₂CF₂CF₂CF₃, CH(CF₃)CF₂CF₃ or CF(CF₃)CF₂CF₃. Haloalkyl may be further substituted.

If not defined otherwise, the term “alkoxy” stands for linear or branched C₁₋₁₂ alkoxy, preferably C₁₋₆ alkoxy, and more preferably C₁₋₄ alkoxy, for example, methoxy, ethoxy, n-propoxy, i-propoxy, n-, iso-, sec-or tert-butoxy, pentyloxy, or hexyloxy. The alkoxy may be further substituted with halogen or other substituents.

If not defined otherwise, the term “halogen” and each halogen moiety contained in the group substituted with halogen stands for fluorine, chlorine, bromine or iodine, and preferably fluorine, chlorine or bromine.

If not defined otherwise, the term “cycloalkyl” stands for C₃₋₈ cycloalkyl such as cyclopropyl, cyclobutyl, cyclopentyl, cyclohexyl, cycloheptyl, and cyclooctyl. Preferably, it represents C₃₋₇ cycloalkyl and more preferably C₃₋₆ cycloalkyl.

If not defined otherwise, the term “cycloalkyl” indicates C₃₋₈ cycloalkyl, preferably C₃₋₇ cycloalkyl, and more preferably C₃₋₆ cycloalkyl, for example, cyclopropyl, cyclobutyl, cyclopentyl, cyclohexyl, cycloheptyl, and cyclooctyl. Further, for each cycloalkyl moiety contained in the group having the cycloalkyl as a part, those described above for the “cycloalkyl” can be also exemplified. Cycloalkyl substituted with halogen means halocycloalkyl, and examples thereof include fluorocyclopropyl, chlorocyclopropyl, difluorocyclopropyl, dichlorocyclopropyl, and undecafluorocyclohexyl.

If not defined otherwise, the term “Aralkyl” represents arylalkyl. For example, it represents benzyl and phenethyl.

If not defined otherwise, the term “Aryl” represents a C₆₋₁₂ aromatic hydrocarbon group. For example, it represents phenyl, naphthyl, or biphenyl, preferably a C₆₋₁₀ aromatic hydrocarbon group, and more preferably a C₆ aromatic hydrocarbon group, or phenyl.

If not defined otherwise, the term “alkenyl” stands for C₂₋₁₂ alkenyl, preferably C₂₋₆ alkenyl, for example, vinyl, allyl, 1-propenyl, 1-(or 2-, or 3-)butenyl, 1-pentenyl, and more preferably it represents C₂₋₄ alkenyl.

If not defined otherwise, the term “alkynyl” stands for C₂₋₁₂ alkynyl, preferably C₂₋₆ alkynyl such as ethynyl, propargyl, 1-propynyl, butan-3-ynyl, and pentan-4-ynyl, and more preferably stands for C₂₋₄ alkynyl.

If not defined otherwise, the term “heterocycle” stands for a 5-or 6-membered heterocyclic group which contains at least one of heteroatom selected from N, O and S, and also a fused heterocyclic group of which ring can be benzo-fused. The carbon atom of the heterocycle may be substituted with oxo or thioxo. Specific examples of the heterocycle include pyrrolidinyl, piperidinyl, morpholinyl, thiomorpholinyl (saturated heterocycles), dihydropyrrolyl, dihydroisoxazolyl, dihydropyrazolyl, dihydrooxazolyl, dihydrothiazolyl (partially saturated heterocycles), and furyl, thienyl, pyrrolyl, isoxazolyl, pyrazolyl, oxazolyl, isothiazolyl, thiazolyl, imidazolyl, triazolyl, oxadiazolyl, thiadiazolyl, teterazolyl, pyridyl, pyrimidinyl, pyridazinyl, pyrazinyl, triazinyl, indolyl, benzoxazolyl, benzothiazolyl, quinolyl and the like, and the heterocycle may be substituted.

If not defined otherwise, the term “which may be substituted”, which is the same as the term “optionally substituted”, indicates that the groups may be substituted by at least one substituent selected among the following substituents: Halogen, hydroxy, thiohydroxy, amino, cyano, nitro, C₁₋₁₂ alkyl (preferably C₁₋₆ alkyl, more preferably C₁₋₄ alkyl), C₁₋₁₂ haloalkyl (preferably C₁₋₆ haloalkyl, more preferably C₁₋₄ haloalkyl), C₁₋₁₂ alkoxy (preferably C₁₋₆ alkoxy; more preferably C₁₋₄ alkoxy), C₁₋₁₂ haloalkoxy (preferably C₁₋₆ haloalkoxy, more preferably C₁₋₄ haloalkoxy), C₁₋₁₂ alkylthio (preferably C₁₋₆ alkylthio, more preferably C₁₋₄ alkylthio), C₁₋₁₂ haloalkylthio (preferably C₁₋₆ haloalkylthio, more preferably C₁₋₄ haloalkylthio), C₁₋₁₂ alkylsulfinyl (preferably C₁₋₆ alkylsulfinyl, more preferably C₁₋₄ alkylsulfinyl), C₁₋₁₂ haloalkylsulfinyl (preferably C₁₋₆ haloalkylsulfinyl, more preferably C₁₋₄ haloalkylsulfinyl), C₁₋₁₂ alkylsulfonyl (preferably C₁₋₆ alkylsulfonyl, more preferably C₁₋₄ alkylsulfonyl) and C₁₋₁₂ haloalkylsulfonyl (preferably C₁₋₆ haloalkylsulfonyl, more preferably C₁₋₄ haloalkylsulfonyl).

For example, with respect to alkyl which “may be substituted with halogen”, (which results among other in haloalkyl groups), the following groups CH₂F, CHF₂, CF₃, CF₂Cl, CFCl₂, CF₂Br, CF₂CF₃, CFHCF₃, CH₂CF₃, CFClCF₃, CCl₂CF₃, CF₂CH₃, CF₂CH₂F, CF₂CHF₂, CF₂CF₂Cl, CF₂CF₂Br, CFHCH₃, CFHCHF₃, CFHCHF₂, CHFCF₃, CHFCF₂Cl, CHFCF₂Br, CFClCF₃, CCl₂CF₃, CF₂CF₂CF₃, CH₂CF₂CF₃, CF₂CH₂CF₃, CF₂CF₂CH₃, CHFCF₂CF₃, CF₂CHFCF₃, CF₂CF₂CHF₂, CF₂CF₂CH₂F, CF₂CF₂CF₂Cl, CF₂CF₂CF₂Br can be exemplified.

If not defined otherwise, “normal pressure” stands for “atmospheric pressure” i.e. around 1 bar.

If not explicitly mentioned otherwise, the term “agricultural” refers to crop production systems. Livestock productions systems are encompassed by the term “veterinary field”. Agricultural pests are therefore pests which occur in the crop production system.

The compounds according to the invention can be obtained according to known methods or by using the preparation methods described herein.

Thus, the invention is directed to a preparation method (a) for the preparation of compounds of formula (I) as defined herein, wherein l is 0, which preparation method (a) comprises reacting a compound of formula (II)

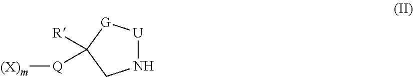

wherein X, m, Q, R′, G and U are as defined herein, with a compound of formula (III):

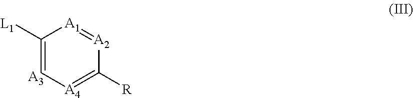

wherein the grouping

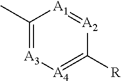

is as defined herein, and wherein L₁ represents halogen or C₁₋₄ haloalkylsulfonyloxy, in a diluent, and in the presence of at least one base and in the presence of a metal catalyst.

Moreover, the invention is directed to a preparation method (b) for the preparation of compounds of formula (I) as defined herein, wherein l is 0, and the grouping

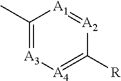

stands for a group [g1] as defined herein, which preparation method (b) comprises reacting a compound of formula (Ib)

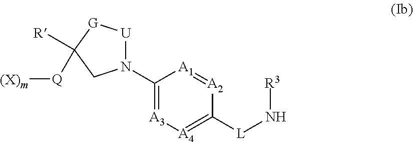

wherein X, m, Q, R′, G, U, L, R³, and A₁ to A₄ are as defined herein, with a compound of formula (VII)

R⁴-L₃ (VII)

wherein R⁴ is as defined herein and L₃ represents fluorine, chlorine, bromine, C₁₋₄ alkylcarbonyloxy, C₁₋₄ alkoxy-carbonyloxy, C₁₋₄ alkyl-sulfonyloxy, C₁₋₄ haloalkyl-sulfonyloxy, arylsulfonyloxy or azolyl, in a diluent, and optionally in the presence of at least one base.

The preparation method (a) is exemplified by the following scheme, wherein 5-(3,5-dichlorophenyl)-5-(trifluoromethyl)-1,3-oxazolidin-2-one and N-[4-bromo-2-(trifluoromethyl)benzyl]acetamide are used as starting materials.

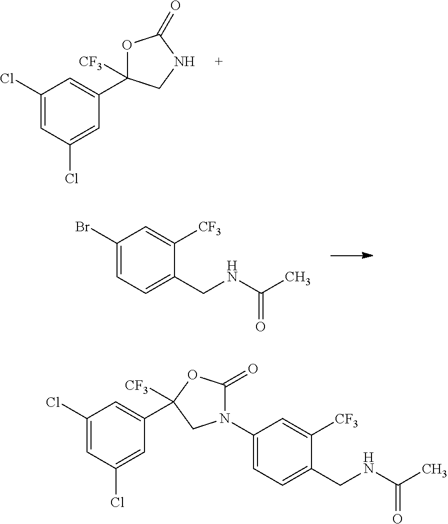

The preparation method (b) is exemplified by the following scheme, wherein 3-[4-(aminomethyl)-3-(trifluoromethyl)phenyl]-5-(3,5-dichlorophenyl)-5-(trifluoromethyl)-1,3-oxazolidin-2-one and acetyl chloride are used as starting materials.

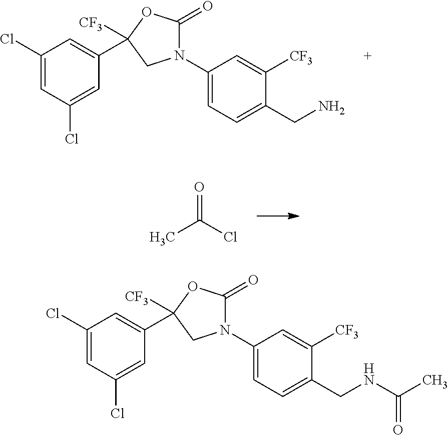

The preparation method (a) can be carried out according to the method described in WO 2007/123853A2 and WO 2008/128711, respectively. The compounds of the formula (II) in the preparation method (a) can be obtained by the methods described for example in: WO 2007/123853A2, the Journal of Organic Chemistry, 1991, Vol. 56, pages 7336-7340; Ibid. 1994, Vol. 59, pages 2898-2901; Journal of Fluorine Chemistry, 1999, Vol. 95, pages 167-170; WO 2005/05085216A; Chemistry Letters, 1984, 1117-1120; Tetrahedron Letters, 1993, 34, 3279-3282; Journal of the Organic Chemistry, 1984, 49, 2081; and Protective Groups in Organic Chemistry Third Edition, John Wiley & Sons, Inc.

A specific preparation method for compounds of the formula (IIb), which are encompassed by general formula (II) for the preparation method (a), is shown in the following reaction scheme 1:

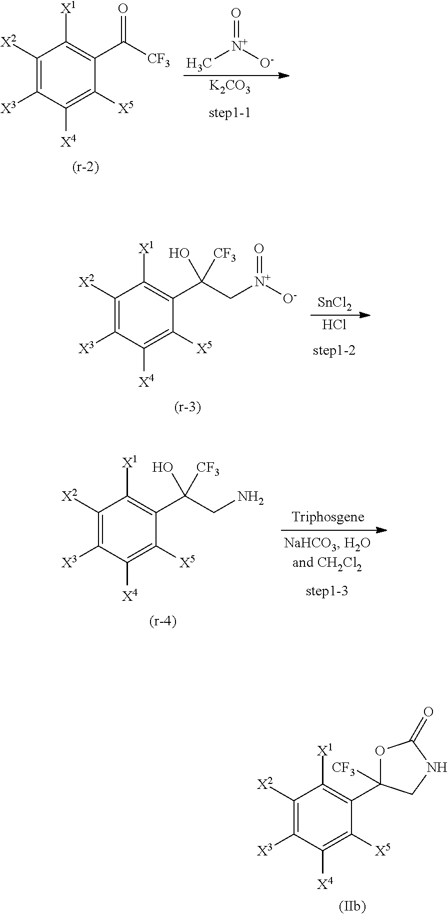

wherein, a compound of formula (r-2) and nitromethane are reacted in the presence of a base or bases, for example potassium carbonate, to obtain a compound of formula (r-3) (step 1-1). This compound of formula (r-3) is reduced by using an appropriate reducing agent, for example tin chloride, in the presence of hydrochloric acid to obtain a compound of formula (r-4) (step 1-2). The compound of formula (r-4) is finally reacted with triphosgene in the presence of a base or bases to obtain a compound of formula (IIb) (step 1-3).

Several compounds of formula (r-2) in reaction scheme 1 are known, and representative examples thereof include: 1-(3,5-dichlorophenyl)-2,2,2-trifluoroethanone, 2,2,2-trifluoro-1-(3,4,5-trichlorophenyl)ethanone, 2,2,2-trifluoro-1-[3-trifluoromethyl)phenyl]ethanone, 1-[3,5-bis(trifluoromethyl)phenyl]-2,2,2-trifluoro-ethanone and the like.

Some compounds of formula (r-3) in reaction scheme 1 are known, and representative examples thereof include: 1,1,1-trifluoro-3-nitro-2-(3,4,5-trichlorophenyl)propan-2-ol, 2-[3,5-bis(trifluoromethyl)phenyl]-1,1,1-trifluoro-3-nitropropan-2-ol and the like.

Some compounds of formula (r-4) in reaction scheme 1 are known, and representative examples thereof include: 3-amino-2-(3,5-dichlorophenyl)-1,1,1-trifluoropropan-2-ol, 3-amino-1,1,1-trifluoro-2-(3,4,5-trichlorophenyl)propan-2-ol, 3-amino-1,1,1-trifluoro-2-[3-(trifluoromethyl)phenyl]propan-2-ol, 3-amino-2-[3,5-bis(trifluoromethyl)phenyl]-1,1,1-trifluoropropan-2-ol and the like.

Some compounds of formula (IIb) in preparation method (a) and reaction scheme 1 are known, and representative examples thereof include: 5-(3,5-dichlorophenyl)-5-(trifluoromethyl)-1,3-oxazolidin-2-one, 5-(3,4,5-trichlorophenyl)-5-(trifluoromethyl)-1,3-oxazolidin-2-one, 5-(trifluoromethyl)-5-[3-(trifluoromethyl)phenyl]-1,3-oxazolidin-2-one, 5-[3,5-bis(trifluoromethyl)phenyl]-5-(trifluoromethyl)-1,3-oxazolidin-2-one and the like.

The compounds of formula (III) for preparation method (a) can be synthesized, for example, according to the methods described in WO 2007/105814A1. Additionally, they can be also easily synthesized according to known methods in the field of organic chemistry.

Some compounds of formula (III) are known, and representative examples thereof include

N-[4-bromo-2-(trifluoromethyl)benzyl]acetamide, N-(4-bromo-2-chlorobenzyl)acetamide, N-[4-bromo-2-(trifluoromethyl)benzyl]propane amide, N-(4-bromo-2-chlorobenzyl)propane amide,

N-[4-bromo-2-(trifluoromethyl)benzyl]butane amide, N-(4-bromo-2-chlorobenzyl)butane amide,

N-[4-bromo-2-(trifluoromethyl)benzyl]cyclopropanecarboxamide,

N-(4-bromo-2-chlorobenzyl)cyclopropanecarboxamide,

N-[4-bromo-2-(trifluoromethyl)benzyl]-2-cyclopropylacetamide,

N-(4-bromo-2-chlorobenzyl)-2-cyclopropylacetamide,

N-[4-bromo-2-(trifluoromethyl)benzyl]-3,3,3-trifluoropropane amide,

N-(4-bromo-2-chlorobenzyl)-3,3,3-trifluoropropane amide,

N-[4-bromo-2-(trifluoromethyl)benzyl]-2-(methylsulfanyl)acetamide,

N-(4-bromo-2-chlorobenzyl)-2-(methylsulfanyl)acetamide,

N-[4-bromo-2-(trifluoromethyl)benzyl]-2-(methylsulfinyl)acetamide,

N-(4-bromo-2-chlorobenzyl)-2-(methylsulfinyl)acetamide,

N-[4-bromo-2-(trifluoromethyl)benzyl]-2-(methylsulfonyl)acetamide,

N-(4-bromo-2-chlorobenzyl)-2-(methylsulfonyl)acetamide,

N-[4-bromo-2-(trifluoromethyl)benzyl]-3-methoxypropane amide,

N-(4-bromo-2-chlorobenzyl)-3-methoxypropane amide,

N-[4-bromo-2-(trifluoromethyl)benzyl]-3-methoxybutane amide,

N-(4-bromo-2-chlorobenzyl)-3-methoxybutane amide and the like.

The reaction of preparation method (a) is carried out in appropriate diluents such as aliphatic hydrocarbons (e.g. hexane, cyclohexane, heptane, etc.), aliphatic halogenated hydrocarbons (e.g. dichloromethane, chloroform, carbon tetrachloride, dichloroethane, etc.), aromatic hydrocarbons (e.g. benzene, toluene, xylene, chlorobenzene, etc.), ethers (e.g. diethyl ether, dibutyl ether, dimethoxyethane (DME), tetrahydrofuran, dioxane, etc.), esters (e.g. ethyl acetate, ethyl propionate, etc.), acid amides (e.g. dimethylformamide (DMF), dimethyl acetamide (DMA), N-methylpyrrolidone, etc.), nitriles (e.g. acetonitrile, propionitrile, etc.), dimethyl sulfoxide (DMSO), water, in mixtures of before mentioned diluents.

The reaction of the preparation method (a) can be carried out in the presence of a base or bases. Appropriate bases are, for example, alkali metal bases (e.g. lithium hydride, sodium hydride, potassium hydride, butyl lithium, tert-butyl lithium, trimethylsilyl lithium, lithium hexamethyldisilazide, sodium carbonate, potassium carbonate, cesium carbonate, tripotassium phosphate, sodium acetate, potassium acetate, sodium methoxide, sodium ethoxide, sodium-tert-butoxide and potassium-tert-butoxide); or organic bases (e.g. triethylamine, diisopropylethylamine, tributylamine, N-methylmorpholine, N,N-dimethylaniline, N,N-diethylaniline, 4-tert-butyl-N,N-dimethylaniline, pyridine, picoline, lutidine, diazabicycloundecene, diazabicyclooctane, imidazole, as well as 1,2-cyclohexanediamine, trans-1,2-cyclohexanediamine, and N,N-4-(dimethylamino)-pyridine (DMAP)).

The reaction can be carried out by using metal catalysts. The metal catalyst can be added as such or is produced in situ. Preferred are transition metal catalysts. Typical examples for such catalysts include Pd₂(dba)₃, Pd₂(dba)₃CHCl₃ (dba=dibenzylidene acetone), Pd(OAc)₂, CuI, and Cu₂O.

The reaction can also be carried out by using a phosphine ligand (e.g. (2,2′-bis(diphenylphosphino)-1,1′-binaphtalene (BINAP), or 4,5-bis(diphenylphosphino)-9,9-dimethylxanthene (Xantphos), or tributylphosphine), or an amine ligand (e.g. 8-quinolinol, proline, and N,N-dimethylglycine, trans-1,2-cyclohexanediamine), preferably in the presence of a transition metal catalyst (such as CuI). The ligands may also function as a base (cf. trans-1,2-cyclohexanediamine) in this reaction, so that no further base is needed.

The preparation method (a) can be carried out over substantially wide temperature range. It may be generally carried out at a temperature in the range of about −78° C. and about 200° C., preferably in the range of −10° C. and about 150° C. The reaction is preferably carried out at normal pressure although it may also be carried out under elevated or reduced pressure. The reaction time is from 0.1 to 72 hours, preferably 0.1 to 24 hours.

For carrying out the preparation method (a), for example, 1 mol of the compound of the formula (II) can be reacted with 1 to 3 mols of the compound of the formula (III), in the presence of 1 to 3 mols of a base (e.g. trans-1,2-cyclohexanediamine) and a metal catalyst (e.g. CuI) in a diluent (e.g. 1,4-dioxane) to obtain the compound according to the invention.

The compounds of the present invention can be obtained in the preparation method (a), by reacting 1 to 3 moles of the compound of the formula (III) in a diluent, for example toluene, with 1 mol of the compound of the formula VI), in the presence of 1 to 3 moles of bases and a catalytic amount of Pd₂(dba)₃CHCl₃, Xantphos.

The preparation method (b) can be carried out according to the method described in WO 2008/128711.

Compounds of the formula (Ib), which are the starting materials in the preparation method (b), are encompassed by formula (I).

A specific preparation method for compounds of formula (Ib-1), which are specific examples of the starting materials of formula (Ib), is shown in the following reaction scheme 2:

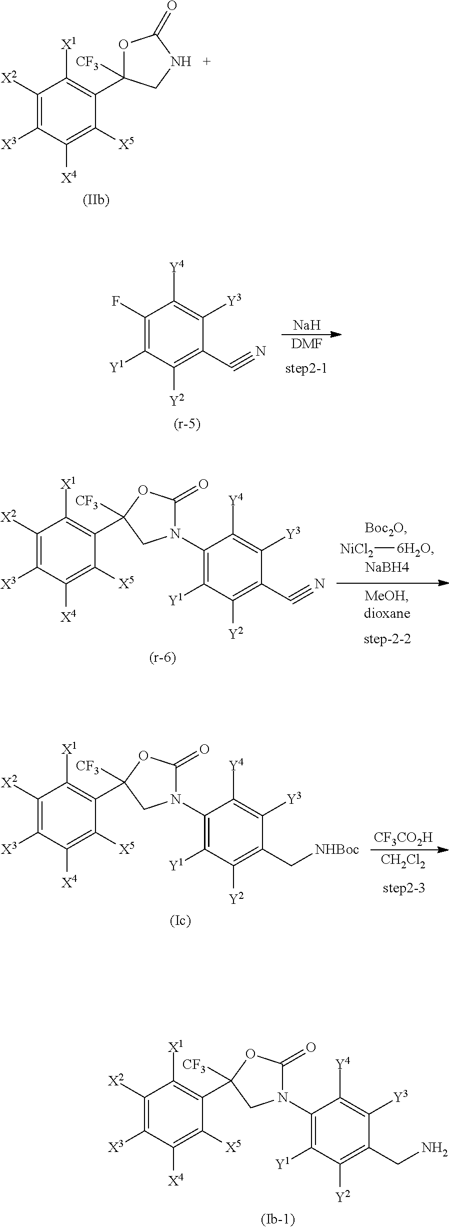

in reaction scheme 2 (step 2-1), a compound of the formula (IIb) and a compound of formula (r-5) are reacted in the presence of a base, for example sodium hydride, to give a compound of formula (r-6). According to step 2-2, the cyano group contained in the compound of the formula (r-6) is reduced by known methods (cf. Tetrahedron Letters, 2000, 41, 3513-3516 or Tetrahedron, 2003, 59, 5417-5423) resulting in a compound of formula (Ic). According to step 2-3, a compound of formula (Ic) is deprotected in the presence of a caralytic amount of an acid (acid catalysts), for example trifluoroacetic acid, to give a compound of formula (Ib-1). Compounds of the formula (Ic) are encompassed by formula (I).

Compounds of formula (r-5) are known. Representative examples include: 4-fluorobenzonitrile, 2-chloro-4-fluorobenzonitrile, 4-fluoro-2-(trifluoromethyl)benzonitrile.

Compounds of formula (r-6) are known. Representative examples include: 4-[5-(3,5-dichlorophenyl)-2-oxo-5-(trifluoromethyl)-1,3-oxazolinon-3-yl]-2-(trifluoromethyl)benzonitrile, 2-chloro-4-[2-oxo-5-(3,4,5-trichlorophenyl)-5-(trifluoromethyl)-1,3-oxazolidin-3-yl]benzonitrile, 4-[2-oxo-5-(3,4,5-trichlorophenyl)-5-(trifluoromethyl)-1,3-oxazolidin-3-yl]-2-(trifluoromethyl)benzonitrile, 2-chloro-4-{2-oxo-5-(trifluoromethyl)-5-[3-(trifluoromethyl)phenyl]-1,3-oxazolidin-3-yl}benzonitrile, 4-{2-oxo-5-(trifluoromethyl)-5-(trifluoromethyl)phenyl]-1,3-oxazolidin-3-yl}-2-(trifluoromethyl)benzonitrile, 4-{5-[3,5-bis(trifluoromethyl)phenyl]-2-oxo-5-(trifluoromethyl)1,3-oxazolidin-3-yl}-2-chlorobenzonitrile, 4-{5-[3,5-bis(trifluoromethyl)phenyl]-2-oxo-5(trifluoromethyl)-1,3-oxazolidin-3-yl}-2-(trifluoromethyl)benzonitrile.

Compounds of, formula (Ib) include: 3-[4-(aminomethyl)-3-(trifluoromethyl)phenyl]-5-(3,5-dichlorophenyl)-5-(trifluoromethyl)-1,3-oxazolidin-2-one, 3-[4-(aminomethyl)-3-(trifluoromethyl)phenyl]-5-(3,4,5-trichlorophenyl)-5-(trifluoromethyl)-1,3-oxazolidin-2-one, 3-[4-(aminomethyl)-3-chlorophenyl]-5-[3,5-bis(trifluoromethyl)phenyl]-5-(trifluoromethyl)-1,3-oxazolidin-2-one, 3-[4-(aminomethyl)-3-(trifluoroethyl)phenyl]-5-[3,5-bis(trifluoromethyl)phenyl]-5-(trifluoromethyl)-13-oxazolidin-2-one, 3-[4-(aminomethyl)-3-methylphenyl]-5-[3,5-bis(trifluoromethyl)phenyl]-5-(trifluoromethyl)-1,3-oxazolidin-2-one, 3-[4-(1-aminoethyl)phenyl]-5-(3,4,5-trichlorophenyl)-5-(trifluoromethyl)-1,3-oxazolidin-2-one.

Compounds of the formula (VII), which are the starting materials in the preparation method (b), are known. Examples thereof include acetyl chloride, and propanoyl chloride.

The same diluents as described for preparation method (a) can be used in preparation method (b).

The reaction of preparation method (b) is preferably carried out in the presence of appropriate bases. Appropriate bases include the bases which can be used in preparation (a).

The preparation method (b) can be carried out over substantially wide temperature range. It may be generally carried out between about −78° C. and about 200° C., preferably between about −10° C. and about 150° C. The reaction is preferably carried out at normal pressure although it may be also carried out under elevated or reduced pressure. The reaction time is 0.1 to 72 hours, preferably 0.1 to 24 hours.

For carrying out the preparation method (b), for example, 1 mol of a compound of the formula (Ib) is reacted with 1 to 5 mols of a compound of the formula (VII) in a diluent (e.g. THF) in the presence of a base.

A compound of formula (Id):

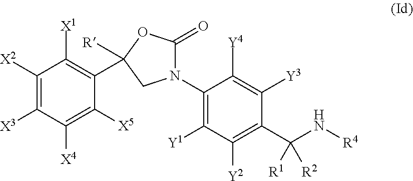

wherein X¹ to X⁵, R′, R¹, R², R⁴, and Y¹-Y⁴ are as defined herein, can be obtained through preparation method (b) by reacting 1 to 1.1 moles of a compound of formula (VII) in a diluent, for example THF, with 1 mol of a compound of formula (Ib) in the presence of a base. Compounds of the formula (Id) are encompassed by formula (I).

When R³ is other not hydrogen compounds of formula (I) can be prepared by reacting a compound of formula (Id), obtained according to preparation method (b) with a compound of formula (r-7):

R³-L₂ (r-7)

The compounds of the formula (r-7) are known, and examples thereof include methyl iodide, ethyl iodide and the like.

When N-oxides of the compounds of the formula (I) of the present invention are to be prepared, they can be obtained by oxidation of corresponding azolidine compounds based on general methods.

The active compounds according to the invention may be used in combination with suitable synergists or other active compounds, such as for example insecticides, acaricides, nematicides, fungicides, biological control agents, and bacterizides. Such combinations can also result in a synergistic effect, i.e. the biological activity of such a combination is synergistically increased. Examples of such combination partners are the following insecticides, acaricides, nematicides which are sorted by their mode of action:

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, and xylylcarb; or

organophosphates, e.g. acephate, azamethiphos, azinphos (-methyl, -ethyl), cadusafos, chlorethoxyfos, chlorfenvinphos, chlorfenvinphos, chlormephos, chlorpyrifos (-methyl), coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion (-methyl), phenthoate, phorate, phosalone, phosmet, phosphamidon, phoxim, pirimiphos (-methyl), profenofos, propetamphos, prothiofos, pyraclofos, pyridaphenthion, quinalphos, sulfotep, tebupirimfos, temephos, terbufos, tetrachlorvinphos, thiometon, triazophos, triclorfon, and vamidothion.

(2) GABA-gated chloride channel antagonists, for example organochlorines, e.g. chlordane, endosulfan (alpha-); or fiproles (phenylpyrazoles), e.g. ethiprole, fipronil, pyrafluprole, and pyriprole.

(3) Sodium channel modulators/voltage-dependent sodium channel blockers, for example pyrethroids, e.g. acrinathrin, allethrin (d-cis-trans, d-trans), bifenthrin, bioallethrin, bioallethrin S-cyclopentenyl, bioresmethrin, cycloprothrin, cyfluthrin (beta-), cyhalothrin (gamma-, lambda-), cypermethrin (alpha-, beta-, theta-, zeta-), cyphenothrin [(1R)-trans-isomers], deltamethrin, dimefluthrin, empenthrin [(EZ)-(1R)-isomers), esfenvalerate, etofenprox, fenpropathrin, fenvalerate, flucythrinate, flumethrin, fluvalinate (tau-), halfenprox, imiprothrin, metofluthrin, permethrin, phenothrin [(1R)-trans-isomer), prallethrin, profluthrin, pyrethrin (pyrethrum), resmethrin, RU 15525, silafluofen, tefluthrin, tetramethrin [(1R)-isomers)], tralomethrin, transfluthrin and ZXI 8901; or DDT; or methoxychlor.

(4) Nicotinergic acetylcholine receptor agonists, for example chloronicotinyls, e.g. acetamiprid, clothianidin, dinotcfuran, imidacloprid, nitenpyram, thiacloprid, thiamethoxam; or nicotine.

(5) Allosteric acetylcholine receptor modulators (agonists), for example spinosyns, e.g. spinetoram and spinosad.

(6) Chloride channel activators, for example avermectins/milbemycins, e.g. abamectin, emamectin benzoate, lepimectin, and milbemectin.

(7) Juvenile hormone mimics, e.g. hydroprene, kinoprene, methoprene; or fenoxycarb; pyriproxyfen.

(8) Miscellaneous non-specific (multi-site) inhibitors, for example gassing agents, e.g. methyl bromide and other alkyl halides; or chloropicrin; sulfuryl fluoride; borax; tartar emetic.

(9) Selective homopteran feeding blockers, e.g. pymetrozine or flonicamid.

(10) Mite growth inhibitors, e.g. clofentezine, diflovidazin, hexythiazox, etoxazole.

(11) Microbial disrupters of insect midgut membranes, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT crop proteins: Cry1Ab, Cry1Ac, Cry1Fa, Cry2Ab, mCry3A, Cry3Ab, Cry3Bb, Cry34/35Ab1.

(12) Inhibitors of mitochondrial ATP synthase, for example diafenthiuron; or organotin miticides, e.g. azocyclotin, cyhexatin, and fenbutatin oxide; or propargite; tetradifon.

(13) Uncouplers of oxidative phoshorylation via disruption of the proton gradient, for example chlorfenapyr, and DNOC.

(14) Nicotinic acetylcholine receptor channel blockers, for example bensultap, cartap hydrochloride, thiocyclam, and thiosultap-sodium.

(15) Inhibitors of chitin biosynthesis, type 0, for example benzoylureas, e.g. bistrifluron, chlorfluazuron, diflubenzuron, flucycloxuron, flufenoxuron, hexaflumuron, lufenuron, novaluron, noviflumuron, penfluron, teflubenzuron, and triflumuron.

(16) Inhibitors of chitin biosynthesis, type 1, for example buprofezin.

(17) Molting disrupters, for example cyromazine.

(18) Ecdysone receptor agonists/disrupters, for example diacylhydrazines, e.g. chromafenozide, halofenozide, methoxyfenozide, and tebufenozide.

(19) Octopamine receptor agonists, for example amitraz.

(20) Mitochondrial complex III electron transport inhibitors, for example hydramethylnon; acequinocyl or fluacrypyrim.

(21) Mitochondrial complex I electron transport inhibitors, for example METI acaricides, e.g. fenazaquin, fenpyroximate, pyrimidifen, pyridaben, tebufenpyrad, tolfenpyrad or rotenone. (Derris).

(22) Voltage-dependent sodium channel blockers, e.g. indoxacarb; metaflumizone.

(23) Inhibitors of acetyl CoA carboxylase, for example tetronic acid derivatives, e.g. spirodiclofen and spiromesifen; or tetramic acid derivatives, e.g. spirotetramat.

(24) Mitochondrial complex IV electron inhibitors, for example phosphines, e.g. aluminium phosphide, calcium phosphide, phosphine, and zinc phosphide or cyanide.

(25) Mitochondrial complex II electron transport inhibitors, for example cyenopyrafen.

(28) Ryanodine receptor modulators, for example diamides, e.g. chlorantraniliprole (Rynaxypyr), Cyantraniliprole (Cyazypyr), and flubendiamide.

Further active ingredients with unknown or uncertain mode of action, for example azadirachtin, amidoflumet, benzoximate, bifenazate, chinomethionat, cryolite, cyflumetofen, dicofol, fluensulfone (5-chloro-2-[(3,4,4-trifluorobut-3-en-1-yl)sulfonyl]-1,3-thiazole), flufenerim, pyridalyl, and pyrifluquinazon; furthermore products based on Bacillus firmus (I-1582, BioNeem, Votivo) or one of the following known active compounds:

4-{[(6-brompyrid-3-yl)methyl](2-fluorethyl)amino}furan-2(5H)-on (known from WO 2007/115644), 4-{[(6-fluorpyrid-3-yl)methyl](2,2-difluorethyl)amino}furan-2(5H)-on (known from WO 2007/115644), 4-{[(2-chlor-1,3-thiazol-5-yl)methyl](2-fluorethyl)amino}furan-2(5H)-on (known from WO 2007/115644), 4-{[(6-chlorpyrid-3-yl)methyl](2-fluorethyl)amino}furan-2(5H)-on (known from WO 2007/115644), 4-{[(6-chlorpyrid-3-yl)methyl](2,2-difluorethyl)amino}furan-2(5H)-on known from WO 2007/115644), 4-{[(6-chlor-5-fluorpyrid-3-yl)methyl](methyl)amino}furan-2(5H)-on (known from WO 2007/115643), 4-{[(5,6-dichlorpyrid-3-yl)methyl](2-fluorethyl)amino}furan-2(5H)-on (known from WO 2007/115646), 4-{[(6-chlor-5-fluorpyrid-3-yl)methyl](cyclopropyl)amino}furan-2(5H)-on (known from WO 2007/115643), 4-{[(6-chlorpyrid-3-yl)methyl](cyclopropyl)amino}furan-2(5H)-on (known from EP-A-0 539 588), 4-{[(6-chlorpyrid-3-yl)methyl](methyl)amino}furan-2(5H)-on (known from EP-A-0 539 588), [(6-chlorpyridin-3-yl)methyl](methyl)oxido-λ4-sulfanylidencyanamid (known from WO 2007/149134), [1-(6-chlorpyridin-3-yl)ethyl](methyl)oxido-λ4-sulfanylidencyanamid (known from WO 2007/149134) and its diastereomeres (A) and (B)

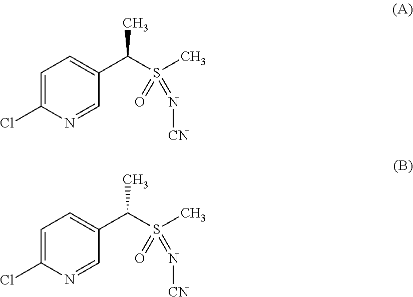

(also known from WO 2007/149134), [(6-trifluormethylpyridin-3-yl)methyl](methyl)oxido-λ4-sulfanylidencyanamid (known from WO 2007/095229), or sulfoxaflor (also known from WO 2007/149134), 11-(4-chloro-2,6-dimethylphenyl)-12-hydroxy-1,4-dioxa-9-azadispiro[4.2.4.2]tetradec-11-en-10-one (known from WO 2006/089633), 3-(4′-fluoro-2,4-dimethylbiphenyl-3-yl)-4-hydroxy-8-oxa-1-azaspiro[4.5]dec-3-en-2-one (known from WO 2008/067911), and 1-[2-fluoro-4-methyl-5-[(2,2,2-trifluoroethyl)sulfinyl]phenyl]-3-(trifluoromethyl)-1H-1,2,4-Triazol-5-amine (known from WO 2006/043635). Examples of further combination partners are the following fungicides:

(1) Inhibitors of the ergosterol biosynthesis, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-[1-(4-methoxyphenoxy)-3,3-dimethylbutan-2-yl]1H-imidazole-1-carbothioate.

(2) inhibitors of the respiratory chain at complex I or II, for example bixafen, boscalid, carboxin, diflumetorim, fenfuram, fluopyram, flutolanil, fluxapyroxad, furametpyr, furmecyclox, isopyrazam (mixture of syn-epimeric racemate 1RS,4SR,9RS and anti-epimeric racemate 1RS,4SR,9SR), isopyrazam (anti-epimeric racemate 1RS,4SR,9SR), isopyrazam (anti-epimeric enantiomer 1R,4S,9S), isopyrazam (anti-epimeric enantiomer 1S,4R,9R), isopyrazam (syn epimeric racemate 1RS,4SR,9RS), isopyrazam (syn-epimeric enantiomer 1R,4S,9R), isopyrazam (syn-epimeric enantiomer 1S,4R,9S), mepronil, oxycarboxin, penflufen, penthiopyrad, sedaxane, thifluzamide, 1-methyl-N-[2-(1,1,2,2-tetrafluoroethoxy)phenyl]-3-(trifluoromethyl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(di fluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide and salts thereof.

(3) inhibitors of the respiratory chain at complex III, for example ametoctradin, amisulbrom, azoxystrobin, cyazofamid, dimoxystrobin, enestroburin, famoxadone, fenamidone, fluoxastrobin, kresoxim-methyl, metominostrobin, orysastrobin, picoxystrobin, pyraclostrobin, pyrametostrobin, pyraoxystrobin, pyribencarb, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}-imino)methyl]phenyl}ethanamide, (2E)-2-{2-[({[(1E)-1-(3-{[(E)-1-fluoro-2-phenylethenyl]oxy}-phenyl)ethylidene]amino}oxy)methyl]phenyl}-2-(methoxyimino)-N-methylethanamide, (2E)-2-{2-[-({[(2E,3 E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, methyl(2E)-2-{2-[({cyclopropyl[(4-methoxyphenyl)imino]methyl}-sulfanyl)methyl]phenyl}-3-methoxyprop-2-enoate, N-(3-ethyl-3,5,5-trimethylcyclohexyl)-3-(formylamino)-2-hydroxybenzamide, 2-{2-[(2,5-dimethylphenoxy)methyl]phenyl}-2-methoxy-N-methylacetamide, (2R)-2-{2-[(2,5-dimethylphenoxy)methyl]phenyl}-2-methoxy-N-methylacetamide and salts thereof.

(4) Inhibitors of the mitosis and cell division, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)-[1,2,4]triazolo[1,5-a]pyrimidine, 3-chloro-5-(6-chloropyridin-3-yl)-6-methyl-4-(2,4,6-trifluorophenyl)-pyridazine and salts thereof.

(5) Compounds capable to have a multisite action, like for example bordeaux mixture, captafol, captan, chlorothalonil, copper hydroxide, copper naphthenate, copper oxide, copper oxychloride, copper(2+) sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulphur and sulphur preparations including calcium polysulphide, thiram, tolylfluanid, zineb, ziram and salts thereof.

(6) Compounds capable to induce a host defense, like for example acibenzolar-S-methyl, isotianil, probenazole, tiadinil and salts thereof.

(7) Inhibitors of the amino acid and/or protein biosynthesis, for example andoprim, blasticidin-S, cyprodinil, kasugamycin, kasugamycin hydrochloride hydrate, mepanipyrim, pyrimethanil and salts thereof.

(8) Inhibitors of the ATP production, for example fentin acetate, fentin chloride, fentin hydroxide and silthiofam.

(9) Inhibitors of the cell wall synthesis, for example benthiavalicarb, dimethomorph, flumorph, iprovalicarb, mandipropamid, polyoxins, polyoxorim, validamycin A and valifenalate.

(10) Inhibitors of the lipid and membrane synthesis, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.

(11) Inhibitors of the melanine biosynthesis, for example carpropamid, diclocymet, fenoxanil, phthalide, pyroquilon and tricyclazole.

(12) Inhibitors of the nucleic acid synthesis, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.

(13) Inhibitors of the signal transduction, for example chlozolinate, fenpiclonil, fludioxonil, iprodione, procymidone, quinoxyfen and vinclozolin.

(14) Compounds capable to act as an uncoupler, like for example binapacryl, dinocap, ferimzone, fluazinam and meptyldinocap.

(15) Further compounds, like for example benthiazole, bethoxazin, capsimycin, carvone, chinomethionat, chlazafenone, cufraneb, cyflufenamid, cymoxanil, cyprosulfamide, dazomet, debacarb, dichlorophen, diclomezine, difenzoquat, difenzoquat methylsulphate, diphenylamine, ecomate, fenpyrazamine, flumetover, fluoroimide, flusulfamide, flutianil, fosetyl-aluminium, fosetyl-calcium, fosetyl-sodium, hexachlorobenzene, irumamycin, methasulfocarb, methyl isothiocyanate, metrafenone, mildiomycin, natamycin, nickel dimethyldithiocarbamate, nitrothal-isopropyl, oethilinone, oxamocarb, oxyfenthiin, pentachlorophenol and salts, phenothrin, phosphorous acid and its salts, propamocarb-fosetylate, propanosine-sodium, proquinazid, pyrrolnitrine, tebufloquin, tecloftalam, tolnifanide, triazoxide, trichlamide, zarilamid, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-(4-{4-[(5 S)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]pyridine, 2-phenylphenol and salts, 3,4,5-trichloropyridine-2,6-dicarbonitrile, 3-[5-(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-methyl-6-octyl[1,2,4]triazolo[1,5-a]pyrimidin-7-amine, ethyl(2Z)-3-amino-2-cyano-3-phenylprop-2-enoate, N-(4-chlorobenzyl)-3-[3-methoxy-4-(prop-2-yn-1-yloxy)phenyl]propanamide, N-[(4-chlorophenyl)(cyano)-methyl]-3-[3-methoxy-4-(prop-2-yn-1-yloxy)phenyl]propanamide, N-[(5-bromo-3-chloropyridin-2-yl)-methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide.

N-{(E)-[(cyclopropylmethoxy)imino][6-(difluoromethoxy)-2,3-difluorophenyl]methyl}-2-phenylacetamide, N-{(Z)-[(cyclopropylmethoxy)imino][6-(difluoromethoxy)-2,3-difluorophenyl]methyl}-2-phenylacetamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, pentyl{6-[([{[(1-methyl-1H-tetrazol-5-yl)(phenyl)methylidene]amino}oxy)methyl]pyridin-2-yl}carbamate, phenazine-1-carboxylic acid, quinolin-8-ol and quinolin-8-ol sulfate (2:1).

(16) Further compounds like for example

1-methyl-3-(trifluoromethyl)-N-[2′-(trifluoromethyl)biphenyl-2-yl]-1H-pyrazole-4-carboxamide, N-(4′-chlorobiphenyl-2-yl)-3-(difluoromethyl)-1-methyl-1H-pyrazole-4-carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1-yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N -(4′-ethynylbiphenyl-2-yl)-5-fluoro-1,3-dimethyl-1H-pyrazole-4-carboxamide, 2-chloro-N-(4′-ethynylbiphenyl-2-yl)pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1 H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1 H-pyrazole-4-carboxamide,

5-fluoro-N-[4′-(3-methoxy-3-Methylbut-1-yn-1-yl)biphenyl-2-yl]-1,3-dimethyl-1H-pyrazole-4-carboxamide, 2-chloro-N-[4′-(3-methoxy-3-methylbut-1-yn-1-yl)biphenyl-2-yl]pyridine-3-carboxamide, (5-bromo-2-methoxy-4-methylpyridin-3-yl)(2,3,4-trimethoxy-6-methylphenyl)methanone and N-[2-(4-{[3-(4-chlorophenyl)prop-2-yn-1-yl]oxy}-3-methoxyphenyl)ethyl]-N2-(methylsulfonyl)valinamide.

The active ingredients specified herein by their “common name” are known and described, for example, in the Pesticide Manual (“The Pesticide Manual”, 14th Ed., British Crop Protection Council 2006) or can be searched in the internet (e.g. http://www.alanwood.net/pesticides).

The compounds of the present invention have a potent pesticidal effect. Therefore, the compounds of the present invention can be used as pesticides. Further, the compounds of the present invention exert a potent controlling effect against harmful agricultural pests without exhibiting a side effect of the compounds on cultivated crop plants and beneficials (e.g bees and humple bees). Therefore, the compounds of the present invention can be used for pest control against a wide variety of harmful organisms such as harmful sucking insects, chewing insects and other plant parasitic pests, stored grain pests, hygienic pests etc., and can be used for the disinfection and destruction of them. Such harmful organisms may be illustrated by the examples as follows:

As an insect, Coleoptera, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; Lepidoptera, for example, Lymantria dispar, Malacosoma neustria, Pieris rapae, Spodoptera litura, Mamestra brassicae, Chilo suppressalis, Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella; Hemiptera, for example, Nephotettix cincticeps, Nilaparvata lugens, Pseudococcus comstocki, Unapsis yanonensis, Myzus persicas, Aphis pomi, Aphis gossypii, Rhopalosiphum pseudobrassicas, Stephanitis nashi, Nezara spp., Trialeurodes vaporariorm, Psylla spp.; Thysanoptera, for example, Thrips palmi, Franklinella occidental; Orthoptera, for example, Blatella germanica, Periplaneta americana, Gryllotalpa Africana, Locusta migratoria migratoriodes; Isoptera, for example, Reticulitermes speratus, Coptotermes formosanus; Diptera, for example, Musca domestica, Aedes aegypti, Hylemia platura, Culex pipiens, Anopheles sinensis, Culex tritaeniorhynchus, Liriomyza torifolii.

As Acarina, Tetranychus cinnabarinus, Tetranychus urticae, Panonychus citri, Aculops pelekassi, Tarsonemus spp. can be mentioned.

As nematodes, Meloidogyne incognita, Bursaphelenchus lignicolus Mamiya et Kiyohara, Aphelenchoides besseyi, Heterodera glycines, Pratylenchus spp. can be mentioned.

The compounds of the present invention have good tolerance in plants and low toxicity to warm-blooded animals. Further, as being well received by an environment, the compounds of the present invention are appropriate for the protection of plants and plant parts.

With application of the compounds of the present invention, both crop Yield and quality of harvested products may be improved. In addition, the compounds of the present invention are suitable for protection of preserved products and materials and for a hygiene field, in terms of controlling harmful animals, in particular insects, spider-like animals, helminth, nematodes and mollusks that are encountered in agriculture, horticulture, veterimary medicine, forrest, garden and entertainment facilities. The compounds of the present invention can be preferably used as agents for protecting plants. The compounds of the present invention have an activity for normal sensitive species or resistant species, and for all over or several growth stages thereof. In particular, the harmful organisms mentioned above include the followings.

As Anoplura (Phthiraptera), for example, Damalinia spp., Haematopinus; Linognathus spp., Pediculus spp., Trichodectes spp.

As Arachnid, for example, Acarus siro, Aceria sheldoni. Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranyctus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

As Bivalvia, for example, Dreissena spp.

As Chilopoda, for example, Geophilus spp., Scutigera spp.

As Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnostema consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

As Collembola, for example, Onychiurus armatus.

As Dermaptera, for example, Forficula auricularia.

As Diplopoda, for example, Blaniulus guttulatus.

As Diptera, for example, Aedes spp., Anopheles spp., Bibio hortul anus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp, Oscinella frit, Pegomyia hyoscyami, Phorbia spp., Stomoxys spp., Tabanus spp., Tannia spp., Tipula paludosa, Wohlfahrtia spp.

As Gastropoda, for example, Arion spp., Biomphalaria spp., Bulinus spp., Deroceras spp., Galba spp., Lymnaea spp., Oncomelania spp., Succinea spp.

As Helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medeinensis, Echinococcus granulosus, Echinococcus multiocularis, Enterobius vermicularis, Faciola spp., Haemonchus spp., Heterakis spp., Hymenolepis nana, Hyostrongulus spp., Loa loa, Nematodirus spp., Oesophagostomum spp., Opisthorchis spp., Onchocerca volvulus, Ostertagia spp., Paragonimus spp., Schistosomen spp., Strongyloides fuellebomi, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

In addition, protozoa like Eimeria, etc. can be also controlled.

As Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus. spp., Euschistus spp., Eurygaster spp., Heliopeltis spp., Horchias nobiellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodonius spp., Sahlbergella singularis, Scotino phora spp., Stephanitis nashi, Tibraca spp., Triatoma spp.

As Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma pin, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Cameocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccu spp., Chryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratorioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesda gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolli.

As Hymenoptera, for example, Diprion spp., Hoplocampa spp., Lasius spp., Monomorium pharaonis and Vespa spp.

As Isopoda, for example, Armadillidium vulgare, Oniscus asellus, Porcellio scaber.

As Isoptera, for example, Reticulitermes spp., Odontotermes spp.

As Lepidoptera, for example, Acronicta major, Acdia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias in sulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

As Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

As Siphonaptera, for example, Ceratophyllus spp., Xenopsylla cheopis.

As Symphyla, for example, Scutigerella immaculata.

As Thysanoptera, for example, Baliothrips biformis, Enneothrips Havens, Frankliniella spp., Heliothrips spp., Hercinothrips femoralis, Kakothrips spp., Rhipiphorothrips cruentatus, Scirtothrips spp., Taeniothrips cardamoni, Thrips spp.

As Thysanura, for example, Lepisma saccharina.

As plant parasitic nematodes, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp. are included.

Additionally, the compounds according to the present invention show a potent insecticidal action and can, therefore, be used as an insecticide. Furthermore, the compounds according to the present invention exhibit a strong control effect against harmful animal pests, in particular arthropods and/or insects, particularly to agricultural pests, without imposing any harmful side effects of drug to the animal or the cultivated plants. The compounds of the present invention can thus be used for the control of a wide range of pest species, for example, harmful sucking insects, chewing insects, as well as other plant parasitic pests, storage insects, hygiene pests and the like, and can he applied for the purpose of disinfestations and extermination thereof.

The active compounds and active compound combinations according to the invention, in combination with good plant tolerance and favourable toxicity to warm-blooded animals and being tolerated well by the environment, are suitable for protecting plants and plant organs, for increasing harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They can be preferably employed as plant protection agents. They are active against normally sensitive and resistant species and against all or some stages of development. The above mentioned pests include:

Order: Arthropoda: From the class of the Arachnida, for example Acarus spp., Aceria sheldoni, Aculops spp., Aculus spp., Amblyomma spp., Amphitetranychus viennensis, Argas spp., Boophilus spp., Brevi-palpus spp., Bryobia praetiosa, Centruroides spp., Chorioptes spp., Dermanyssus gallinae, Dermatophagoides pteronyssius, Dermatophagoides farinae, Dermacentor spp., Eotetranychus spp., Epitrimerus pyri, Eutetranychus spp., Eriophyes spp., Halotydeus destructor, Hemitarsonemus spp., Hyalomma spp., Ixodes spp., Latrodectus spp., Loxosceles spp., Metatetranychus spp., Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vaejovis spp., Vasates lycopersici.

From the order of the Anoplura (Phthiraptera), for example, Damalinia spp., Haematopinus spp., Linognathus spp., Pediculus spp., Ptirus pubis, Trichodectes spp.

From the order of the Chilopoda, for example, Geophilus spp., Scutigera spp.

From the order of the Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptorhynchus lapathi, Cylindrocopturus spp., Dermestes spp., Diabrotica spp., Dichocrocis spp., Diloboderus spp., Epilachna spp., Epitrix spp., Faustinus spp., Gibbium psylloides, Hellula undalis, Heteronychus arator, Heteronyx spp., Hylamorpha elegans, Hylotrupes bajulus, Hypera postica, Hypothenemus spp., Lachnosterna consanguinea, Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Mclolontha spp., Migdolus spp., Monochamus spp., Naupactus xanthographus, Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllotreta spp., Popillia japonica, Premnotrypes spp., Prostephanus truncatus, Psylliodes spp., Ptinus spp., Rhizobius ventralis, Rhizopertha dominica, Sitophilus spp., Sphenophorus spp., Stegobium paniceum, Sternechus spp., Symphyletes spp., Tanymecus spp., Tenebrio molitor, Tribolium spp., Trogoderma spp., Tychius spp., Xylotrechus spp., Zabrus spp.

From the order of the Collembola, for example, Onychiurus armatus.

From the order of the Diplopoda, for example, Blaniulus guttulatus.

From the order of the Diptera, for example, Aedcs spp., Agromyza spp., Anastrepha spp., Anopheles spp., Asphondylia spp., Bactrocera spp., Bibio hortulanus, Calliphora erythrocephala, Ceratitis capitata, Chironomus spp., Chrysomyia spp., Chrysops spp., Cochliomyia spp., Contarinia spp., Cordylobia anthropophaga, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hylemyia spp., Hyppobosca spp., Hypoderma spp., Liriomyza spp. Lucilia spp., Lutzomia spp., Mansonia spp., Musca spp., Nezara spp., Oestrus spp., Oscinella frit, Pegomyia spp., Phlebotomus spp., Phorbia spp., Phormia spp., Prodiplosis spp., Psila rosae, Rhagoletis spp., Sarcophaga spp., Simulium spp, Stomoxys spp., Tabanus spp., Tannia spp., Tetanops spp., Tipula spp.

From the order of the Heteroptera, for example, Anasa tristis, Antestiopsis spp., Boisea spp., Blissus spp., Calocoris spp., Campylomma livida, Cavelerius spp., Cimex spp., Cimex lectularius, Cimex hemipterus, Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.

From the order of the Homoptera, for example, Acyrthosipon spp., Acrogonia spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Ferrisia spp., Geococcus coffeae, Hieroglyphus spp., Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes spp., Trioza spp., Typhlocyba spp., Unaspis spp., Viteus vitifolii, Zygina spp.

From the order of the Hymenoptera, for example, Acromyrmex spp., Athalia spp., Atta spp., Diprion spp., Hoplocampa spp., Lasius spp., Monomorium pharaonis, Solenopsis invicta, Tapinoma spp., Vespa spp.

From the order of the Isopoda, for example, Armadillidium vulgare, Oniscus asellus, Porcellio scaber.

From the order of the Isoptera, for example, Coptotermes spp., Cornitermes cumulans, Cryptotermes spp., Incisitermes spp., Microtermes obesi, Odontotermes spp., Reticulitermes spp.

From the order of the Lepidoptera, for example, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamestra brassicae, Mocis spp., Mythimna separata, Nymphula spp., Oiketicus spp., Oria spp., Orthaga spp., Ostrinia spp., Oulema oryzae, Panolis flammea, Pamara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudoplusia includens, Pyrausta nubilalis, Rachiplusia nu, Schoenobius spp., Scirpophaga spp., Scotia segetum, Sesamia spp., Sparganothis spp., Spodoptera spp., Stathmopoda spp., Stomopteryx subsecivella, Synanthedon spp., Tecia solanivora, Thermesia gemmatalis, Tinea pellionella, Tineola bisselliella, Tortrix spp., Trichophaga tapetzella, Trichoplusia spp., Tuta absoluta, Virachola spp.

From the order of the Orthoptera, for example, Acheta domesticus, Blatta orientalis, Blattella germanica, Dichroplus spp., Gryllotalpa spp., Leucophaea maderae, Locusta spp., Melanoplus spp., Periplaneta spp., Pulex irritans, Schistocerca gregaria, Supella longipalpa.

From the order of the Siphonaptera, for example, Ceratophyllus spp., Ctenocephalides spp., Tunga penetrans, Xenopsylla cheopis.

From the order of the Symphyla, for example, Scutigerella spp.

From the order of the Thysanoptera, for example, Anaphothrips obscurus, Baliothrips biformis, Drepanothris reuteri, Enneothrips Havens, Frankliniella spp., Heliothrips spp., Hercinothrips femoralis, Rhipiphorothrips cruentatus, Scirtothrips spp., Taeniothrips cardamoni, Thrips spp.

From the order of the Zygentoma (=Thysanura), for example, Lepisma saccharina, Thermobia domestica.

Order: Mollusca: From the class of the Bivalvia, for example, Dreissena spp.

From the class of the Gastropoda, for example, Anion spp., Biomphalaria spp., Bulinus spp., Deroceras spp., Galba spp., Lymnaea spp., Oncomelania spp., Pomacea spp., Succinea spp.

Order: Plathelminthes, Nematodes (animal parasites)

From the class of the Helminths, for example, Ancylostoma duodenale, Ancylostoma ceylanicum, Acylostoma braziliensis, Ancylostoma spp., Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

Order: Nematodes (plant parasites, phytoparasites)

From the group of the phytoparasitic nematodes, for example, Aphelenchoides spp., Bursaphelenchus spp., Ditylenchus spp., Globodera spp., Heterodera spp., Longidorus spp., Meloidogyne spp., Pratylenchus spp., Radopholus similis, Trichodorus spp., Tylenchulus semipenetrans, Xiphinema spp.

Subphylum: Protozoa: It is furthermore possible to control protozoa, such as Eimeria.

Any kind of plant and plant part can be treated according to the present invention. In the present invention, a plant should be understood as all plants and plant populations including desirable and undesirable wild plants or crop plants (including naturally-occurring crop plants) and the like. As for the crop plants, they can be plants which are obtainable by conventional methods of breeding modified varieties and optimization methods, or biotechnological methods and genetic engineering methods, or by combination of these methods, and they include transgenic plants. In addition, plant varieties which are either protected or not protected by a plant breeder are also included. Plant parts should be understood as all parts and organs of a plant that are present above or under ground. Examples thereof include shoots, leaves, flowers and roots, etc. Specific examples thereof include a leaf, a needle-like leaf, a stem, a trunk, a flower, a fruit, a fruit body, a seed, a root, a tuber and a underground tuber, etc. Plant parts also include a harvested material and a material which propagates sexually or asexually, for example, a cutting, a tuber, an underground tuber, a side branch and a seed.

Treatment of plants and plant parts with the active compounds according to the present invention can be carried out directly or by using conventional methods such as impregnation, spray, evaporation, particularization, dispersion, coating and injection, or for a propagating material, especially for a seed, by coating it with one or more of the compounds, so that the compounds are applied to their surroundings, habitat environment, or preservation place.

The compounds of the present invention have a penetrating activity and this means that the compounds can penetrate a plant body and can migrate from the underground part to the above-ground part of a plant.

As it has been described herein, according to the present invention, all plants and parts thereof can be treated. According to a preferred embodiment for carrying out the invention, wild plant species and plant mutants, or those obtained by traditional plant breeding methods such as hybridization or protoplast fusion, and parts thereof are treated. According to a more preferred embodiment for carrying out the invention, transgenic plants and plant varieties (genetically modified organisms) obtained by conventional methods in appropriate combination with genetic engineering methods, and parts thereof are treated. The terms “parts”, “parts of a plant” and “plant parts” are as defined above.

Still more preferably, for each specific case, plants of plant varieties that are commercially available or currently in use are treated according to the present invention. Plant varieties are understood as plants having new characteristics (“traits”) obtained by conventional breed improvements, introduction of mutation or recombinant DNA techniques. They can be plant varieties, biotypes or genotypes.

Depending on plant species or plant varieties, their habitat and growth condition (soil, weather, growth period, nutrition, etc.), the treatment according to the present invention may have a supra-additive (“synergy”) effect. Thus, for example, exceeding an expected effect, it is possible to obtain several effects including reduction of application rate and/or broadening of an activity spectrum, and/or increased activity of the material and composition that can be used according to the present invention, improvement of plant growth, enhancement of tolerance to high or low temperature, enhancement of tolerance to drought, moisture or salt contained in soil, improvement of a flowering property, simplification of harvest methods, accelerated maturation, increased harvest amount, improvement of quality and/or nutritional value of harvest products, and improvement of preservation stability and/or proccessability of harvested products.

The preferable transgenic plants or plant varieties (obtainable by genetic engineering methods) treated according to the present invention include all kinds of plant having genetic materials that can provide the plants with very advantageous and useful traits based on genetic modifications. Examples of such traits include improvement of plant growth, enhancement of tolerance to high or low temperature, enhancement of tolerance to drought, moisture or salt contained in soil, improvement of a flowering property, simplification of harvest methods, accelerated maturation, increased harvest amount, improvement of quality and/or nutritional value of harvest products, and improvement of preservation stability and/or proccessability of harvested products. Further examples in which such traits are particularly more emphasized include improved protection of plants against harmful animals and harmful microorganisms such as insect, tick, plant pathogenic fungus, bacteria and/or virus, and improved tolerance of plants against compounds having certain type of herbicidal activities. Examples of the transgenic plant include grain crops (barley, rice), corn, soybean, potato, sugar beet, tomato, bean and other modified plant species, useful plants such as cotton, tobacco, rape, and fruit plants (fruits like an apple, a pear, a citrus fruit and other fruit-bearing plants like a grape). In particular, corn, soybean, potato, cotton, tobacco and rape are important. As for the traits considered to be important, improved plant defense based on toxins produced by plants, in particular based on the toxins produced by plants with an action of genetic materials derived from Bacillus thuringiensis (for example, genes including CryIA(a), CryIA(b), CryIA(c), CryIIA, CryIIIA, CryIIIB2, Cry9c, Cry2Ab, Cry3Bb and CryIF, and combination thereof), against insects, spider-like animals, nematodes, slugs, and snails (herein herein, referred to as “Bt plant”) can be mentioned. Other traits considered to be important include improved plant defense against fungus, bacteria and virus, based on systemic acquired resistance (SAR), systemin, phytoallexin, elicitor, resistance gene and the corresponding protein and toxin expressed from the gene. Further, particularly important traits are improved tolerance of plants to a certain kind of an active compound having a herbicidal activity, such as imidazolinone, sulfonyl urea, glyphosate or phosphinotricine (e.g., “PTA” gene). Genes which can endow desired traits to a subject can also be present in combination each other in a transgenic plant. Examples of the “Bt plant” include modified varieties of corn, modified varieties of cotton and modified varieties of potato that are commercially available under the trade names of YIELD GARD® (for example, corn, cotton, soybean), KnockOut® (for example, corn), StarLink® (for example, corn), Bollgard® (cotton), Nucotn® (cotton) and New Leaf® (potato), respectively. Examples of the plant having resistance to herbicides include modified varieties of corn, modified varieties of cotton and modified varieties of potato that are commercially available under the trade names of Roundup Ready® (resistance to glyphosate, for example, corn, cotton, soybean), Liberty Link® (resistance to phosphinotricine, for example rape), IMI® (resistance to imidazolinones) and STS® (resistance to sulfonyl urea, for example, corn), respectively. Examples of the plant having resistance to herbicides (i.e., the plant obtained by conventional breeding methods to have resistance to herbicides) also include modified varieties, for example those that are commercially available under the trade name of Clearfield® (for example, corn). Of course, these descriptions are also applied to plant varieties which have already had genetic traits or will have genetic traits to be developed in future. Such plant varieties will be developed and/or on the market in future.

With the compounds of the present invention at appropriate concentration, the plants mentioned above can be advantageously treated, in particular.

Further, in the field of veterinary medicine, the novel compounds of the present invention can be effectively used against various harmful animal parasites (endo- and ectoparasites), for example, insects and helminths. Examples of such harmful animal parasites include the harmful organisms as follows. As insects, there are for example, Gasterophilus spp., Stomoxys spp., Trichodectes spp., Rhodnius spp., Ctenocephalides canis, Cimx lectularius, Ctenocephalides felis, Lucilia cuprina and the like. As Acarina, there are for example, Ornithodoros spp., Ixodes spp., Boophilus spp. and the like.

In the veterinary field, i.e., in the field of veterinary medicine, the active compounds of the present invention show an activity against parasites, in particular endoparasites and ectoparasites. The term “endoparasites” especially include helminths such as tapeworms, nematodes, and trematodes and protozoas such as coccidian. “Ectoparasites” include; typically and also preferably, arthropods, in particular, insects such as fly (biting fly and sucking fly), larva of parasitic fly, louse, public louse, bird louse, and flea, and mites of acarina such as hard tick or soft tick, sarcoptic mite, chigger mite, and bird mite.

The parasitic organisms include those described below.

from Anoplurida, for example, Haematopinus spp., Linognathus spp., Pediculus spp., Phtirus spp., Solenopotes spp.; particularly, for representative examples, Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;

from Mallophagida, Amblycerina, and Ischnocerina, for example, Trimenopon spp., Menopon spp., Trinoton spp., Bovicola spp., Werneckiella spp., Lepikentron spp., Damalina spp., Trichodectes spp., Felicola spp.; particularly, for representative examples, Bovicola bovis, Bovicola ovis, Bovicola limbata, Damalina bovis, Trichodectes canis, Felicola subrostratus, Bovicola caprae, Lepikentron ovis, Werneckiella equi;

from Diptera, Nematocerina, and Brachycerina, for example, Aedes spp., Anopheles ssp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particularly, for representative examples, Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia omata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricornis, Gasterophilus pecorum, Braula coeca;

from Siphonapterida, for example, Pulex spp., Ctenocephalides spp., Tunga spp., Xenopsylla spp., Ceratophyllus spp.; particularly, for representative examples, Ctenocephalides canis, Ctenocephalides felis, Pulex irritans, Tunga penetrans, Xenopsylla cheopis;

from Heteropterida, for example, Cimex spp., Triatoma spp., Rhodnius spp., Panstrongylus spp.;

from Blattarida, for example, Blatta orientalis, Periplaneta americana, Blattela germanica, Supella spp. (for example, Suppella longipalpa);

from Acari (Acarina), Metastigmata, and Mesostigmata, for example, Argas spp., Ornithodorus spp., Otobius spp., Ixodes spp., Amblyomma spp., Rhipicephalus (Boophilus) spp., Dermacentor spp., Haemophysalis spp., Hyalomma spp., Dermanyssus spp., Rhipicephalus spp. (original genus of heteroxenous mites), Ornithonyssus spp., Pneumonyssus spp., Raillietia spp., Pneumonyssus spp., Sternostoma spp., Varroa spp., Acarapis spp.);

particularly, for representative examples, Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma amcricanum, Amblyomma variegatum, Amblyomma maculatum, Amblyomma hcbraeum, Amblyomma cajennense, Dermanyssus gallinae, Ornithonyssus bursa, Ornithonyssus sylviarum, Varroa jacobsconi;

from Actinedida (Prostigmata), and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particularly, Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleli, Neoschonegastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

The active compounds of the present invention are also suitable for controlling arthropods, helminths and protozoas which attack an animal. The animal includes an agricultural livestock like a cow, a sheep, a goat, a horse, a pig, a donkey, a camel, a buffalo, a rabbit, a chicken, a turkey, a duck, a goose, a nursery fish, a honey bee and the like. In addition, the animal also includes a pet (i.e., companion animal) like a dog, a cat, a pet bird, an aquarium fish and the like and an animal known as a test animal like a hamster, a guinea pig, a rat, a mouse and the like.

With the control of these arthropods, helminths and/or protozoas by using the active compounds of the present invention, death ratio of the host animal is reduced, productivity (for obtaining meat, milk, wool, leather, eggs and honey, etc.) and health of the host animal are expected to be improved, and also economically more favorable and convenient breeding of the animal can be achieved.

For example, (when applicable) it is preferable that blood mixing from a host via parasites is inhibited or interrupted. In addition, control of parasite can be useful for inhibiting transfer of infectious factors.

The term “control” used in the present specification in relation to a veterinary field means that the active compounds of the present invention are effective for reducing the occurrence of parasites in the animal infected with each parasite to a harmless level. More specifically, the term “control” used in the present specification means that the active compounds of the present invention are effective for eradicating each parasite or for inhibiting its growth or proliferation.

In general, when used for an animal treatment, the compounds of the present invention can be directly applied. Preferably, the compounds of the present invention are applied as pharmaceutical compositions which may contain vehicles and/or auxiliary agents that are known in the field and pharmaceutically acceptable.

In a veterinary medicine field and livestock farming, the active compounds can be applied (administered) in various know ways, such as via enteral administration in form of a tablet, a capsule, a drink, a syrup, a granule, a paste, a bolus and a feed stuff, or a suppository; via parenteral administration based on injection (intramuscular, subcutaneous, intravenous, intraperitoneal,-etc.), implant, intranasal administration, etc.; by administration on skin in form of impregnation, liquid impregnation, spray, pouring on, spotting on, washing and powder spray; or with an aid of an molded article containing the active compounds, such as a neck tag, an ear tag, a tail tag, a leg tag, a horse rein, an identification tag, etc. The active compounds also can be prepared as shampoo, an appropriate preparation usable in aerosol, or as an unpressurized spray, for example a pump spray and a sprayer.

When used for livestock, poultry, pet and the like, the active compounds of the present invention can be prepared as a formulation containing them in an amount of 1 to 80% of weight (for example, powder, wettable preparation (WP), an emulsion, an emulsified concentrate (EC), a flowable, a homogenous solution and a suspension concentrate (SC)), and then can be applied directly or after dilution (for example, 100 to 10,000 times dilution), or they can be also applied as impregnation solution.

When used in a field of veterinary medicine, the active compounds of the present invention can be used in combination with appropriate synergists such as acaricidal agents, pesticides, anti-helminth agents or anti-protozoa agents or with other active compounds.

In the present invention, the compounds which have a pesticidal activity against the harmful pests encompassing all of the above are referred to as pesticides.

When used as pesticides, the active compounds of the present invention can be prepared in a form of common preparation. Such preparation form may includes, for example, a solution, an emulsion, wettable powder, granulated wettable powder, a suspension, powder, a foam, a paste, a tablet, a granule, an aerosol, a natural or synthetic agent impregnated with the active compounds, a microcapsule, a coating agent for seeds, a formulation equipped with a combustion device (the combustion device can be a smoke or fog cartridge, a can or a coil, etc.) and ULV (cold mist, warm mist), and the like.

These formulations may be prepared by methods known per se. For example, they can be prepared by mixing the active compounds together with spreading agents, i.e. liquid diluents or carriers; liquefied gas diluents or carriers; solid diluents or carriers, and, optionally, with surfactants i.e. emulsifiers and/or dispersants and/or foam-forming agents.

When water is used as a spreading agent, for example, organic solvents may be used as auxiliary solvents.

Liquid diluents or carriers may include: for example, aromatic hydrocarbons (e.g. xylene, toluene, alkylnaphthalene etc.), chlorinated aromatic or chlorinated aliphatic hydrocarbons (e.g. chlorobenzenes, ethylene chlorides, methylene chlorides etc.), aliphatic hydrocarbons (e.g. cyclohexanes) or paraffins (e.g. mineral oil fractions), alcohols (e.g. butanol, glycol and ethers or esters thereof, etc.), ketones (e.g. acetone, methyl ethyl ketone, methyl isobutyl ketone, cyclohexanone etc.), strong polar solvents (e.g. dimethylformamide, dimethylsulfoxide etc.), water and the like.

Liquefied gas diluents or carriers may include those present as gas at atmospheric temperature and by evaporation, for example, butane, propane, nitrogen gas, carbon dioxide, and an aerosol propellant such as halogenated hydrocarbons.

Examples of the solid diluents include ground natural minerals (for example, kaolins, clay, talc, chalk, quartz, attapulgite, montmorillonite, diatomaceous earth, etc.) and finely-ground synthetic minerals (for example, highly dispersed silicic acid, alumina and silicate, etc.) and the like.

Examples of the solid carriers for granules may include finely pulverized and sifted rocks (for example, calcite, marble, pumice, sepiolite and dolomite, etc.), synthetic granules of inorganic or organic powders, and fine granules of organic materials (for example, sawdust, coconut shells, corn cobs and tobacco stalks, etc.) and the like.

Examples of the emulsifiers and/or foam formers may include nonionic and anionic emulsifiers, for example, polyoxyethylene fatty acid esters, polyoxyethylene fatty acid alcohol ethers (for example, alkylaryl polyglycol ether), alkyl sulfonates, alkyl sulfates and aryl sulfonates, and albumin hydrolysates and the like.

Examples of the dispersants include lignin sulfite waste liquor and methylcellulose.

Binders may also be used in the formulation (powder, granule and emulsion). Examples of the binders may include carboxymethyl cellulose, natural or synthetic polymers (for example, gum arabic, polyvinyl alcohol and polyvinyl acetate, etc.).

Colorants may also be used. Examples of the colorants may include inorganic pigments (for example, iron oxide, titanium oxide and Prussian blue, etc.), organic dyes such as Alizarin dyes, azo dyes or metal phthalocyanine dyes, and further, trace elements such as salts of iron, manganese, boron, copper, cobalt, molybdenum or zinc.

In general, the formulation may include the above active components in an amount of 0.1 to 95% by weight, preferably 0.5 to 90% by weight.

The compounds of the present invention can be provided as mixtures with other active compounds such as pesticides, poison baits, sterilizing agents, acaricidal agents, nematocides, fungicides, growth regulating agents, and herbicides in a form of commercially useful formulation or an application form modified from formulation thereof.

The amount of the compounds of the present invention in commercially useful application form may vary over a broad range.

The concentration of the active compounds of the present invention for actual use may be, for example, between 0.0000001 and 100% by weight, preferably between 0.00001 and 1% by weight.

The compounds of the present invention can be used according to any common methods suitable for each application form.

The compounds of the present invention have stability that is effective for alkaline substances present on lime materials when the compounds are used against hygienic pests and other stored product pests. In addition, they exhibit excellent residual effectiveness on woods and soils.

### SYNTHETIC EXAMPLES

### Synthesis of N-{4-[5-(3,5-dichlorophenyl)-2-oxo-5-(trifluoromethyl)-1,3-oxazolin-3-yl]-2-(trifluoromethyl)benzyl}propane amide (Compound No. 1-3)

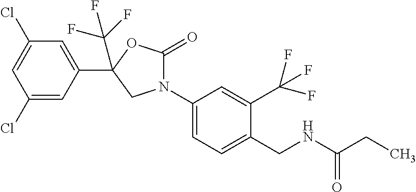

### Step 1. Synthesis of 2-(3,5-dichlorophenyl)-1,1,1-trifluoro-3-nitropropan-2-ol

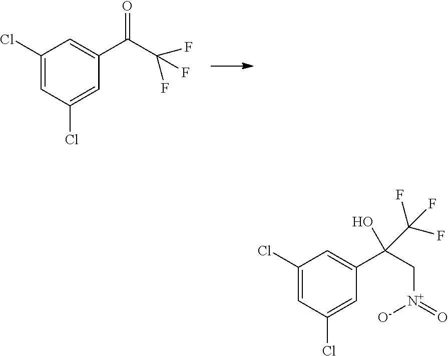

1-(3,5-Dichlorophenyl)-2,2,2-trifluoroethane (5.0 g) and potassium carbonate (2.85 g) were suspended in nitromethane (23 ml) and the mixture was stirred at room temperature for 2 hours. To the reaction solution, an aqueous solution of acetic acid (acetic acid 3 ml and water 50 ml) was added under ice cooling, followed by stirring. The reaction solution was extracted with ethyl acetate. The organic layer was washed with water and saturated brine, and dried over anhydrous magnesium sulfate. Under reduced pressure (760 to 20 mmHg), the solvent was distilled off to obtain 2-(3,5-dichlorophenyl)-1,1,1-trifluoro-3-nitropropan-2-ol (6.53 g).

¹H-NMR (CDCl₃) δ: 4.86 (1H, s), 5.01 (2H, s), 7.45-7.50 (3H, m)

### Step 2. Synthesis of 3-amino-2-(3,5-dichlorophenyl)-1,1,1-trifluoropropan-2-ol

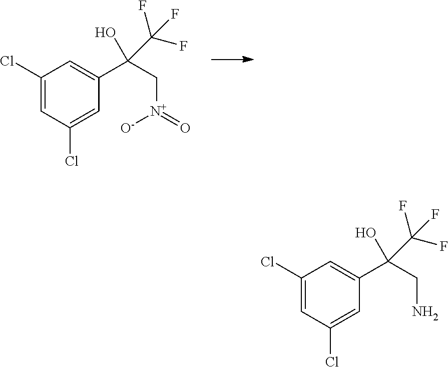

2-(3,5-Dichlorophenyl)-1,1,1-trifluoro-3-nitropropan-2-ol (0.50 g) was dissolved in ethanol (20 ml). To the solution, tin (II) chloride (1.23 g) and concentrated hydrochloric acid (2 ml) were added and the mixture was stirred at 60° C. for 4 hours. After returning to room temperature, ethyl acetate (40 ml) and water (40 ml) were added, and then potassium carbonate was added thereto under vigorous stirring until the mixture is neutralized. Celite filtration was performed. The organic layer was separated and the aqueous layer was extracted with ethyl acetate. The organic layer was combined, washed with saturated brine, and dried over magnesium sulfate. The drying agent was filtered off, and the solvent was distilled off under reduced pressure to obtain a residue. The resulting residue was purified by silica gel column chromatography to obtain 3-amino-2-(3,5-dichlorophenyl)-1,1,1-trifluoropropan-2-ol (0.21 g).

¹H-NMR (CDCl₃) δ: 2.92 (1H, d), 3.54 (1H, d), 7.39-7.44 (3H, m)

### Step 3. Synthesis of 5-(3,5-dichlorophenyl)-5-(trifluoromethyl)-1,3-oxazolin-2-one

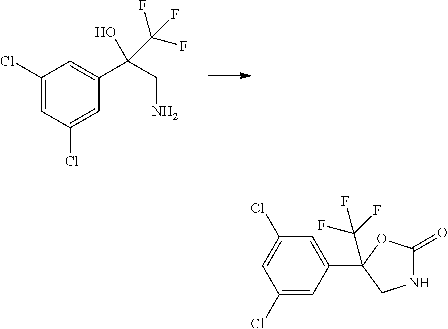

A dichloromethane (20 ml) solution of 3-amino-2-(3,5-dichlorophenyl)-1,1,1-trifluoropropan-2-ol (790 mg) was added to a saturated aqueous solution of sodium hydrogen carbonate (10 ml). Then, triphosgene (860 mg) dissolved in dichloromethane (5 ml) was added dropwise to the mixture, followed by stirring at room temperature for 90 minutes. The organic layer was washed with water, 1 N aqueous solution of hydrochloric acid and saturated brine in order, and then dried over anhydrous magnesium sulfate. The solvent was distilled off under reduced pressure to obtain 5-(3,5-dichlorophenyl)-5-(trifluoromethyl)-1,3-oxazolin-2-one (1.12 g).

¹H-NMR (CDCl₃) δ: 3.86 (1H, d), 4.24 (1H, d), 7.40 (2H, s), 7.46 (1H, s)

### Step 4. Synthesis of 4-[5-(3,5-dichlorophenyl)-2-oxo-5-(trifluoromethyl)-1,3-oxazolin-3-yl]-2-(trifluoromethyl)benzonitrile

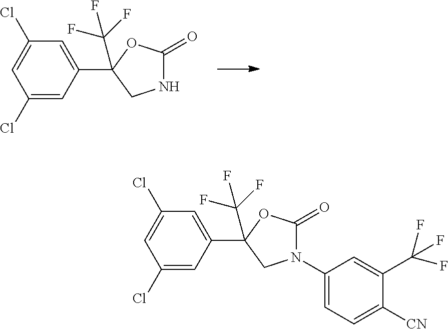

Under argon atmosphere, sodium hydride (42 mg) was added at once to a N,N-dimethylformamide (10 ml) solution of 5-(3,5-dichlorophenyl)-5-(trifluoromethyl)-1,3-oxazolin-2-one (256 mg). The mixture was stirred at room temperature for 45 minutes, and then 4-fluoro-2-(trifluoromethyl)benzonitrile (242 mg) was added, followed by stirring at room temperature for 4 hours. The reaction solution was diluted by adding water, extracted with t-butylmethyl ether, and the organic layer was dried over anhydrous magnesium sulfate. The solvent was distilled off under reduced pressure and the residue was purified by silica gel column chromatography to obtain 4-[5-(3,5-dichlorophenyl)-2-oxo-5-(trifluoromethyl)-1,3-oxazolin-3-yl]-2-(trifluoromethyl)benzonitrile (346 mg).

¹H-NMR (CDCl₃) δ: 4.38 (1H, d), 4.71 (1H, d), 7.50-7.51 (3H, m), 7.89-7.96 (3H, m)

### Step 5. Synthesis of tert-butyl{4-[5-(3,5-dichlorophenyl)-2-oxo-5-(trifluoromethyl)-1,3-oxazolin-3-yl]-2-(trifluoromethyl)benzyl}carbamate

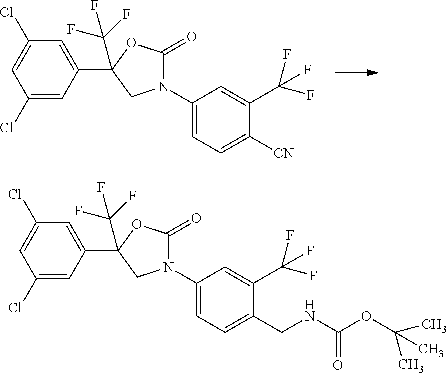

4-[5-(3,5-Dichlorophenyl)-2-oxo-5-(trifluoromethyl)-1,3-oxazolin-3-yl]-2-(trifluoromethyl)benzonitrile (346 mg), di-tert-butyl bicarbonate (0.34 ml) and nickel (II) chloride hexahydrate (176 mg) were dissolved in a mixture solution of methanol (10 ml) and 1,4-dioxane (20 ml), and then sodium borohydride (140 mg) was added in small portions under ice cooling. After stirring the mixture for 30 minutes under ice cooling, sodium borohydride (140 mg) was further added in small portions, and stirred under ice cooling for 30 minutes. The reaction solution was stirred for 1 hour at room temperature, added with diethylene triamine (1.6 ml), and stirred for 1 hour. To the reaction solution, water and ethyl acetate were added, and the mixture was extracted with ethyl acetate. The organic layer was washed with a saturated aqueous solution of sodium hydrogen carbonate, and dried over anhydrous magnesium sulfate. The solvent was distilled off under reduced pressure and the residue was purified by silica gel column chromatography to obtain tert-butyl {4-[5-(3,5-dichlorophenyl)-2-oxo-5-(trifluoromethyl)-1,3-oxazolin-3-yl]-2-(trifluoromethyl)benzyl}carbamate (360 mg).

¹H-NMR (CDCl₃) δ: 1.45 (9H, s), 4.31 (1H, d), 4.47 (2H, d), 4.64 (1H, d), 4.99 (1H, br s), 7.50 (3H, s), 7.63-7.75 (3H, m)

### Step 6. Synthesis of 3-[4-(aminomethyl)-3-(trifluoromethyl)phenyl-5-(3,5-dichlorophenyl)-5-(trifluoromethyl)-1,3-oxazolin-2-one (Compound No. 1-1)

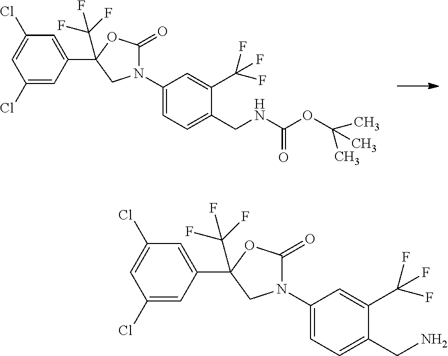

Tert-butyl {4-[5-(3,5-dichlorophenyl)-2-oxo-5-(trifluoromethyl)-1,3-oxazolin-3-yl]-2-(trifluoromethyl)benzyl}carbamate (277 mg) was dissolved in dichloromethane (20 ml), added with trifluoroacetic acid (2.1 ml), and stirred for 85 minutes at room temperature. The reaction solution was concentrated under reduced pressure, added with a saturated aqueous solution of sodium hydrogen carbonate, and extracted with t-butylmethyl ether. The organic layer was dried over anhydrous magnesium sulfate. The solvent was distilled off under reduced pressure to obtain 3-[4-(aminomethyl)-3-(trifluoromethyl)phenyl-5-(3,5-dichlorophenyl)-5-(trifluoromethyl)-1,3-oxazolin-2-one (184 mg).

¹H-NMR (CDCl₃) δ: 4.31 (1H, d), 4.55 (2H, s), 4.64 (1H, d), 7.49 (3H, s), 7.75 (3H, s)

### Step 7. Synthesis of N-{4-[5-(3,5-dichlorophenyl)-2-oxo-5-(trifluoromethyl)-1,3-oxazolin-3-yl]-2-(trifluoromethyl)benzyl}propane amide (Compound No. 1-3)

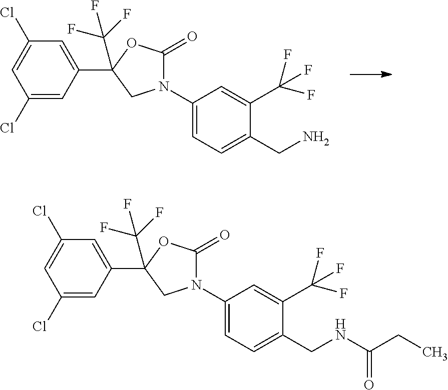

To a tetrahydrofuran (5 ml) solution of 3-[4-(aminomethyl)-3-(trifluoromethyl)phenyl-5-(3,5-dichlorophenyl)-5-(trifluoromethyl)-1,3-oxazolin-2-one (92 mg), propionic anhydride (25 mg) was added and the mixture was stirred at room temperature for 23 hours. The reaction solution was concentrated under reduced pressure and the residue was purified by silica gel column chromatography to obtain N-{4-[5-(3,5-dichlorophenyl)-2-oxo-5-(trifluoromethyl)-1,3-oxazolin-3-yl]-2-(trifluoromethyl)benzyl}propane amide (87 mg).

¹H-NMR (CDCl₃) δ: 2.01 (3H, d), 4.29 (1H, d), 4.59-4.63 (3H, m), 5.79 (1H, br s), 7.50-7.50 (3H, m), 7.65-7.68 (2H, m), 7.81 (1H, s)

### Synthesis of N-(1-{4-[2-oxo-5-(3,4,5-trichlorophenyl)-5-(trifluoromethyl)-1,3-oxazolidin-3-yl]phenyl}ethyl)propanamide (Compound No. 1-371)

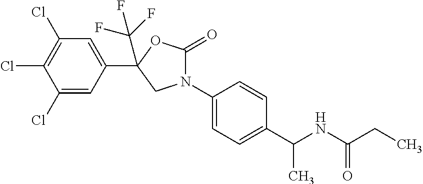

### Step 1. Synthesis of N-[1-(4-bromophenyl)ethyl]propanamide

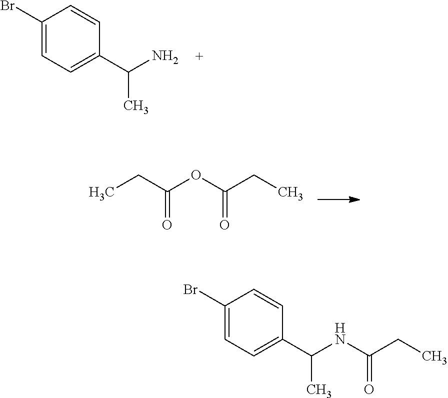

Propionic anhydride (1.86 g) was added to the solution of 1-(4-bromophenyl)ethylamine (2.38 g) and triethylamine (1.45 g) in tetrahydrofuran (50 ml), and the mixture was stirred at room temperature for 6 hours. The reaction mixture concentrated under reduced pressure, and the residue was purified by silica gel column chromatography to obtain N-[1-(4-bromophenyl)ethyl]propanamide (2.81 g).

1H-NMR (CDCl3) δ: 1.15 (3H, t), 1.46 (3H, d), 2.21 (2H, q), 5.04-5.13 (1H, m), 5.63 (1H, br s), 7.19-7.24 (2H, m), 7.44-7.47 (2H, m).

### Step 2. N-[1-{4-[2-oxo-5-(3,4,5-trichlorophenyl)-5-trifluoromethyl)-1,3-oxazolidin-3-yl]phenyl}ethyl)propanamide (Compound No. 1-371)

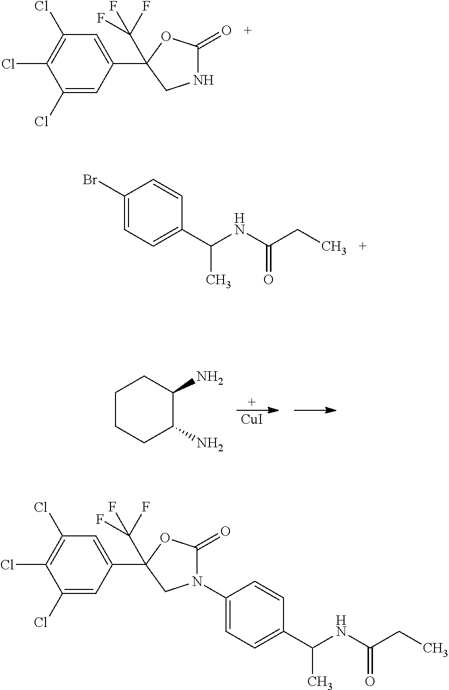

To a solution of 5-(3,4,5-trichlorophenyl)-5-(trifluoromethyl)-1,3-oxazolidin-2-one (200 mg) and N-[1-(4-bromophenyl)ethyl]propanamide (153 mg) in 1,4-dioxane (4 ml) was added trans-1,2-cyclohexanediamine (68 mg), copper(I) iodide (114 mg) and potassium carbonate (83 mg), and then heated with stirring at 110° C. for 7.5 hours under argon atmosphere. The reaction mixture was cooled to room temperature and then filtrated through Celite, and the filtrate was concentrated under reduced pressure, and the residue was purified by silica gel column chromatography to obtain N-[1-{4-[2-oxo-5-(3,4,5-trichlorophenyl)-5-trifluoromethyl)-1,3-oxazolidin-3-yl]phenyl}ethyl)propanamide (270 mg).

1H-NMR (CDCl3) δ: 1.13-1.16 (3H, m), 2.19-2.22 (2H, m), 4.26 (1H, d), 4.59 (1H, d), 5.09-5.11 (1H, m), 5.73 (1H, br s), 7.34 (2H, d), 7.45 (2H, d), 7.61 (2H, s).

In addition, each compound that obtained from the synthetic examples described herein is also indicated in the corresponding tables.

Abbreviated symbols and others described in the tables are as follows: Me: methyl, Et: ethyl, Pr: propyl, Bu: butyl, n-: normal, cyclo-: cyclo, tert-: tertiary. Cpd: compound.

In the following tables, if the compound number having a suffix “-a” or “-b”, then it means that the compound exists in at least two optical isomers at the carbon atom marked by “*”. The compound number having as suffix “-a” stands for a (S)-isomer and the compound number having a suffix “-b” stands for a R-isomer. For example compound 1-371 is the racemate, compound 1-371-a is the (S)-isomer and compound 1-371-bis the (R)-isomer.

TABLE 1

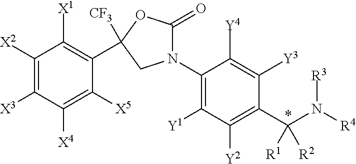

wherein X¹, X^{5.} Y¹, Y², Y⁴, R² and R³ represent hydrogen.

| Cpd. No. | X² | X³ | X⁴ | Y³ | R¹ | R⁴ |
|---|---|---|---|---|---|---|
| 1-1 | Cl | H | Cl | CF₃ | H | H |
| 1-2 | Cl | H | Cl | CF₃ | H | MeCO |
| 1-3 | Cl | H | Cl | CF₃ | H | EtCO |
| 1-4 | Cl | H | Cl | CF₃ | H | n-PrCO |
| 1-5 | Cl | H | Cl | CF₃ | H | cyclo-PrCO |
| 1-6 | Cl | H | Cl | CF₃ | H | cyclo-PrCH₂CO |
| 1-7 | Cl | H | Cl | CF₃ | H | CF₃CH₂CO |
| 1-8 | Cl | H | Cl | CF₃ | H | CH₃SCH₂CO |
| 1-9 | Cl | H | Cl | CF₃ | H | CH₃SOCH₂CO |
| 1-10 | Cl | H | Cl | CF₃ | H | CH₃SO₂CH₂CO |
| 1-11 | Cl | H | Cl | CF₃ | H | CH₃OCH₂CH₂CO |
| 1-12 | Cl | H | Cl | CF₃ | H | CH₃OCH(Me)CH₂CO |
| 1-13 | Cl | H | Cl | CF₃ | H | EtNHCO |
| 1-14 | Cl | H | Cl | CF₃ | H | tert-BuOC(═O) |
| 1-15 | Cl | Cl | Cl | CF₃ | H | H |
| 1-16 | Cl | Cl | Cl | CF₃ | H | MeCO |
| 1-17 | Cl | Cl | Cl | CF₃ | H | EtCO |
| 1-18 | Cl | Cl | Cl | CF₃ | H | n-PrCO |
| 1-19 | Cl | Cl | Cl | CF₃ | H | cyclo-PrCO |
| 1-20 | Cl | Cl | Cl | CF₃ | H | cyclo-PrCH₂CO |
| 1-21 | Cl | Cl | Cl | CF₃ | H | CF₃CH₂CO |
| 1-22 | Cl | Cl | Cl | CF₃ | H | CH₃SCH₂CO |
| 1-23 | Cl | Cl | Cl | CF₃ | H | CH₃SOCH₂CO |
| 1-24 | Cl | Cl | Cl | CF₃ | H | CH₃SO₂CH₂CO |
| 1-25 | Cl | Cl | Cl | CF₃ | H | CH₃OCH₂CH₂CO |
| 1-26 | Cl | Cl | Cl | CF₃ | H | CH₃OCH(Me)CH₂CO |
| 1-27 | Cl | Cl | Cl | CF₃ | H | EtNHCO |
| 1-28 | Cl | Cl | Cl | CF₃ | H | tert-BuOC(═O) |
| 1-29 | Cl | Cl | Cl | Cl | H | H |
| 1-30 | Cl | Cl | Cl | Cl | H | MeCO |
| 1-31 | Cl | Cl | Cl | Cl | H | EtCO |
| 1-32 | Cl | Cl | Cl | Cl | H | n-PrCO |
| 1-33 | Cl | Cl | Cl | Cl | H | cyclo-PrCO |
| 1-34 | Cl | Cl | Cl | Cl | H | cyclo-PrCH₂CO |
| 1-35 | Cl | Cl | Cl | Cl | H | CF₃CH₂CO |
| 1-36 | Cl | Cl | Cl | Cl | H | CH₃SCH₂CO |
| 1-37 | Cl | Cl | Cl | Cl | H | CH₃SOCH₂CO |
| 1-38 | Cl | Cl | Cl | Cl | H | CH₃SO₂CH₂CO |
| 1-39 | Cl | Cl | Cl | Cl | H | CH₃OCH₂CH₂CO |
| 1-40 | Cl | Cl | Cl | Cl | H | CH₃OCH(Me)CH₂CO |
| 1-41 | Cl | Cl | Cl | Cl | H | EtNHCO |
| 1-42 | Cl | Cl | Cl | Cl | H | tert-BuOC(═O) |
| 1-43 | Cl | Cl | Cl | Me | H | H |
| 1-44 | Cl | Cl | Cl | Me | H | MeCO |
| 1-45 | Cl | Cl | Cl | Me | H | EtCO |
| 1-46 | Cl | Cl | Cl | Me | H | n-PrCO |
| 1-47 | Cl | Cl | Cl | Me | H | cyclo-PrCO |
| 1-48 | Cl | Cl | Cl | Me | H | cyclo-PrCH₂CO |
| 1-49 | Cl | Cl | Cl | Me | H | CF₃CH₂CO |
| 1-50 | Cl | Cl | Cl | Me | H | CH₃SCH₂CO |
| 1-51 | Cl | Cl | Cl | Me | H | CH₃SOCH₂CO |
| 1-52 | Cl | Cl | Cl | Me | H | CH₃SO₂CH₂CO |
| 1-53 | Cl | Cl | Cl | Me | H | CH₃OCH₂CH₂CO |
| 1-54 | Cl | Cl | Cl | Me | H | CH₃OCH(Me)CH₂CO |
| 1-55 | Cl | Cl | Cl | Me | H | EtNHCO |
| 1-56 | Cl | Cl | Cl | Me | H | tert-BuOC(═O) |
| 1-57 | Cl | Cl | Cl | H | H | H |
| 1-58 | Cl | Cl | Cl | H | H | MeCO |
| 1-59 | Cl | Cl | Cl | H | H | EtCO |
| 1-60 | Cl | Cl | Cl | H | H | n-PrCO |
| 1-61 | Cl | Cl | Cl | H | H | cyclo-PrCO |
| 1-62 | Cl | Cl | Cl | H | H | cyclo-PrCH₂CO |
| 1-63 | Cl | Cl | Cl | H | H | CF₃CH₂CO |
| 1-64 | Cl | Cl | Cl | H | H | CH₃SCH₂CO |
| 1-65 | Cl | Cl | Cl | H | H | CH₃SOCH₂CO |
| 1-66 | Cl | Cl | Cl | H | H | CH₃SO₂CH₂CO |
| 1-67 | Cl | Cl | Cl | H | H | CH₃OCH₂CH₂CO |
| 1-68 | Cl | Cl | Cl | H | H | CH₃OCH(Me)CH₂CO |
| 1-69 | Cl | Cl | Cl | H | H | EtNHCO |
| 1-70 | Cl | Cl | Cl | H | H | tert-BuOC(═O) |
| 1-71 | CF₃ | H | H | CF₃ | H | H |
| 1-72 | CF₃ | H | H | CF₃ | H | MeCO |
| 1-73 | CF₃ | H | H | CF₃ | H | EtCO |
| 1-74 | CF₃ | H | H | CF₃ | H | n-PrCO |
| 1-75 | CF₃ | H | H | CF₃ | H | cyclo-PrCO |
| 1-76 | CF₃ | H | H | CF₃ | H | cyclo-PrCH₂CO |
| 1-77 | CF₃ | H | H | CF₃ | H | CF₃CH₂CO |
| 1-78 | CF₃ | H | H | CF₃ | H | CH₃SCH₂CO |
| 1-79 | CF₃ | H | H | CF₃ | H | CH₃SOCH₂CO |
| 1-80 | CF₃ | H | H | CF₃ | H | CH₃SO₂CH₂CO |
| 1-81 | CF₃ | H | H | CF₃ | H | CH₃OCH₂CH₂CO |
| 1-82 | CF₃ | H | H | CF₃ | H | CH₃OCH(Me)CH₂CO |
| 1-83 | CF₃ | H | H | CF₃ | H | EtNHCO |
| 1-84 | CF₃ | H | H | CF₃ | H | tert-BuOC(═O) |
| 1-85 | CF₃ | H | H | Cl | H | H |
| 1-86 | CF₃ | H | H | Cl | H | MeCO |
| 1-87 | CF₃ | H | H | Cl | H | EtCO |
| 1-88 | CF₃ | H | H | Cl | H | n-PrCO |
| 1-89 | CF₃ | H | H | Cl | H | cyclo-PrCO |
| 1-90 | CF₃ | H | H | Cl | H | cyclo-PrCH₂CO |
| 1-91 | CF₃ | H | H | Cl | H | CF₃CH₂CO |
| 1-92 | CF₃ | H | H | Cl | H | CH₃SCH₂CO |
| 1-93 | CF₃ | H | H | Cl | H | CH₃SOCH₂CO |
| 1-94 | CF₃ | H | H | Cl | H | CH₃SO₂CH₂CO |
| 1-95 | CF₃ | H | H | Cl | H | CH₃OCH₂CH₂CO |
| 1-96 | CF₃ | H | H | Cl | H | CH₃OCH(Me)CH₂CO |
| 1-97 | CF₃ | H | H | Cl | H | EtNHCO |
| 1-98 | CF₃ | H | H | Cl | H | tert-BuOC(═O) |
| 1-99 | CF₃ | H | H | Me | H | H |
| 1-100 | CF₃ | H | H | Me | H | MeCO |
| 1-101 | CF₃ | H | H | Me | H | EtCO |
| 1-102 | CF₃ | H | H | Me | H | n-PrCO |
| 1-103 | CF₃ | H | H | Me | H | cyclo-PrCO |
| 1-104 | CF₃ | H | H | Me | H | cyclo-PrCH₂CO |
| 1-105 | CF₃ | H | H | Me | H | CF₃CH₂CO |
| 1-106 | CF₃ | H | H | Me | H | CH₃SCH₂CO |
| 1-107 | CF₃ | H | H | Me | H | CH₃SOCH₂CO |
| 1-108 | CF₃ | H | H | Me | H | CH₃SO₂CH₂CO |
| 1-109 | CF₃ | H | H | Me | H | CH₃OCH₂CH₂CO |
| 1-110 | CF₃ | H | H | Me | H | CH₃OCH(Me)CH₂CO |
| 1-111 | CF₃ | H | H | Me | H | EtNHCO |
| 1-112 | CF₃ | H | H | Me | H | tert-BuOC(═O) |
| 1-113 | CF₃ | H | H | H | H | H |
| 1-114 | CF₃ | H | H | H | H | MeCO |
| 1-115 | CF₃ | H | H | H | H | EtCO |
| 1-116 | CF₃ | H | H | H | H | n-PrCO |
| 1-117 | CF₃ | H | H | H | H | cyclo-PrCO |
| 1-118 | CF₃ | H | H | H | H | cyclo-PrCH₂CO |
| 1-119 | CF₃ | H | H | H | H | CF₃CH₂CO |
| 1-120 | CF₃ | H | H | H | H | CH₃SCH₂CO |
| 1-121 | CF₃ | H | H | H | H | CH₃SOCH₂CO |
| 1-122 | CF₃ | H | H | H | H | CH₃SO2CH₂CO |
| 1-123 | CF₃ | H | H | H | H | CH₃OCH₂CH₂CO |
| 1-124 | CF₃ | H | H | H | H | CH₃OCH(Me)CH₂CO |
| 1-125 | CF₃ | H | H | H | H | EtNHCO |
| 1-126 | CF₃ | H | H | H | H | tert-BuOC(═O) |
| 1-127 | CF₃ | H | CF₃ | CF₃ | H | H |
| 1-128 | CF₃ | H | CF₃ | CF₃ | H | MeCO |
| 1-129 | CF₃ | H | CF₃ | CF₃ | H | EtCO |
| 1-130 | CF₃ | H | CF₃ | CF₃ | H | n-PrCO |
| 1-131 | CF₃ | H | CF₃ | CF₃ | H | cyclo-PrCO |
| 1-132 | CF₃ | H | CF₃ | CF₃ | H | cyclo-PrCH₂CO |
| 1-133 | CF₃ | H | CF₃ | CF₃ | H | CF₃CH₂CO |
| 1-134 | CF₃ | H | CF₃ | CF₃ | H | CH₃SCH₂CO |
| 1-135 | CF₃ | H | CF₃ | CF₃ | H | CH₃SOCH₂CO |
| 1-136 | CF₃ | H | CF₃ | CF₃ | H | CH₃SO2CH₂CO |
| 1-137 | CF₃ | H | CF₃ | CF₃ | H | CH₃OCH₂CH₂CO |
| 1-138 | CF₃ | H | CF₃ | CF₃ | H | CH₃OCH(Me)CH₂CO |
| 1-139 | CF₃ | H | CF₃ | CF₃ | H | EtNHCO |
| 1-140 | CF₃ | H | CF₃ | CF₃ | H | tert-BuOC(═O) |
| 1-141 | CF₃ | H | CF₃ | Cl | H | H |
| 1-142 | CF₃ | H | CF₃ | CL | H | MeCO |
| 1-143 | CF₃ | H | CF₃ | Cl | H | EtCO |
| 1-144 | CF₃ | H | CF₃ | Cl | H | n-PrCO |
| 1-145 | CF₃ | H | CF₃ | Cl | H | cyclo-PrCO |
| 1-146 | CF₃ | H | CF₃ | Cl | H | cyclo-PrCH₂CO |
| 1-147 | CF₃ | H | CF₃ | Cl | H | CF₃CH₂CO |
| 1-148 | CF₃ | H | CF₃ | Cl | H | CH₃SCH₂CO |
| 1-149 | CF₃ | H | CF₃ | Cl | H | CH₃SOCH₂CO |
| 1-150 | CF₃ | H | CF₃ | Cl | H | CH₃SO2CH₂CO |
| 1-151 | CF₃ | H | CF₃ | Cl | H | CH₃OCH₂CH₂CO |
| 1-152 | CF₃ | H | CF₃ | Cl | H | CH₃OCH(Me)CH₂CO |
| 1-153 | CF₃ | H | CF₃ | CL | H | EtNHCO |
| 1-154 | CF₃ | H | CF₃ | CL | H | tert-BuOC(═O) |
| 1-155 | CF₃ | H | CF₃ | Me | H | H |
| 1-156 | CF₃ | H | CF₃ | Me | H | MeCO |
| 1-157 | CF₃ | H | CF₃ | Me | H | EtCO |
| 1-158 | CF₃ | H | CF₃ | Me | H | n-PrCO |
| 1-159 | CF₃ | H | CF₃ | Me | H | cyclo-PrCO |
| 1-160 | CF₃ | H | CF₃ | Me | H | cyclo-PrCH₂CO |
| 1-161 | CF₃ | H | CF₃ | Me | H | CF₃CH₂CO |
| 1-162 | CF₃ | H | CF₃ | Me | H | CH₃SCH₂CO |
| 1-163 | CF₃ | H | CF₃ | Me | H | CH₃SOCH₂CO |
| 1-164 | CF₃ | H | CF₃ | Me | H | CH₃SO2CH₂CO |
| 1-165 | CF₃ | H | CF₃ | Me | H | CH₃OCH₂CH₂CO |
| 1-166 | CF₃ | H | CF₃ | Me | H | CH₃OCH(Me)CH₂CO |
| 1-167 | CF₃ | H | CF₃ | Me | H | EtNHCO |
| 1-168 | CF₃ | H | CF₃ | Me | H | tert-BuOC(═O) |
| 1-169 | CF₃ | H | CF₃ | H | H | H |
| 1-170 | CF₃ | H | CF₃ | H | H | MeCO |
| 1-171 | CF₃ | H | CF₃ | H | H | EtCO |
| 1-172 | CF₃ | H | CF₃ | H | H | n-PrCO |
| 1-173 | CF₃ | H | CF₃ | H | H | cyclo-PrCO |
| 1-174 | CF₃ | H | CF₃ | H | H | cyclo-PrCH₂CO |
| 1-175 | CF₃ | H | CF₃ | H | H | CF₃CH₂CO |
| 1-176 | CF₃ | H | CF₃ | H | H | CH₃SCH₂CO |
| 1-177 | CF₃ | H | CF₃ | H | H | CH₃SOCH₂CO |
| 1-178 | CF₃ | H | CF₃ | H | H | CH₃SO₂CH₂CO |
| 1-179 | CF₃ | H | CF₃ | H | H | CH₃OCH₂CH₂CO |
| 1-180 | CF₃ | H | CF₃ | H | H | CH₃OCH(Me)CH₂CO |
| 1-181 | CF₃ | H | CF₃ | H | H | EtNHCO |
| 1-182 | CF₃ | H | CF₃ | H | H | tert-BuOC(═O) |
| 1-183 | Cl | Cl | CF₃ | CF₃ | H | H |
| 1-184 | Cl | Cl | CF₃ | CF₃ | H | MeCO |
| 1-185 | Cl | Cl | CF₃ | CF₃ | H | EtCO |
| 1-186 | Cl | Cl | CF₃ | CF₃ | H | cyclo-PrCO |
| 1-187 | Cl | Cl | CF₃ | CF₃ | H | CH₃SCH₂CO |
| 1-188 | Cl | Cl | CF₃ | CF₃ | H | CF₃CH₂CO |
| 1-189 | Cl | Cl | CF₃ | CF₃ | H | EtNHCO |
| 1-190 | Cl | Cl | CF₃ | CF₃ | H | tert-BuOC(═O) |
| 1-191 | Cl | H | CF₃ | CF₃ | H | H |
| I -192 | Cl | H | CF₃ | CF₃ | H | MeCO |
| 1-193 | Cl | H | CF₃ | CF₃ | H | EtCO |
| 1-194 | Cl | H | CF₃ | CF₃ | H | cyclo-PrCO |
| 1-195 | Cl | H | CF₃ | CF₃ | H | CH₃SCH₂CO |
| 1-196 | Cl | H | CF₃ | CF₃ | H | CF₃CH₂CO |
| 1-197 | Cl | H | CF₃ | CF₃ | H | EtNHCO |
| 1-198 | Cl | H | CF₃ | CF₃ | H | tert-BuOC(═O) |
| 1-199 | F | H | CF₃ | CF₃ | H | H |
| 1-200 | F | H | CF₃ | CF₃ | H | MeCO |
| 1-201 | F | H | CF₃ | CF₃ | H | EtCO |
| 1-202 | F | H | CF₃ | CF₃ | H | cyclo-PrCO |
| 1-203 | F | H | CF₃ | CF₃ | H | CH₃SCH₂CO |
| 1-204 | F | H | CF₃ | CF₃ | H | CF₃CH₂CO |
| 1-205 | F | H | CF₃ | CF₃ | H | EtNHCO |
| 1-206 | F | H | CF₃ | CF₃ | H | tert-BuOC(═O) |
| 1-207 | H | F | CF₃ | CF₃ | H | H |
| 1-208 | H | F | CF₃ | CF₃ | H | MeCO |
| 1-209 | H | F | CF₃ | CF₃ | H | EtCO |
| 1-210 | H | F | CF₃ | CF₃ | H | cyclo-PrCO |
| 1-211 | H | F | CF₃ | CF₃ | H | CH₃SCH₂CO |
| 1-212 | H | F | CF₃ | CF₃ | H | CF₃CH₂CO |
| 1-213 | H | F | CF₃ | CF₃ | H | EtNHCO |
| 1-214 | H | F | CF₃ | CF₃ | H | tert-BuOC(═O) |
| 1-215 | Cl | H | Cl | Br | H | H |
| 1-216 | Cl | H | Cl | Br | H | MeCO |
| 1-217 | Cl | H | Cl | Br | H | EtCO |
| 1-218 | Cl | H | Cl | Br | H | n-PrCO |
| 1-219 | Cl | H | Cl | Br | H | cyclo-PrCO |
| 1-220 | Cl | H | Cl | Br | H | cyclo-PrCH₂CO |
| 1-221 | Cl | H | Cl | Br | H | CF₃CH₂CO |
| 1-222 | Cl | H | Cl | Br | H | CH₃SCH₂CO |
| 1-223 | Cl | H | Cl | Br | H | CH₃SOCH₂CO |
| 1-224 | Cl | H | Cl | Br | H | CH₃SO₂CH₂C0 |
| 1-225 | Cl | H | Cl | Br | H | CH₃OCH₂CH₂CO |
| 1-226 | Cl | H | Cl | Br | H | CH₃OCH(Me)CH₂CO |
| 1-227 | Cl | H | Cl | Br | H | EtNHCO |
| 1-228 | Cl | H | Cl | Br | H | tert-BuOC(═O) |
| 1-229 | Cl | Cl | Cl | Br | H | H |
| 1-230 | Cl | Cl | Cl | Br | H | Me CO |
| 1-231 | Cl | Cl | Cl | Br | H | EtCO |
| 1-232 | Cl | Cl | Cl | Br | H | n-PrCO |
| 1-233 | Cl | Cl | Cl | Br | H | cyclo-PrCO |
| 1-234 | Cl | Cl | Cl | Br | H | cyclo-PrCH₂CO |
| 1-235 | Cl | Cl | Cl | Br | H | CF₃CH₂CO |
| 1-236 | Cl | Cl | Cl | Br | H | CH₃SCH₂CO |
| 1-237 | Cl | Cl | Cl | Br | H | CH₃SOCH₂CO |
| 1-238 | Cl | Cl | Cl | Br | H | CH₃SO₂CH₂CO |
| 1-239 | Cl | Cl | Cl | Br | H | CH₃OCH₂CH₂CO |
| 1-240 | Cl | Cl | Cl | Br | H | CH₃OCH(Me)CH₂CO |
| 1-241 | Cl | Cl | Cl | Br | H | EtNHCO |
| 1-242 | Cl | Cl | Cl | Br | H | tert-BuOC(═O) |
| 1-243 | CF₃ | H | H | Br | H | H |
| 1-244 | CF₃ | H | H | Br | H | MeCO |
| 1-245 | CF₃ | H | H | Br | H | EtCO |
| 1-246 | CF₃ | H | H | Br | H | n-PrCO |
| 1-247 | CF₃ | H | H | Br | H | cyclo-PrCO |
| 1-248 | CF₃ | H | H | Br | H | cyclo-PrCH₂CO |
| 1-249 | CF₃ | H | H | Br | H | CF₃CH₂CO |
| 1-250 | CF₃ | H | H | Br | H | CH₃SCH₂CO |
| 1-251 | CF₃ | H | H | Br | H | CH₃SOCH₂CO |
| 1-252 | CF₃ | H | H | Br | H | CH₃SO₂CH₂CO |
| 1-253 | CF₃ | H | H | Br | H | CH₃OCH₂CH₂CO |
| 1-254 | CF₃ | H | H | Br | H | CH₃OCH(Me)CH₂CO |
| 1-255 | CF₃ | H | H | Br | H | EtNHCO |
| 1-256 | CF₃ | H | H | Br | H | tert-BuOC(═O) |
| 1-257 | CF₃ | H | CF₃ | Br | H | H |
| 1-258 | CF₃ | H | CF₃ | Br | H | MeCO |
| 1-259 | CF₃ | H | CF₃ | Br | H | EtCO |
| 1-260 | CF₃ | H | CF₃ | Br | H | n-PrCO |
| 1-261 | CF₃ | H | CF₃ | Br | H | cyclo-PrCO |
| 1-262 | CF₃ | H | CF₃ | Br | H | cyclo-PrCH₂CO |
| 1-263 | CF₃ | H | CF₃ | Br | H | CF₃CH₂C0 |
| 1-264 | CF₃ | H | CF₃ | Br | H | CH₃SCH₂CO |
| 1-265 | CF₃ | H | CF₃ | Br | H | CH₃SOCH₂CO |
| 1-266 | CF₃ | H | CF₃ | Br | H | CH₃SO₂CH₂CO |
| 1-267 | CF₃ | H | CF₃ | Br | H | CH₃OCH₂CH₂CO |
| 1-268 | CF₃ | H | CF₃ | Br | H | CH₃OCH(Me)CH₂CO |
| 1-269 | CF₃ | H | CF₃ | Br | H | EtNHCO |
| 1-270 | CF₃ | H | CF₃ | Br | H | tert-BuOC(═O) |
| 1-271 | Br | H | Br | CF₃ | H | H |
| 1-272 | Br | H | Br | CF₃ | H | MeCO |
| 1-273 | Br | H | Br | CF₃ | H | EtCO |
| 1-274 | Br | H | Br | CF₃ | H | n-PrCO |
| 1-275 | Br | H | Br | CF₃ | H | cyclo-PrCO |
| 1-276 | Br | H | Br | CF₃ | H | cyclo-PrCH₂CO |
| 1-277 | Br | H | Br | CF₃ | H | CF₃CH₂CO |
| 1-278 | Br | H | Br | CF₃ | H | CH₃SCH₂CO |
| 1-279 | Br | H | Br | CF₃ | H | CH₃SOCH₂CO |
| 1-280 | Br | H | Br | CF₃ | H | CH₃SO₂CH₂CO |
| 1-281 | Br | H | Br | CF₃ | H | CH₃OCH₂CH₂CO |
| 1-282 | Br | H | Br | CF₃ | H | CH₃OCH(Me)CH₂CO |
| 1-283 | Br | H | Br | CF₃ | H | EtNHCO |
| 1-284 | Br | H | Br | CF₃ | H | tert-BuOC(═O) |
| 1-285 | Br | H | Br | Cl | H | H |
| 1-286 | Br | H | Br | Cl | H | MeCO |
| 1-287 | Br | H | Br | Cl | H | EtCO |
| 1-288 | Br | H | Br | Cl | H | n-PrCO |
| 1-289 | Br | H | Br | Cl | H | cyclo-PrCO |
| 1-290 | Br | H | Br | Cl | H | cyclo-PrCH₂CO |
| 1-291 | Br | H | Br | Cl | H | CF₃CH₂CO |
| 1-292 | Br | H | Br | Cl | H | CH₃SCH₂CO |
| 1-293 | Br | H | Br | Cl | H | CH₃SOCH₂CO |
| 1-294 | Br | H | Br | Cl | H | CH₃SO₂CH₂CO |
| 1-295 | Br | H | Br | Cl | H | CH₃OCH₂CH₂CO |
| 1-296 | Br | H | Br | Cl | H | CH₃OCH(Me)CH₂CO |
| 1-297 | Br | H | Br | Cl | H | EtNHCO |
| 1-298 | Br | H | Br | Cl | H | tert-BuOC(═O) |
| 1-299 | Br | H | Br | Br | H | H |
| 1-300 | Br | H | Br | Br | H | MeCO |
| 1-301 | Br | H | Br | Br | H | EtCO |
| 1-302 | Br | H | Br | Br | H | n-PrCO |
| 1-303 | Br | H | Br | Br | H | cyclo-PrCO |
| 1-304 | Br | H | Br | Br | H | cyclo-PrCH₂CO |
| 1-305 | Br | H | Br | Br | H | CF₃CH₂CO |
| 1-306 | Br | H | Br | Br | H | CH₃SCH₂CO |
| 1-307 | Br | H | Br | Br | H | CH₃SOCH₂CO |
| 1-308 | Br | H | Br | Br | H | CH₃SO₂CH₂CO |
| 1-309 | Br | H | Br | Br | H | CH₃OCH₂CH₂CO |
| 1-310 | Br | H | Br | Br | H | CH₃OCH(Me)CH₂CO |
| 1-311 | Br | H | Br | Br | H | EtNHCO |
| 1-312 | Br | H | Br | Br | H | tert-BuOC(═O) |
| 1-313 | Br | H | Br | Me | H | H |
| 1-314 | Br | H | Br | Me | H | MeCO |
| 1-315 | Br | H | Br | Me | H | EtCO |
| 1-316 | Br | H | Br | Me | H | n-PrCO |
| 1-317 | Br | H | Br | Me | H | cyclo-PrCO |
| 1-318 | Br | H | Br | Me | H | cyclo-PrCH₂CO |
| 1-319 | Br | II | Br | Me | H | CF₃CH₂CO |
| 1-320 | Br | H | Br | Me | H | CH₃SCH₂CO |
| 1-321 | Br | H | Br | Me | H | CH₃SOCH₂CO |
| 1-322 | Br | H | Br | Me | H | CH₃SO₂CH₂CO |
| 1-323 | Br | H | Br | Me | H | CH₃OCH₂CH₂CO |
| 1-324 | Br | H | Br | Me | H | CH₃OCH(Me)CH₂CO |
| 1-325 | Br | H | Br | Me | H | EtNHCO |
| 1-326 | Br | H | Br | Me | H | tert-BuOC(═O) |
| 1-327 | Br | H | Br | H | H | H |
| 1-328 | Br | H | Br | H | H | MeCO |
| 1-329 | Br | H | Br | H | H | EtCO |
| 1-330 | Br | H | Br | H | H | n-PrCO |
| 1-331 | Br | H | Br | H | H | cyclo-PrCO |
| 1-332 | Br | H | Br | H | H | cyclo-PrCH₂CO |
| 1-333 | Br | H | Br | H | H | CF₃CH₂CO |
| 1-334 | Br | H | Br | H | H | CH₃SCH₂CO |
| 1-335 | Br | H | Br | H | H | CH₃SOCH₂CO |
| 1-336 | Br | H | Br | H | H | CH₃SO₂CH₂CO |
| 1-337 | Br | H | Br | H | H | CH₃OCH₂CH₂CO |
| 1-338 | Br | H | Br | H | H | CH₃OCH(Me)CH₂CO |
| 1-339 | Br | H | Br | H | H | EtNHCO |
| 1-340 | Br | H | Br | H | H | tert-BuOC(═O) |
| 1-341 | Cl | H | Cl | H | Me | H |
| 1-342 | Cl | H | Cl | H | Me | MeCO |
| 1-343 | Cl | H | Cl | H | Me | EtCO |
| 1-344 | Cl | H | Cl | H | Me | n-PrCO |
| 1-345 | Cl | H | Cl | H | Me | cyclo-PrCO |
| 1-345-a | Cl | H | Cl | H | Me | cyclo-PrCO |
| 1-346 | Cl | H | Cl | H | Me | cyclo-PrCH₂CO |
| 1-347 | Cl | H | Cl | H | Me | CF₃CH₂CO |
| 1-348 | Cl | H | Cl | H | Me | CH₃SCH₂CO |
| 1-349 | Cl | H | Cl | H | Me | CH₃SOCH₂CO |
| 1-350 | Cl | H | Cl | H | Me | CH₃SO₂CH₂CO |
| 1-351 | Cl | H | Cl | H | Me | CH₃OCH₂CH₂CO |
| 1-352 | Cl | H | Cl | H | Me | CH₃OCH(Me)CH₂CO |
| 1-353 | Cl | H | Cl | H | Me | EtNHCO |
| 1-354 | Cl | H | Cl | H | Me | tert-BuOC(═O) |
| 1-355 | Br | H | Br | H | Me | H |
| 1-356 | Br | H | Br | H | Me | MeCO |
| 1-357 | Br | H | Br | H | Me | EtCO |
| 1-358 | Br | H | Br | H | Me | n-PrCO |
| 1-359 | Br | H | Br | H | Me | cyclo-PrCO |
| 1-360 | Br | H | Br | H | Me | cyclo-PrCH₂CO |
| 1-361 | Br | H | Br | H | Me | CF₃CH₂CO |
| 1-362 | Br | H | Br | H | Me | CH₃SCH₂CO |
| 1-363 | Br | H | Br | H | Me | CH₃SOCH₂CO |
| 1-364 | Br | H | Br | H | Me | CH₃SO₂CH₂CO |
| 1-365 | Br | H | Br | H | Me | CH₃OCH₂CH₂CO |
| 1-366 | Br | H | Br | H | Me | CH₃OCH(Me)CH₂CO |
| 1-367 | Br | H | Br | H | Me | EtNHCO |
| 1-368 | Br | H | Br | H | Me | tert-BuOC(═O) |
| 1-369 | Cl | Cl | Cl | H | Me | H |
| 1-370 | Cl | Cl | Cl | H | Me | MeCO |
| 1-370-a | Cl | Cl | Cl | H | Me | MeCO |
| 1-371 | Cl | Cl | Cl | H | Me | EtCO |
| 1-371-a | Cl | Cl | Cl | H | Me | EtCO |
| 1-371-b | Cl | Cl | Cl | H | Me | EtCO |
| 1-372 | Cl | Cl | Cl | H | Me | n-PrCO |
| 1-373 | Cl | Cl | Cl | H | Me | cyclo-PrCO |
| 1-373-a | Cl | Cl | Cl | H | Me | cyclo-PrCO |
| 1-374 | Cl | Cl | Cl | H | Me | cyclo-PrCH₂CO |
| 1-374-a | Cl | Cl | Cl | H | Me | cyclo-PrCH₂CO |
| 1-375 | Cl | Cl | Cl | H | Me | CF₃CH₂CO |
| 1-375-a | Cl | Cl | Cl | H | Me | CF₃CH₂CO |
| 1-376 | Cl | Cl | Cl | H | Me | CH₃SCH₂CO |
| 1-377 | Cl | Cl | Cl | H | Me | CH₃SOCH₂CO |
| 1-378 | Cl | Cl | Cl | H | Me | CH₃SO₂CH₂CO |
| 1-379 | Cl | Cl | Cl | H | Me | CH₃OCH₂CH₂CO |
| 1-379-a | Cl | Cl | Cl | H | Me | CH₃OCH₂CH₂CO |
| 1-380 | Cl | Cl | Cl | H | Me | CH₃OCH(Me)CH₂CO |
| 1-381 | Cl | Cl | Cl | H | Me | EtNHCO |
| 1-382 | Cl | Cl | Cl | H | Me | tert-BuOC(═O) |
| 1-383 | CF₃ | H | H | H | Me | H |
| 1-384 | CF₃ | H | H | H | Me | MeCO |
| I -385 | CF₃ | H | H | H | Me | EtCO |
| 1-386 | CF₃ | H | H | H | Me | n-PrCO |
| 1-387 | CF₃ | H | H | H | Me | cyclo-PrCO |
| 1-388 | CF₃ | H | H | H | Me | cyclo-PrCH₂CO |
| 1-389 | CF₃ | H | H | H | Me | CF₃CH₂CO |
| 1-390 | CF₃ | H | H | H | Me | CH₃SCH₂CO |
| 1-391 | CF₃ | H | H | H | Me | CH₃SOCH₂CO |
| 1-392 | CF₃ | H | H | H | Me | CH₃SO₂CH₂CO |
| 1-393 | CF₃ | H | H | H | Me | CH₃OCH₂CH₂CO |
| 1-394 | CF₃ | H | H | H | Me | CH₃OCH(Me)CH₂CO |
| 1-395 | CF₃ | H | H | H | Me | EtNHCO |
| 1-396 | CF₃ | H | H | H | Me | tert-BuOC(═O) |
| 1-397 | CF₃ | H | CF₃ | H | Me | H |
| 1-398 | CF₃ | H | CF₃ | H | Me | MeCO |
| 1-398-a | CF₃ | H | CF₃ | H | Me | MeCO |
| 1-399 | CF₃ | H | CF₃ | H | Me | EtCO |
| 1-399-a | CF₃ | H | CF₃ | H | Me | EtCO |
| 1-399-b | CF₃ | H | CF₃ | H | Me | EtCO |
| 1-400 | CF₃ | H | CF₃ | H | Me | n-PrCO |
| 1-401 | CF₃ | H | CF₃ | H | Me | cyclo-PrCO |
| 1-401-a | CF₃ | H | CF₃ | H | Me | cyclo-PrCO |
| 1-402 | CF₃ | H | CF₃ | H | Me | cyclo-PrCH₂CO |
| 1-402-a | CF₃ | H | CF₃ | H | Me | cyclo-PrCH₂CO |
| 1-403 | CF₃ | H | CF₃ | H | Me | CF₃CH₂CO |
| 1-403-a | CF₃ | H | CF₃ | H | Me | CF₃CH₂CO |
| 1-404 | CF₃ | H | CF₃ | H | Me | CH₃SCH₂CO |
| 1-405 | CF₃ | H | CF₃ | H | Me | CH₃SOCH₂CO |
| 1-406 | CF₃ | H | CF₃ | H | Me | CH₃SO₂CH₂CO |
| 1-407 | CF₃ | H | CF₃ | H | Me | CH₃OCH₂CH₂CO |
| 1-407-a | CF₃ | H | CF₃ | H | Me | CH₃OCH₂CH₂CO |
| 1-408 | CF₃ | H | CF₃ | H | Me | CH₃OCH(Me)CH₂CO |
| 1-409 | CF₃ | H | CF₃ | H | Me | EtNHCO |
| 1-410 | CF₃ | H | CF₃ | H | Me | tert-BuOC(═O) |
| 1-411 | Cl | Cl | CF₃ | H | Me | H |
| 1-412 | Cl | Cl | CF₃ | H | Me | MeCO |
| 1-413 | Cl | Cl | CF₃ | H | Me | EtCO |
| 1-414 | Cl | Cl | CF₃ | H | Me | cyclo-PrCO |
| 1-415 | Cl | Cl | CF₃ | H | Me | CH₃SCH₂CO |
| 1-416 | Cl | Cl | CF₃ | H | Me | CF₃CH₂CO |
| 1-417 | Cl | Cl | CF₃ | H | Me | EtNHCO |
| 1-418 | Cl | Cl | CF₃ | H | Me | tert-BuOC(═O) |
| 1-419 | Cl | H | CF₃ | H | Me | H |
| 1-420 | Cl | H | CF₃ | H | Me | MeCO |
| 1-421 | Cl | H | CF₃ | H | Me | EtCO |
| 1-422 | Cl | H | CF₃ | H | Me | cyclo-PrCO |
| 1-423 | Cl | H | CF₃ | H | Me | CH₃SCH₂CO |
| 1-424 | Cl | H | CF₃ | H | Me | CF₃CH₂CO |
| 1-425 | Cl | H | CF₃ | H | Me | EtNHCO |
| 1-426 | Cl | H | CF₃ | H | Me | tert-BuOC(═O) |
| 1-427 | F | H | CF₃ | H | Me | H |
| 1-428 | F | H | CF₃ | H | Me | MeCO |
| 1-429 | F | H | CF₃ | H | Me | EtCO |
| 1-430 | F | H | CF₃ | H | Me | cyclo-PrCO |
| 1-431 | F | H | CF₃ | H | Me | CH₃SCH₂CO |
| 1-432 | F | H | CF₃ | H | Me | CF₃CH₂CO |
| 1-433 | F | H | CF₃ | H | Me | EtNHCO |
| 1-434 | F | H | CF₃ | H | Me | tert-BuOC(═O) |
| 1-435 | H | F | CF₃ | H | Me | H |
| 1-436 | H | F | CF₃ | H | Me | MeCO |
| 1-437 | H | F | CF₃ | H | Me | EtCO |
| 1-438 | H | F | CF₃ | H | Me | cyclo-PrCO |
| 1-439 | H | F | CF₃ | H | Me | CH₃SCH₂CO |
| 1-440 | H | F | CF₃ | H | Me | CF₃CH₂CO |
| 1-441 | H | F | CF₃ | H | Me | EtNHCO |
| 1-442 | H | F | CF₃ | H | Me | tert-BuOC(═O) |
| 1-443 | OCF₃ | H | H | CF₃ | H | EtCO |
| 1-444 | SCF₃ | H | H | CF₃ | H | EtCO |
| 1-445 | SOCF₃ | H | H | CF₃ | H | EtCO |
| 1-446 | SO₂CF₃ | H | H | CF₃ | H | EtCO |

TABLE 2

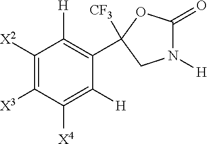

| Cpd. No. | X² | X³ | X⁴ |
|---|---|---|---|
| 2-1 | Cl | H | Cl |
| 2-2 | Cl | Cl | Cl |
| 2-3 | CF₃ | H | H |
| 2-4 | CF₃ | H | CF₃ |
| 2-5 | Cl | Cl | CF₃ |
| 2-6 | Cl | H | CF₃ |
| 2-7 | F | H | CF₃ |
| 2-8 | H | F | CF₃ |
| 2-9 | Br | H | Br |
| 2-10 | OCF₃ | H | H |
| 2-11 | SCF₃ | H | H |
| 2-12 | SOCF₃ | H | H |
| 2-13 | SO₂CF₃ | H | H |

TABLE 3

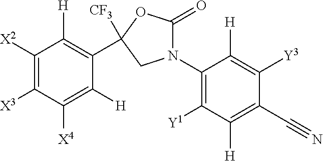

| Cpd No. | X² | X³ | X⁴ | Y³ |
|---|---|---|---|---|
| 3-1 | Cl | H | Cl | CF₃ |
| 3-2 | Cl | Cl | Cl | CF₃ |
| 3-3 | Cl | Cl | Cl | Cl |
| 3-4 | Cl | Cl | Cl | Me |
| 3-5 | Cl | Cl | Cl | H |
| 3-6 | CF₃ | H | H | CF₃ |
| 3-7 | CF₃ | H | H | Cl |
| 3-8 | CF₃ | H | H | Me |
| 3-9 | CF₃ | H | H | H |
| 3-10 | CF₃ | H | CF₃ | CF₃ |
| 3-11 | CF₃ | H | CF₃ | Cl |
| 3-12 | CF₃ | H | CF₃ | Me |
| 3-13 | CF₃ | H | CF₃ | H |
| 3-14 | Cl | Cl | CF₃ | CF₃ |
| 3-15 | Cl | H | CF₃ | CF₃ |
| 3-16 | F | H | CF₃ | CF₃ |
| 3-17 | H | F | CF₃ | CF₃ |
| 3-18 | Cl | H | Cl | Br |
| 3-19 | Cl | Cl | Cl | Br |
| 3-20 | CF₃ | H | H | Br |
| 3-21 | CF₃ | H | CF₃ | Br |
| 3-22 | Br | H | Br | CF₃ |
| 3-23 | Br | H | Br | Cl |
| 3-24 | Br | H | Br | Br |
| 3-25 | Br | H | Br | Me |
| 3-26 | Br | H | Br | H |
| 3-27 | OCF₃ | H | H | CF₃ |
| 3-28 | SCF₃ | H | H | CF₃ |
| 3-29 | SOCF₃ | H | H | CF₃ |
| 3-30 | SO₂CF₃ | H | H | CF₃ |

TABLE 4

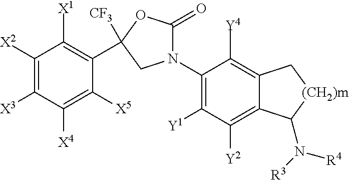

wherein X¹, X⁵, Y¹, Y², Y⁴ and R³ represent hydrogen

| Cpd. No. | X² | X³ | X⁴ | R⁴ | m |
|---|---|---|---|---|---|
| 4-1 | Cl | H | Cl | H | 1 |
| 4-2 | Cl | H | Cl | MeCO | 1 |
| 4-3 | Cl | H | Cl | EtCO | 1 |
| 4-4 | Cl | H | Cl | n-PrCO | 1 |
| 4-5 | Cl | H | Cl | cyclo-PrCO | 1 |
| 4-6 | Cl | H | Cl | cyclo-PrCH₂CO | 1 |
| 4-7 | Cl | H | Cl | CF₃CH₂CO | 1 |
| 4-8 | Cl | H | Cl | CH₃SCH₂CO | 1 |
| 4-9 | Cl | H | Cl | CH₃SOCH₂CO | 1 |
| 4-10 | Cl | H | Cl | CH₃SO₂CH₂CO | 1 |
| 4-11 | Cl | H | Cl | CH₃OCH₂CH₂CO | 1 |
| 4-12 | Cl | H | Cl | CH₃OCH(Me)CH₂CO | 1 |
| 4-13 | Cl | H | Cl | EtNHCO | 1 |
| 4-14 | Cl | H | Cl | tert-BuOC(═O) | 1 |
| 4-15 | Br | H | Br | H | 1 |
| 4-16 | Br | H | Br | MeCO | 1 |
| 4-17 | Br | H | Br | EtCO | 1 |
| 4-18 | Br | H | Br | n-PrCO | 1 |
| 4-19 | Br | H | Br | cyclo-PrCO | 1 |
| 4-20 | Br | H | Br | cyclo-PrCH₂CO | 1 |
| 4-21 | Br | H | Br | CF₃CH₂CO | 1 |
| 4-22 | Br | H | Br | CH₃SCH₂CO | 1 |
| 4-23 | Br | H | Br | CH₃SOCH₂CO | 1 |
| 4-24 | Br | H | Br | CH₃SO₂CH₂CO | 1 |
| 4-25 | Br | H | Br | CH₃OCH₂CH₂CO | 1 |
| 4-26 | Br | H | Br | CH₃OCH(Me)CH₂CO | 1 |
| 4-27 | Br | H | Br | EtNHCO | 1 |
| 4-28 | Br | H | Br | tert-BuOC(═O) | 1 |
| 4-29 | Cl | Cl | Cl | H | 1 |
| 4-30 | Cl | Cl | Cl | MeCO | 1 |
| 4-31 | Cl | Cl | Cl | EtCO | 1 |
| 4-32 | Cl | Cl | Cl | n-PrCO | 1 |
| 4-33 | Cl | Cl | Cl | cyclo-PrCO | 1 |
| 4-34 | Cl | Cl | Cl | cyclo-PrCH₂CO | 1 |
| 4-35 | Cl | Cl | Cl | CF₃CH₂CO | 1 |
| 4-36 | Cl | Cl | Cl | CH₃SCH₂CO | 1 |
| 4-37 | Cl | Cl | Cl | CH₃SOCH₂CO | 1 |
| 4-38 | Cl | Cl | Cl | CH₃SO₂CH₂CO | 1 |
| 4-39 | Cl | Cl | Cl | CH₃OCH₂CH₂CO | 1 |
| 4-40 | Cl | Cl | Cl | CH₃OCH(Me)CH₂CO | 1 |
| 4-41 | Cl | Cl | Cl | EtNHCO | 1 |
| 4-42 | Cl | Cl | Cl | tert-BuOC(═O) | 1 |
| 4-43 | CF₃ | H | H | H | 1 |
| 4-44 | CF₃ | H | H | MeCO | 1 |
| 4-45 | CF₃ | H | H | EtCO | 1 |
| 4-46 | CF₃ | H | H | n-PrCO | 1 |
| 4-47 | CF₃ | H | H | cyclo-PrCO | 1 |
| 4-48 | CF₃ | H | H | cyclo-PrCH₂CO | 1 |
| 4-49 | CF₃ | H | H | CF₃CH₂CO | 1 |
| 4-50 | CF₃ | H | H | CH₃SCH₂CO | 1 |
| 4-51 | CF₃ | H | H | CH₃SOCH₂CO | 1 |
| 4-52 | CF₃ | H | H | CH₃SO₂CH₂CO | 1 |
| 4-53 | CF₃ | H | H | CH₃OCH₂CH₂CO | 1 |
| 4-54 | CF₃ | H | H | CH₃OCH(Me)CH₂CO | 1 |
| 4-55 | CF₃ | H | H | EtNHCO | 1 |
| 4-56 | CF₃ | H | H | tert-BuOC(═O) | 1 |
| 4-57 | CF₃ | H | CF₃ | H | 1 |
| 4-58 | CF₃ | H | CF₃ | MeCO | 1 |
| 4-59 | CF₃ | H | CF₃ | EtCO | 1 |
| 4-60 | CF₃ | H | CF₃ | n-PrCO | 1 |
| 4-61 | CF₃ | H | CF₃ | cyclo-PrCO | 1 |
| 4-62 | CF₃ | H | CF₃ | cyclo-PrCH₂CO | 1 |
| 4-63 | CF₃ | H | CF₃ | CF₃CH₂CO | 1 |
| 4-64 | CF₃ | H | CF₃ | CH₃SCH₂CO | 1 |
| 4-65 | CF₃ | H | CF₃ | CH₃SOCH₂CO | 1 |
| 4-66 | CF₃ | H | CF₃ | CH₃SO₂CH₂CO | 1 |
| 4-67 | CF₃ | H | CF₃ | CH₃OCH₂CH₂CO | 1 |
| 4-68 | CF₃ | H | CF₃ | CH₃OCH(Me)CH₂CO | 1 |
| 4-69 | CF₃ | H | CF₃ | EtNHCO | 1 |
| 4-70 | CF₃ | H | CF₃ | tert-BuOC(═O) | 1 |
| 4-71 | Cl | Cl | CF₃ | H | 1 |
| 4-72 | Cl | Cl | CF₃ | MeCO | 1 |
| 4-73 | Cl | Cl | CF₃ | EtCO | 1 |
| 4-74 | Cl | Cl | CF₃ | cyclo-PrCO | 1 |
| 4-75 | Cl | Cl | CF₃ | CH₃SCH₂CO | 1 |
| 4-76 | Cl | Cl | CF₃ | CF₃CH₂CO | 1 |
| 4-77 | Cl | Cl | CF₃ | EtNHCO | 1 |
| 4-78 | Cl | Cl | CF₃ | tert-BuOC(═O) | 1 |
| 4-79 | Cl | H | CF₃ | H | 1 |
| 4-80 | Cl | H | CF₃ | MeCO | 1 |
| 4-81 | Cl | H | CF₃ | EtCO | 1 |
| 4-82 | Cl | H | CF₃ | cyclo-PrCO | 1 |
| 4-83 | Cl | H | CF₃ | CH₃SCH₂CO | 1 |
| 4-84 | Cl | H | CF₃ | CF₃CH₂CO | 1 |
| 4-85 | Cl | H | CF₃ | EtNHCO | 1 |
| 4-86 | Cl | H | CF₃ | tert-BuOC(═O) | 1 |
| 4-87 | F | H | CF₃ | H | 1 |
| 4-88 | F | H | CF₃ | MeCO | 1 |
| 4-89 | F | H | CF₃ | EtCO | 1 |
| 4-90 | F | H | CF₃ | cyclo-PrCO | 1 |
| 4-91 | F | H | CF₃ | CH₃SCH₂CO | 1 |
| 4-92 | F | H | CF₃ | CF₃CH₂CO | 1 |
| 4-93 | F | H | CF₃ | EtNHCO | 1 |
| 4-94 | F | H | CF₃ | tert-BuOC(═O) | 1 |
| 4-95 | H | F | CF₃ | H | 1 |
| 4-96 | H | F | CF₃ | MeCO | 1 |
| 4-97 | H | F | CF₃ | EtCO | 1 |
| 4-98 | H | F | CF₃ | cyclo-PrCO | 1 |
| 4-99 | H | F | CF₃ | CH₃SCH₂CO | 1 |
| 4-100 | H | F | CF₃ | CF₃CH₂C0 | 1 |
| 4-101 | H | F | CF₃ | EtNHCO | 1 |
| 4-102 | H | F | CF₃ | tert-BuOC(═O) | 1 |
| 4-103 | Cl | H | Cl | H | 2 |
| 4-104 | Cl | H | Cl | MeCO | 2 |
| 4-105 | Cl | H | Cl | EtCO | 2 |
| 4-106 | Cl | H | Cl | n-PrCO | 2 |
| 4-107 | Cl | H | Cl | cyclo-PrCO | 2 |
| 4-108 | Cl | H | Cl | cyclo-PrCH₂CO | 2 |
| 4-109 | Cl | H | Cl | CF₃CH₂CO | 2 |
| 4-110 | Cl | H | Cl | CH₃SCH₂CO | 2 |
| 4-111 | Cl | H | Cl | CH₃SOCH₂CO | 2 |
| 4-112 | Cl | H | Cl | CH₃SO₂CH₂CO | 2 |
| 4-113 | Cl | H | Cl | CH₃OCH₂CH₂CO | 2 |
| 4-114 | Cl | H | Cl | CH₃OCH(Me)CH₂CO | 2 |
| 4-115 | Cl | H | Cl | EtNHCO | 2 |
| 4-116 | Cl | H | Cl | tert-BuOC(═0) | 2 |
| 4-117 | Br | H | Br | H | 2 |
| 4-118 | Br | H | Br | MeCO | 2 |
| 4-119 | Br | H | Br | EtCO | 2 |
| 4-120 | Br | H | Br | n-PrCO | 2 |
| 4-121 | Br | H | Br | cyclo-PrCO | 2 |
| 4-122 | Br | H | Br | cyclo-PrCH₂CO | 2 |
| 4-123 | Br | H | Br | CF₃CH₂CO | 2 |
| 4-124 | Br | H | Br | CH₃SCH₂CO | 2 |
| 4-125 | Br | H | Br | CH₃SOCH₂C0 | 2 |
| 4-126 | Br | H | Br | CH₃SO₂CH₂CO | 2 |
| 4-127 | Br | H | Br | CH₃OCH₂CH₂CO | 2 |
| 4-128 | Br | H | Br | CH₃OCH(Me)CH₂CO | 2 |
| 4-129 | Br | H | Br | EtNHCO | 2 |
| 4-130 | Br | H | Br | tert-BuOC(═O) | 2 |
| 4-131 | Cl | Cl | Cl | H | 2 |
| 4-132 | Cl | Cl | Cl | MeCO | 2 |
| 4-133 | Cl | Cl | Cl | EtCO | 2 |
| 4-134 | Cl | Cl | Cl | n-PrCO | 2 |
| 4-135 | Cl | Cl | Cl | cyclo-PrCO | 2 |
| 4-136 | Cl | Cl | Cl | cyclo-PrCH₂CO | 2 |
| 4-137 | Cl | Cl | Cl | CF₃CH₂CO | 2 |
| 4-138 | Cl | Cl | Cl | CH₃SCH₂CO | 2 |
| 4-139 | Cl | Cl | Cl | CH₃SOCH₂CO | 2 |
| 4-140 | Cl | Cl | Cl | CH₃SO₂CH₂CO | 2 |
| 4-141 | Cl | Cl | Cl | CH₃OCH₂CH₂CO | 2 |
| 4-142 | Cl | Cl | Cl | CH₃OCH(Me)CH₂CO | 2 |
| 4-143 | Cl | Cl | Cl | EtNHCO | 2 |
| 4-144 | Cl | Cl | Cl | tert-BuOC(═O) | 2 |
| 4-145 | CF₃ | H | H | H | 2 |
| 4-146 | CF₃ | H | H | MeCO | 2 |
| 4-147 | CF₃ | H | H | EtCO | 2 |
| 4-148 | CF₃ | H | H | n-PrCO | 2 |
| 4-149 | CF₃ | H | H | cyclo-PrCO | 2 |
| 4-150 | CF₃ | H | H | cyclo-PrCH₂CO | 2 |
| 4-151 | CF₃ | H | H | CF₃CH₂CO | 2 |
| 4-152 | CF₃ | H | H | CH₃SCH₂CO | 2 |
| 4-153 | CF₃ | H | H | CH₃SOCH₂CO | 2 |
| 4-154 | CF₃ | H | H | CH₃SO₂CH₂CO | 2 |
| 4-155 | CF₃ | H | H | CH₃OCH₂CH₂CO | 2 |
| 4-156 | CF₃ | H | H | CH₃OCH(Me)CH₂CO | 2 |
| 4-157 | CF₃ | H | H | EtNHCO | 2 |
| 4-158 | CF₃ | H | H | tert-BuOC(═O) | 2 |
| 4-159 | CF₃ | H | CF₃ | H | 2 |
| 4-160 | CF₃ | H | CF₃ | MeCO | 2 |
| 4-161 | CF₃ | H | CF₃ | EtCO | 2 |
| 4-162 | CF₃ | H | CF₃ | n-PrCO | 2 |
| 4-163 | CF₃ | H | CF₃ | cyclo-PrCO | 2 |
| 4-164 | CF₃ | H | CF₃ | cyclo-PrCH₂CO | 2 |
| 4-165 | CF₃ | H | CF₃ | CF₃CH₂CO | 2 |
| 4-166 | CF₃ | H | CF₃ | CH₃SCH₂CO | 2 |
| 4-167 | CF₃ | H | CF₃ | CH₃SOCH₂CO | 2 |
| 4-168 | CF₃ | H | CF₃ | CH₃SO₂CH₂CO | 2 |
| 4-169 | CF₃ | H | CF₃ | CH₃OCH₂CH₂CO | 2 |
| 4-170 | CF₃ | H | CF₃ | CH₃OCH(Me)CH₂CO | 2 |
| 4-171 | CF₃ | H | CF₃ | EtNHCO | 2 |
| 4-172 | CF₃ | H | CF₃ | tert-BuOC(═O) | 2 |
| 4-173 | Cl | Cl | CF₃ | H | 2 |
| 4-174 | Cl | Cl | CF₃ | MeCO | 2 |
| 4-175 | Cl | Cl | CF₃ | EtCO | 2 |
| 4-176 | Cl | Cl | CF₃ | cyclo-PrCO | 2 |
| 4-177 | Cl | Cl | CF₃ | CH₃SCH₂CO | 2 |
| 4-178 | Cl | Cl | CF₃ | CF₃CH₂CO | 2 |
| 4-179 | Cl | Cl | CF₃ | EtNHCO | 2 |
| 4-180 | Cl | Cl | CF₃ | tert-BuOC(═O) | 2 |
| 4-181 | Cl | H | CF₃ | H | 2 |
| 4-182 | Cl | H | CF₃ | MeCO | 2 |
| 4-183 | Cl | H | CF₃ | EtCO | 2 |
| 4-184 | Cl | H | CF₃ | cyclo-PrCO | 2 |
| 4-185 | Cl | H | CF₃ | CH₃SCH₂CO | 2 |
| 4-186 | Cl | H | CF₃ | CF₃CH₂CO | 2 |
| 4-187 | Cl | H | CF₃ | EtNHCO | 2 |
| 4-188 | Cl | H | CF₃ | tert-BuOC(═O) | 2 |
| 4-189 | F | H | CF₃ | H | 2 |
| 4-190 | F | H | CF₃ | MeCO | 2 |
| 4-191 | F | H | CF₃ | EtCO | 2 |
| 4-192 | F | H | CF₃ | cyclo-PrCO | 2 |
| 4-193 | F | H | CF₃ | CH₃SCH₂CO | 2 |
| 4-194 | F | H | CF₃ | CF₃CH₂CO | 2 |
| 4-195 | F | H | CF₃ | EtNHCO | 2 |
| 4-196 | F | H | CF₃ | tert-BuOC(═O) | 2 |
| 4-197 | H | F | CF₃ | H | 2 |
| 4-198 | H | F | CF₃ | MeCO | 2 |
| 4-199 | H | F | CF₃ | EtCO | 2 |
| 4-200 | H | F | CF₃ | cyclo-PrCO | 2 |
| 4-201 | H | F | CF₃ | CH₃SCH₂CO | 2 |
| 4-202 | H | F | CF₃ | CF₃CH₂C0 | 2 |
| 4-203 | H | F | CF₃ | EtNHCO | 2 |
| 4-204 | H | F | CF₃ | tert-BuOC(═O) | 2 |
| 4-205 | OCF₃ | H | H | EtCO | 1 |
| 4-206 | SCF₃ | H | H | EtCO | 1 |
| 4-207 | SOCF₃ | H | H | EtCO | 1 |
| 4-208 | SO₂CF₃ | H | H | EtCO | 1 |

TABLE 5

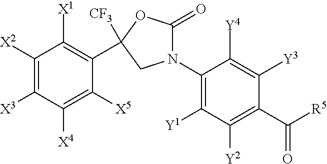

wherein X¹, X⁵, Y¹, Y² and Y⁴ represent hydrogen

| Cpd. No. | X² | X³ | X⁴ | Y³ | R⁵ |
|---|---|---|---|---|---|
| 24-1 | Cl | H | Cl | H | Me |
| 24-2 | Br | H | Br | H | Me |
| 24-3 | Cl | Cl | Cl | H | Me |
| 24-4 | CF₃ | H | H | H | Me |
| 24-5 | CF₃ | H | CF₃ | H | Me |
| 24-6 | Cl | Cl | CF₃ | H | Me |
| 24-7 | Cl | H | CF₃ | H | Me |
| 24-8 | F | H | CF₃ | H | Me |
| 24-9 | H | F | CF₃ | H | Me |
| 24-10 | OCF₃ | H | H | H | Me |
| 24-11 | SCF₃ | H | H | H | Me |
| 24-12 | SOCF₃ | H | H | H | Me |
| 24-13 | SO₂CF₃ | H | H | H | Me |
| 24-14 | Cl | H | Cl | CF₃ | H |
| 24-15 | Cl | H | Cl | CF₃ | MeO |
| 24-16 | Cl | H | Cl | CF₃ | EtO |
| 24-17 | Cl | H | Cl | CF₃ | tert-BuO |
| 24-18 | Cl | H | Cl | CF₃ | HO |
| 24-19 | Cl | H | Cl | Cl | H |
| 24-20 | Cl | H | Cl | Cl | MeO |
| 24-21 | Cl | H | Cl | Cl | EtO |
| 24-22 | Cl | H | Cl | Cl | tert-BuO |
| 24-23 | Cl | H | Cl | Cl | HO |
| 24-24 | Cl | H | Cl | Br | H |
| 24-25 | Cl | H | Cl | Br | Me0 |
| 24-26 | Cl | H | Cl | Br | EtO |
| 24-27 | Cl | H | Cl | Br | tert-BuO |
| 24-28 | Cl | H | Cl | Br | HO |
| 24-29 | Cl | H | Cl | Me | H |
| 24-30 | Cl | H | Cl | Me | MeO |
| 24-31 | Cl | H | Cl | Me | EtO |
| 24-32 | Cl | H | Cl | Me | tert-BuO |
| 24-33 | Br | H | Br | CF₃ | H |
| 24-34 | Br | H | Br | CF₃ | MeO |
| 24-35 | Br | H | Br | CF₃ | EtO |
| 24-36 | Br | H | Br | CF₃ | tert-BuO |
| 24-37 | Br | H | Br | CF₃ | HO |
| 24-38 | Br | H | Br | Cl | H |
| 24-39 | Br | H | Br | Cl | MeO |
| 24-40 | Br | H | Br | Cl | EtO |
| 24-41 | Br | H | Br | Cl | tert-BuO |
| 24-42 | Br | H | Br | Cl | HO |
| 24-43 | Br | H | Br | Br | H |
| 24-44 | Br | H | Br | Br | MeO |
| 24-45 | Br | H | Br | Br | EtO |
| 24-46 | Br | H | Br | Br | tert-BuO |
| 24-47 | Br | H | Br | Br | HO |
| 24-48 | Br | H | Br | Me | H |
| 24-49 | Br | H | Br | Me | MeO |
| 24-50 | Br | H | Br | Me | EtO |
| 24-51 | Br | H | Br | Me | tert-BuO |
| 24-52 | Br | H | Br | Me | HO |
| 24-53 | Cl | Cl | Cl | CF₃ | H |
| 24-54 | Cl | Cl | Cl | CF₃ | MeO |
| 24-55 | Cl | Cl | Cl | CF₃ | EtO |
| 24-56 | Cl | Cl | Cl | CF₃ | tert-BuO |
| 24-57 | Cl | Cl | Cl | CF₃ | HO |
| 24-58 | Cl | Cl | Cl | Cl | H |
| 24-59 | Cl | Cl | Cl | Cl | MeO |
| 24-60 | Cl | Cl | Cl | Cl | EtO |
| 24-61 | Cl | Cl | Cl | Cl | tert-BuO |
| 24-62 | Cl | Cl | Cl | Cl | HO |
| 24-63 | Cl | Cl | Cl | Br | H |
| 24-64 | Cl | Cl | Cl | Br | MeO |
| 24-65 | Cl | Cl | Cl | Br | EtO |
| 24-66 | Cl | Cl | Cl | Br | tert-BuO |
| 24-67 | Cl | Cl | Cl | Br | HO |
| 24-68 | Cl | Cl | Cl | Me | H |
| 24-69 | Cl | Cl | Cl | Me | MeO |
| 24-70 | Cl | Cl | Cl | Me | EtO |
| 24-71 | Cl | Cl | Cl | Me | tert-BuO |
| 24-72 | Cl | Cl | Cl | Me | HO |
| 24-73 | CF₃ | H | H | CF₃ | H |
| 24-74 | CF₃ | H | H | CF₃ | MeO |
| 24-75 | CF₃ | H | H | CF₃ | EtO |
| 24-76 | CF₃ | H | H | CF₃ | tert-BuO |
| 24-77 | CF₃ | H | H | CF₃ | HO |
| 24-78 | CF₃ | H | H | Cl | H |
| 24-79 | CF₃ | H | II | Cl | MeO |
| 24-80 | CF₃ | H | H | Cl | EtO |
| 24-81 | CF₃ | H | H | Cl | tert-BuO |
| 24-82 | CF₃ | H | H | Cl | HO |
| 24-83 | CF₃ | H | H | Br | H |
| 24-84 | CF₃ | H | H | Br | MeO |
| 24-85 | CF₃ | H | H | Br | EtO |
| 24-86 | CF₃ | H | H | Br | tert-BuO |
| 24-87 | CF₃ | H | H | Br | HO |
| 24-88 | CF₃ | H | H | Me | H |
| 24-89 | CF₃ | H | H | Me | MeO |
| 24-90 | CF₃ | H | H | Me | EtO |
| 24-91 | CF₃ | H | H | Me | tert-BuO |
| 24-92 | CF₃ | H | H | Me | HO |
| 24-93 | CF₃ | H | CF₃ | CF₃ | H |
| 24-94 | CF₃ | H | CF₃ | CF₃ | MeO |
| 24-95 | CF₃ | H | CF₃ | CF₃ | EtO |
| 24-96 | CF₃ | H | CF₃ | CF₃ | tert-BuO |
| 24-97 | CF₃ | H | CF₃ | CF₃ | HO |
| 24-98 | CF₃ | H | CF₃ | Cl | H |
| 24-99 | CF₃ | H | CF₃ | Cl | MeO |
| 24-100 | CF₃ | H | CF₃ | Cl | EtO |
| 24-101 | CF₃ | H | CF₃ | Cl | tert-BuO |
| 24-102 | CF₃ | H | CF₃ | Cl | HO |
| 24-103 | CF₃ | H | CF₃ | Br | H |
| 24-104 | CF₃ | H | CF₃ | Br | MeO |
| 24-105 | CF₃ | H | CF₃ | Br | EtO |
| 24-106 | CF₃ | H | CF₃ | Br | tert-BuO |
| 24-107 | CF₃ | H | CF₃ | Br | HO |
| 24-108 | CF₃ | H | CF₃ | Me | H |
| 24-109 | CF₃ | H | CF₃ | Me | MeO |
| 24-110 | CF₃ | H | CF₃ | Me | EtO |
| 24-111 | CF₃ | H | CF₃ | Me | tert-BuO |
| 24-112 | CF₃ | H | CF₃ | Me | HO |
| 24-113 | Cl | Cl | CF₃ | CF₃ | H |
| 24-114 | Cl | Cl | CF₃ | CF₃ | MeO |
| 24-115 | Cl | Cl | CF₃ | CF₃ | EtO |
| 24-116 | Cl | Cl | CF₃ | CF₃ | tert-BuO |
| 24-117 | Cl | Cl | CF₃ | CF₃ | HO |
| 24-118 | Cl | H | CF₃ | CF₃ | H |
| 24-119 | Cl | H | CF₃ | CF₃ | MeO |
| 24-120 | Cl | H | CF₃ | CF₃ | EtO |
| 24-121 | Cl | H | CF₃ | CF₃ | tert-BuO |
| 24-122 | Cl | H | CF₃ | CF₃ | HO |
| 24-123 | F | H | CF₃ | CF₃ | H |
| 24-124 | F | H | CF₃ | CF₃ | MeO |
| 24-125 | F | H | CF₃ | CF₃ | EtO |
| 24-126 | F | H | CF₃ | CF₃ | tert-BuO |
| 24-127 | F | H | CF₃ | CF₃ | HO |
| 24-128 | H | F | CF₃ | CF₃ | H |
| 24-129 | H | F | CF₃ | CF₃ | MeO |
| 24-130 | H | F | CF₃ | CF₃ | EtO |
| 24-131 | H | F | CF₃ | CF₃ | tert-BuO |
| 24-132 | H | F | CF₃ | CF₃ | HO |
| 24-133 | OCF₃ | H | H | CF₃ | H |
| 24-134 | OCF₃ | H | H | CF₃ | MeO |
| 24-135 | OCF₃ | H | H | CF₃ | EtO |
| 24-136 | OCF₃ | H | H | CF₃ | tert-BuO |
| 24-137 | OCF₃ | H | H | CF₃ | HO |
| 24-138 | SCF₃ | H | H | CF₃ | H |
| 24-139 | SCF₃ | H | H | CF₃ | MeO |
| 24-140 | SCF₃ | H | H | CF₃ | EtO |
| 24-141 | SCF₃ | H | H | CF₃ | tert -BuO |
| 24-142 | SCF₃ | H | H | CF₃ | HO |
| 24-143 | SOCF₃ | H | H | CF₃ | H |
| 24-144 | SOCF₃ | H | H | CF₃ | MeO |
| 24-145 | SOCF₃ | H | H | CF₃ | EtO |
| 24-146 | SOCF₃ | H | H | CF₃ | tert-BuO |
| 24-147 | SOCF₃ | H | H | CF₃ | HO |
| 24-148 | SO₂CF₃ | H | H | CF₃ | H |
| 24-149 | SO₂CF₃ | H | H | CF₃ | MeO |
| 24-150 | SO₂CF₃ | H | H | CF₃ | EtO |
| 24-151 | SO₂CF₃ | H | H | CF₃ | tert-BuO |
| 24-152 | SO₂CF₃ | H | H | CF₃ | HO |

TABLE 6

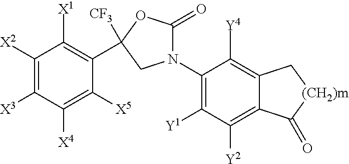

wherein X¹, X⁵, Y¹, Y² and Y⁴ represent hydrogen.

| Cpd. No. | X² | X³ | X⁴ | m |
|---|---|---|---|---|
| 25-1 | Cl | H | Cl | 1 |
| 25-2 | Br | H | Br | 1 |
| 25-3 | Cl | Cl | Cl | 1 |
| 25-4 | CF₃ | H | H | 1 |
| 25-5 | CF₃ | H | CF₃ | 1 |
| 25-6 | Cl | Cl | CF₃ | 1 |
| 25-7 | Cl | H | CF₃ | 1 |
| 25-8 | F | H | CF₃ | 1 |
| 25-9 | H | F | CF₃ | 1 |
| 25-10 | Cl | H | Cl | 2 |
| 25-11 | Br | H | Br | 2 |
| 25-12 | Cl | Cl | Cl | 2 |
| 25-13 | CF₃ | H | H | 2 |
| 25-14 | CF₃ | H | CF₃ | 2 |
| 25-15 | Cl | Cl | CF₃ | 2 |
| 25-16 | Cl | H | CF₃ | 2 |
| 25-17 | F | H | CF₃ | 2 |
| 25-18 | H | F | CF₃ | 2 |
| 25-19 | OCF₃ | H | H | 1 |
| 25-20 | SCF₃ | H | H | 1 |
| 25-21 | SOCF₃ | H | H | 1 |
| 25-22 | SO₂CF₃ | H | H | 1 |
| 25-23 | OCF₃ | H | H | 2 |
| 25-24 | SCF₃ | H | H | 2 |
| 25-25 | SOCF₃ | H | H | 2 |
| 25-26 | SO₂CF₃ | H | H | 2 |

TABLE 7

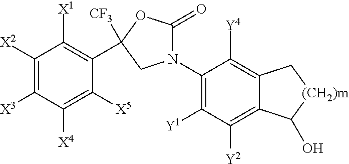

wherein X¹, X⁵, Y¹, Y² and Y⁴ represent hydrogen

| Cpd. No. | X² | X³ | X⁴ | m |
|---|---|---|---|---|
| 30-1 | Cl | H | Cl | 1 |
| 30-2 | Br | H | Br | 1 |
| 30-3 | Cl | Cl | Cl | 1 |
| 30-4 | CF₃ | H | H | 1 |
| 30-5 | CF₃ | H | CF₃ | 1 |
| 30-6 | Cl | Cl | CF₃ | 1 |
| 30-7 | Cl | H | CF₃ | 1 |
| 30-8 | F | H | CF₃ | 1 |
| 30-9 | H | F | CF₃ | 1 |
| 30-10 | Cl | H | Cl | 2 |
| 30-11 | Br | H | Br | 2 |
| 30-12 | Cl | Cl | Cl | 2 |
| 30-13 | CF₃ | H | H | 2 |
| 30-14 | CF₃ | H | CF₃ | 2 |
| 30-15 | Cl | Cl | CF₃ | 2 |
| 30-16 | Cl | H | CF₃ | 2 |
| 30-17 | F | H | CF₃ | 2 |
| 30-18 | H | F | CF₃ | 2 |
| 30-19 | OCF₃ | H | H | 1 |
| 30-20 | SCF₃ | H | H | 1 |
| 30-21 | SOCF₃ | H | H | 1 |
| 30-22 | SO₂CF₃ | H | H | 1 |
| 30-23 | OCF₃ | H | H | 2 |
| 30-24 | SCF₃ | H | H | 2 |
| 30-25 | SOCF₃ | H | H | 2 |
| 30-26 | SO₂CF₃ | H | H | 2 |

TABLE 8

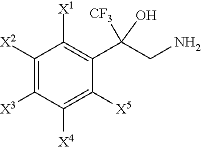

wherein X¹ and X⁵ represent hydrogen

| Cpd. No. | X² | X³ | X⁴ |
|---|---|---|---|
| 27-1 | Cl | H | Cl |
| 27-2 | Br | H | Br |
| 27-3 | Cl | Cl | Cl |
| 27-4 | CF₃ | H | H |
| 27-5 | CF₃ | H | CF₃ |
| 27-6 | Cl | Cl | CF₃ |
| 27-7 | Cl | H | CF₃ |
| 27-8 | F | H | CF₃ |
| 27-9 | H | F | CF₃ |
| 27-10 | OCF₃ | H | H |
| 27-11 | SCF₃ | H | H |
| 27-12 | SOCF₃ | H | H |
| 27-13 | SO₂CF₃ | H | H |

TABLE 9

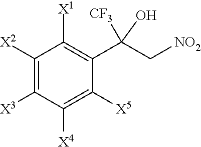

wherein X¹ and X⁵ represent hydrogen

| Cpd. No. | X² | X³ | X⁴ |
|---|---|---|---|
| 28-1 | Br | H | Br |
| 28-2 | Cl | Cl | Cl |
| 28-3 | CF₃ | H | CF₃ |
| 28-4 | Cl | Cl | CF₃ |
| 28-5 | Cl | H | CF₃ |
| 28-6 | F | H | CF₃ |
| 28-7 | H | F | CF₃ |
| 28-8 | OCF₃ | H | H |
| 28-9 | SCF₃ | H | H |
| 28-10 | SOCF₃ | H | H |
| 28-11 | SO₂CF₃ | H | H |

TABLE 10

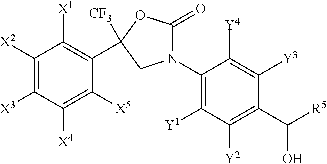

wherein X¹, X⁵, Y¹, Y² and Y⁴ represent hydrogen

| Cpd. No. | X² | X³ | X⁴ | Y³ | R⁵ |
|---|---|---|---|---|---|
| 29-1 | Cl | H | Cl | H | Mc |
| 29-2 | Br | H | Br | H | Me |
| 29-3 | Cl | Cl | Cl | H | Me |
| 29-4 | CF₃ | H | H | H | Me |
| 29-5 | CF₃ | H | CF₃ | H | Me |
| 29-6 | Cl | Cl | CF₃ | H | Me |
| 29-7 | Cl | H | CF₃ | H | Me |
| 29-8 | F | H | CF₃ | H | Me |
| 29-9 | H | F | CF₃ | H | Me |
| 29-10 | OCF₃ | H | H | H | Me |
| 29-11 | SCF₃ | H | H | H | Me |
| 29-12 | SOCF₃ | H | H | H | Me |
| 29-13 | SO₂CF₃ | H | H | H | Me |
| 29-14 | Cl | H | Cl | CF₃ | H |
| 29-15 | Cl | H | Cl | Cl | H |
| 29-16 | Cl | H | Cl | Br | H |
| 29-17 | Cl | H | Cl | Me | H |
| 29-18 | Br | H | Br | CF₃ | H |
| 29-19 | Br | H | Br | Cl | H |
| 29-20 | Br | H | Br | Br | H |
| 29-21 | Br | H | Br | Me | H |
| 29-22 | Cl | Cl | Cl | CF₃ | H |
| 29-23 | Cl | Cl | Cl | Cl | H |
| 29-24 | Cl | Cl | Cl | Br | H |
| 29-25 | Cl | Cl | Cl | Me | H |
| 29-26 | CF₃ | H | H | CF₃ | H |
| 29-27 | CF₃ | H | H | Cl | H |
| 29-28 | CF₃ | H | H | Br | H |
| 29-29 | CF₃ | H | H | Me | H |
| 29-30 | CF₃ | H | CF₃ | CF₃ | H |
| 29-31 | CF₃ | H | CF₃ | Cl | H |
| 29-32 | CF₃ | H | CF₃ | Br | H |
| 29-33 | CF₃ | H | CF₃ | Me | H |
| 29-34 | Cl | Cl | CF₃ | CF₃ | H |
| 29-35 | Cl | H | CF₃ | CF₃ | H |
| 29-36 | F | H | CF₃ | CF₃ | H |
| 29-37 | H | F | CF₃ | CF₃ | H |
| 29-38 | OCF₃ | H | H | CF₃ | H |
| 29-39 | SCF₃ | H | H | CF₃ | H |
| 29-40 | SOCF₃ | H | H | CF₃ | H |
| 29-41 | SO₂CF₃ | H | H | CF₃ | H |

TABLE 11

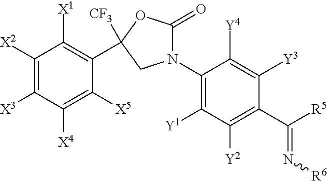

wherein X¹, X⁵, Y¹, Y² and Y⁴ represent hydrogen

| Cpd. No. | X² | X³ | X⁴ | Y³ | R⁵ | R⁶ |
|---|---|---|---|---|---|---|
| 32-1 | Cl | H | Cl | H | Me | HO |
| 32-2 | Br | H | Br | H | Me | HO |
| 32-3 | Cl | Cl | Cl | H | Me | HO |
| 32-4 | CF₃ | H | H | H | Me | HO |
| 32-5 | CF₃ | H | CF₃ | H | Me | HO |
| 32-6 | Cl | Cl | CF₃ | H | Me | HO |
| 32-7 | Cl | H | CF₃ | H | Me | HO |
| 32-8 | F | H | CF₃ | H | Me | HO |
| 32-9 | H | F | CF₃ | H | Me | HO |
| 32-10 | OCF₃ | H | H | H | Me | HO |
| 32-11 | SCF₃ | H | H | H | Me | HO |
| 32-12 | SOCF₃ | H | H | H | Me | HO |
| 32-13 | SO₂CF₃ | H | H | H | Me | HO |
| 32-14 | Cl | H | Cl | H | Me | MeO |
| 32-15 | Br | H | Br | H | Me | MeO |
| 32-16 | Cl | Cl | Cl | H | Me | MeO |
| 32-17 | CF₃ | H | H | H | Me | MeO |
| 32-18 | CF₃ | H | CF₃ | H | Me | MeO |
| 32-19 | Cl | Cl | CF₃ | H | Me | MeO |
| 32-20 | Cl | H | CF₃ | H | Me | MeO |
| 32-21 | F | H | CF₃ | H | Me | MeO |
| 32-22 | H | F | CF₃ | H | Me | MeO |
| 32-23 | OCF₃ | H | H | H | Me | MeO |
| 32-24 | SCF₃ | H | H | H | Me | MeO |
| 32-25 | SOCF₃ | H | H | H | Me | MeO |
| 32-26 | SO₂CF₃ | H | H | H | Me | MeO |
| 32-27 | Cl | H | Cl | CF₃ | H | HO |
| 32-28 | Br | H | Br | CF₃ | H | HO |
| 32-29 | Cl | Cl | Cl | CF₃ | H | HO |
| 32-30 | CF₃ | H | H | CF₃ | H | HO |
| 32-31 | CF₃ | H | CF₃ | CF₃ | H | HO |
| 32-32 | Cl | Cl | CF₃ | CF₃ | H | HO |
| 32-33 | Cl | H | CF₃ | CF₃ | H | HO |
| 32-34 | F | H | CF₃ | CF₃ | H | HO |
| 32-35 | H | F | CF₃ | CF₃ | H | HO |
| 32-36 | OCF₃ | H | H | CF₃ | H | HO |
| 32-37 | SCF₃ | H | H | CF₃ | H | HO |
| 32-38 | SOCF₃ | H | H | CF₃ | H | HO |
| 32-39 | SO₂CF₃ | H | H | CF₃ | H | HO |
| 32-40 | Cl | H | Cl | CF₃ | H | MeO |
| 32-41 | Br | H | Br | CF₃ | H | MeO |
| 32-42 | Cl | Cl | Cl | CF₃ | H | MeO |
| 32-43 | CF₃ | H | H | CF₃ | H | MeO |
| 32-44 | CF₃ | H | CF₃ | CF₃ | H | MeO |
| 32-45 | Cl | Cl | CF₃ | CF₃ | H | MeO |
| 32-46 | Cl | H | CF₃ | CF₃ | H | MeO |
| 32-47 | F | H | CF₃ | CF₃ | H | MeO |
| 32-48 | H | F | CF₃ | CF₃ | H | MeO |
| 32-49 | OCF₃ | H | H | CF₃ | H | MeO |
| 32-50 | SCF₃ | H | H | CF₃ | H | MeO |
| 32-51 | SOCF₃ | H | H | CF₃ | H | MeO |
| 32-52 | SO₂CF₃ | H | H | CF₃ | H | MeO |
| 32-53 | Cl | H | Cl | Cl | H | HO |
| 32-54 | Br | H | Br | Cl | H | HO |
| 32-55 | Cl | Cl | Cl | Cl | H | HO |
| 32-56 | CF₃ | H | H | Cl | H | HO |
| 32-57 | CF₃ | H | CF₃ | Cl | H | HO |
| 32-58 | Cl | Cl | CF₃ | Cl | H | HO |
| 32-59 | Cl | H | CF₃ | Cl | H | HO |
| 32-60 | F | H | CF₃ | Cl | H | HO |
| 32-61 | H | F | CF₃ | Cl | H | HO |
| 32-62 | OCF₃ | H | H | Cl | H | HO |
| 32-63 | SCF₃ | H | H | Cl | H | HO |
| 32-64 | SOCF₃ | H | H | Cl | H | HO |
| 32-65 | SO₂CF₃ | H | H | Cl | H | HO |
| 32-66 | Cl | H | Cl | Cl | H | MeO |
| 32-67 | Br | H | Br | Cl | H | MeO |
| 32-68 | Cl | Cl | Cl | Cl | H | MeO |
| 32-69 | CF₃ | H | H | Cl | H | MeO |
| 32-70 | CF₃ | H | CF₃ | Cl | H | MeO |
| 32-71 | Cl | Cl | CF₃ | Cl | H | MeO |
| 32-72 | Cl | H | CF₃ | Cl | H | MeO |
| 32-73 | F | H | CF₃ | Cl | H | MeO |
| 32-74 | H | F | CF₃ | Cl | H | MeO |
| 32-75 | OCF₃ | H | H | Cl | H | MeO |
| 32-76 | SCF₃ | H | H | Cl | H | MeO |
| 32-77 | SOCF₃ | H | H | Cl | H | MeO |
| 32-78 | SO₂CF₃ | H | H | Cl | H | MeO |
| 32-79 | Cl | H | Cl | Br | H | HO |
| 32-80 | Br | H | Br | Br | H | HO |
| 32-81 | Cl | Cl | Cl | Br | H | HO |
| 32-82 | CF₃ | H | H | Br | H | HO |
| 32-83 | CF₃ | H | CF₃ | Br | H | HO |
| 32-84 | Cl | Cl | CF₃ | Br | H | HO |
| 32-85 | Cl | H | CF₃ | Br | H | HO |
| 32-86 | F | H | CF₃ | Br | H | HO |
| 32-87 | H | F | CF₃ | Br | H | HO |
| 32-88 | OCF₃ | H | H | Br | H | HO |
| 32-89 | SCF₃ | H | H | Br | H | HO |
| 32-90 | SOCF₃ | H | H | Br | H | HO |
| 32-91 | SO₂CF₃ | H | H | Br | H | HO |
| 32-92 | Cl | H | Cl | Br | H | MeO |
| 32-93 | Br | H | Br | Br | H | MeO |
| 32-94 | Cl | Cl | Cl | Br | H | MeO |
| 32-95 | CF₃ | H | H | Br | H | MeO |
| 32-96 | CF₃ | H | CF₃ | Br | H | MeO |
| 32-97 | Cl | Cl | CF₃ | Br | H | MeO |
| 32-98 | Cl | H | CF₃ | Br | II | MeO |
| 32-99 | F | H | CF₃ | Br | H | MeO |
| 32-100 | H | F | CF₃ | Br | H | MeO |
| 32-101 | OCF₃ | H | H | Br | H | MeO |
| 32-102 | SCF₃ | H | H | Br | H | MeO |
| 32-103 | SOCF₃ | H | H | Br | H | MeO |
| 32-104 | SO₂CF₃ | H | H | Br | H | MeO |
| 32-105 | Cl | H | Cl | Me | H | HO |
| 32-106 | Br | H | Br | Me | H | HO |
| 32-107 | Cl | Cl | Cl | Me | H | HO |
| 32-108 | CF₃ | H | H | Me | H | HO |
| 32-109 | CF₃ | H | CF₃ | Me | H | HO |
| 32-110 | Cl | Cl | CF₃ | Me | H | HO |
| 32-111 | Cl | H | CF₃ | Me | H | HO |
| 32-112 | F | H | CF₃ | Me | H | HO |
| 32-113 | H | F | CF₃ | Me | H | HO |
| 32-114 | OCF₃ | H | H | Me | H | HO |
| 32-115 | SCF₃ | H | H | Me | H | HO |
| 32-116 | SOCF₃ | H | H | Me | H | HO |
| 32-117 | SO₂CF₃ | H | H | Me | H | HO |
| 32-118 | Cl | H | Cl | Me | H | MeO |
| 32-119 | Br | H | Br | Me | H | MeO |
| 32-120 | Cl | Cl | Cl | Me | H | MeO |
| 32-121 | CF₃ | H | H | Me | H | MeO |
| 32-122 | CF₃ | H | CF₃ | Me | H | MeO |
| 32-123 | Cl | Cl | CF₃ | Me | H | MeO |
| 32-124 | Cl | H | CF₃ | Me | H | MeO |
| 32-125 | F | H | CF₃ | Me | H | MeO |
| 32-126 | H | F | CF₃ | Me | H | MeO |
| 32-127 | OCF₃ | H | H | Me | H | MeO |
| 32-128 | SCF₃ | H | H | Me | H | MeO |
| 32-129 | SOCF₃ | H | H | Me | H | MeO |
| 32-130 | SO₂CF₃ | H | H | Me | H | MeO |

TABLE 12

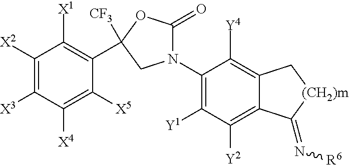

wherein X¹, X⁵, Y¹, Y² and Y⁴ represent hydrogen

| Cpd. No. | X² | X³ | X⁴ | m | R⁶ |
|---|---|---|---|---|---|
| 33-1 | Cl | H | Cl | 1 | HO |
| 33-2 | Br | H | Br | 1 | HO |
| 33-3 | Cl | Cl | Cl | 1 | HO |
| 33-4 | CF₃ | H | H | 1 | HO |
| 33-5 | CF₃ | H | CF₃ | 1 | HO |
| 33-6 | Cl | Cl | CF₃ | 1 | HO |
| 33-7 | Cl | H | CF₃ | 1 | HO |
| 33-8 | F | H | CF₃ | 1 | HO |
| 33-9 | H | F | CF₃ | 1 | HO |
| 33-10 | Cl | H | Cl | 1 | MeO |
| 33-11 | Br | H | Br | 1 | MeO |
| 33-12 | Cl | Cl | Cl | 1 | MeO |
| 33-13 | CF₃ | H | H | 1 | MeO |
| 33-14 | CF₃ | H | CF₃ | 1 | MeO |
| 33-15 | Cl | Cl | CF₃ | 1 | MeO |
| 33-16 | Cl | H | CF₃ | 1 | MeO |
| 33-17 | F | H | CF₃ | 1 | MeO |
| 33-18 | H | F | CF₃ | 1 | MeO |
| 33-19 | Cl | H | Cl | 2 | HO |
| 33-20 | Br | H | Br | 2 | HO |
| 33-21 | Cl | Cl | Cl | 2 | HO |
| 33-22 | CF₃ | H | H | 2 | HO |
| 33-23 | CF₃ | H | CF₃ | 2 | HO |
| 33-24 | Cl | Cl | CF₃ | 2 | HO |
| 33-25 | Cl | H | CF₃ | 2 | HO |
| 33-26 | F | H | CF₃ | 2 | HO |
| 33-27 | H | F | CF₃ | 2 | HO |
| 33-28 | Cl | H | Cl | 2 | MeO |
| 33-29 | Br | H | Br | 2 | MeO |
| 33-30 | Cl | Cl | Cl | 2 | MeO |
| 33-31 | CF₃ | H | H | 2 | MeO |
| 33-32 | CF₃ | H | CF₃ | 2 | MeO |
| 33-33 | Cl | Cl | CF₃ | 2 | MeO |
| 33-34 | Cl | H | CF₃ | 2 | MeO |
| 33-35 | F | H | CF₃ | 2 | MeO |
| 33-36 | H | F | CF₃ | 2 | MeO |
| 33-37 | OCF₃ | H | H | 1 | HO |
| 33-38 | SCF₃ | H | H | 1 | HO |
| 33-39 | SOCF₃ | H | H | 1 | HO |
| 33-40 | SO₂CF₃ | H | H | 1 | HO |
| 33-41 | OCF₃ | H | H | 2 | HO |
| 33-42 | SCF₃ | H | H | 2 | HO |
| 33-43 | SOCF₃ | H | H | 2 | HO |
| 33-44 | SO₂CF₃ | H | H | 2 | HO |
| 33-45 | OCF₃ | H | H | 1 | MeO |
| 33-46 | SCF₃ | H | H | 1 | MeO |
| 33-47 | SOCF₃ | H | H | 1 | MeO |
| 33-48 | SO₂CF₃ | H | H | 1 | MeO |

TABLE 13

| Cpd. No. | ¹H-NMR (CDCl₃) |
|---|---|
| 1-1 | δ: 4.31 (1H, d), 4.55 (2H, s), 4.64 (1H, d), 7.49 (3H, s), 7.75 (3H, s). |
| 1-2 | δ: 2.01 (3H, d), 4.29 (1H, d), 4.59-4.63 (3H, m), 5.79 (1H, br s), 7.50-7.50 (3H, m), |
| | 7.65-7.68 (2H, m), 7.81 (1H, s). |
| 1-3 | δ: 1.16 (3H, t), 2.18-2.27 (2H, m), 4.29 (1H, d), 4.59-4.63 (3H, m), 5.83 (1H, br s), |
| | 7.49-7.50 (3H, m), 7.62-7.71 (2H, m), 7.81 (1H, s). |
| 1-5 | δ: 0.78-0.83 (2H, m), 1.06-1.09 (2H, m), 1.25-1.27 (1H, m), 4.28 (1H, d), 4.62 (1H, d), |
| | 4.88-4.98 (3H, m), 7.49-7.51 (4H, m), 7.70-7.77 (2H, m). |
| 1-7 | δ: 3.05-3.15 (2H, m), 4.31 (1H, d), 4.62-4.64 (3H, m), 6.28 (1H, br s), 7.49-7.84 (6H, m). |
| 1-14 | δ: 1.45 (9H, s), 4.31 (1H, d), 4.47 (2H, d), 4.64 (1H, d), 4.99 (1H, br s), 7.50 (3H, s), |
| | 7.63-7.75 (3H, m). |
| 1-15 | δ: 4.01 (2H, s), 4.31 (1H, d), 4.65 (1H, d), 7.63-7.80 (5H, m). |
| 1-16 | δ: 2.01 (3H, s), 4.28 (1H, d), 4.58-4.63 (3H, m), 5.83 (1H, br s), 7.67-7.75 (5H, m). |
| 1-17 | δ: 1.16 (3H, t), 2.24 (2H, q), 4.29 (1H, d), 4.59-4.63 (3H, m), 5.86 (1H, br s), |
| | 7.66-7.76 (5H, m). |
| 1-21 | δ: 3.11 (2H, q), 4.29 (1H, d), 4.63-4.64 (3H, m), 6.15 (1H, br s), 7.60-7.85 (5H, m). |
| 1-28 | δ: 1.45 (9H, s), 4.28 (1H, d), 4.47 (2H, d), 4.63 (1H, d), 4.92 (1H, br s), 7.66-7.72 (5H, |
| | m). |
| 1-31 | δ: 1.16 (3H, t), 2.24 (2H, q), 4.25 (1H, d), 4.49 (2H, d), 4.59 (1H, d), 5.94 (1H, br s), |
| | 7.31-7.45 (2H, m), 7.62-7.64 (3H, m). |
| 1-42 | δ: 1.43 (9H, s), 4.22-4.38 (3H, m), 4.60 (1H, d), 4.99 (1H, br s), 7.31-7.61 (5H, m). |
| 1-59 | δ: 1.18 (3H, t), 2.26 (2H, q), 4.26 (1H, d), 4.43 (2H, d), 4.60 (1H, d), 5.78 (1H, br s), |
| | 7.32 (2H, d), 7.46 (2H, d), 7.62 (2H, s). |
| 1-70 | δ: 1.47 (9H, s), 4.26-4.29 (3H, m), 4.60 (1H, d), 4.86 (1H, br s), 7.31-7.62 (6H, m). |
| 1-72 | δ: 2.00 (3H, s), 4.36 (1H, d), 4.57 (2H, d), 4.70 (1H, d), 5.91 (1H, br s), 7.62-7.72 (3H, |
| | m), 7.77-7.85 (4H, m). |
| 1-73 | δ: 1.15 (3H, t), 2.24 (2H, q), 4.36 (1H, d), 4.58 (2H, d), 4.70 (1H, d), 5.93 (1H, br s), |
| | 7.63-7.68 (3H, m), 7.79-7.82 (4H, m). |
| 1-74 | δ: 0.91-0.93 (3H, m), 1.64-1.69 (2H, m), 2.17-2.19 (2H, m), 4.35 (1H, d), 4.59 (2H, d), |
| | 4.70 (1H, d), 5.79 (1H, br s), 7.62-7.83 (7H, m). |
| 1-75 | δ: 0.74-0.78 (2H, m), 0.96-1.01 (2H, m), 1.34-1.37 (1H, m), 4.35 (1H, d), 4.60 (2H, d), |
| | 4.70 (1H, d), 6.04 (1H, br s), 7.69-7.78 (7H, m). |
| 1-76 | δ: 0.16-0.21 (2H, m), 0.59-0.61 (2H, m), 0.90-0.93 (1H, m), 2.18 (3H, t), 4.35 (1H, d), |
| | 4.62 (2H, d), 4.70 (1H, d), 6.34 (1H, br s), 7.70-7.78 (7H, m). |
| 1-77 | δ: 3.10 (2H, q), 4.36 (1H, d), 4.63 (2H, d), 4.70 (1H, d), 6.14 (1H, br s), 7.60-7.87 (7H, |
| | m). |
| 1-84 | δ: 1.44 (9H, s), 4.38-4.46 (3H, m), 4.72 (1H, d), 5.04 (1H, br s), 7.58-7.86 (7H, m). |
| 1-127 | δ: 2.18 (2H, br s), 3.90 (2H, s), 4.32 (1H, d), 4.69 (1H, d), 7.55 (1H, d), 7.68 (2H, d), |
| | 7.95 (1H, s), 7.99 (2H, s). |
| 1-128 | δ: 2.04 (3H, s), 4.37 (1H, d), 4.57 (2H, d), 4.75 (1H, d), 5.94 (1H, d), 7.64-7.69 (2H, m), |
| | 7.82 (1H, d), 8.05 (3H, d). |
| 1-129 | δ: 1.16 (3H, t), 2.24 (2H, q), 4.37 (1H, d), 4.59 (2H, d), 4.75 (1H, d), 5.87 (1H, br t), |
| | 7.64-7.70 (2H, m), 7.82 (1H, d), 8.05 (3H, br s). |
| 1-131 | δ: 0.75-0.79 (2H, m), 0.96-0.99 (2H, m), 1.35-1.38 (1H, m), 4.36 (1H, d), 4.59 (2H, d), |
| | 4.75 (1H, d), 6.08 (1H, br t), 7.63-7.69 (2H, m), 7.81 (1H, d), 8.06 (3H, s). |
| 1-133 | δ: 3.10 (2H, q), 4.37 (1H, d), 4.63 (2H, d), 4.76 (1H, d), 6.18 (1H, s), 7.62-7.70 (2H, m), |
| | 7.86 (1H, d), 8.06 (3H, s). |
| 1-134 | δ: 2.08 (3H, s), 3.22 (2H, s), 4.35 (1H, d), 4.64 (2H, d), 4.75 (1H, d), 7.67-7.80 (3H, m), |
| | 8.05 (3H, s). |
| 1-136 | δ: 3.06 (3H, s), 3.91 (2H, s), 4.36 (1H, d), 4.68 (2H, d), 4.75 (1H, d), 6.75 (1H, s), |
| | 7.61-7.74 (2H, m), 7.83 (1H, s), 8.05 (3H, s). |
| 1-137 | δ: 2.49 (2H, t), 3.36 (3H, s), 3.62 (2H, t), 4.36 (1H, d), 4.60 (2H, d), 4.75 (1H, d), |
| | 6.76 (1H, br t), 7.63-7.73 (2H, m), 7.81 (1H, d), 8.05 (3H, s). |
| 1-140 | δ: 1.44 (9H, s), 4.39-4.46 (3H, m), 4.77 (1H, d), 6.80-6.80 (1H, m), 7.59-7.73 (3H, m), |
| | 8.04 (1H, s), 8.07 (2H, s). |
| 1-141 | δ: 1.55 (2H, br s), 3.91 (2H, br s), 4.34 (1H, d), 4.73 (1H, d), 7.40-7.45 (2H, m), |
| | 7.59 (1H, br s), 8.05 (3H, br s). |
| 1-142 | δ: 2.03 (3H, s), 4.34 (1H, d), 4.47 (2H, d), 4.71 (1H, d), 6.10 (1H, s), 7.27-7.42 (2H, m), |
| | 7.64 (1H, d), 8.05 (3H, br s). |
| 1-143 | δ: 1.20 (3H, t), 2.26 (2H, q), 4.33 (1H, d), 4.45 (2H, d), 4.73 (1H, d), 5.74 (1H, s), |
| | 7.33-7.35 (1H, m), 7.49-7.50 (2H, m), 8.05 (3H, s). |
| 1-145 | δ: 0.74-0.77 (2H, m), 0.96-0.99 (2H, m), 1.35-1.40 (1H, m), 4.32 (1H, d), 4.51 (2H, d, J = 6.0 Hz), |
| | 4.70 (1H, d), 6.12 (1H, br s), 7.36-7.43 (2H, m), 7.66 (1H, d), 8.05 (3H, s). |
| 1-147 | δ: 1.26 (3H, t), 3.10 (2H, q), 4.32 (1H, d), 4.54 (2H, d), 4.71 (1H, d), 6.23 (1H, s), |
| | 7.37-7.43 (2H, m), 7.70 (1H, d), 8.04 (3H, s). |
| 1-148 | δ: 2.10 (3H, s), 3.21 (2H, s), 4.36 (1H, d), 4.52 (2H, d), 4.73 (1H, d), 7.39-7.42 (3H, m), |
| | 7.66 (1H, d), 8.05 (3H, s). |
| 1-149 | δ: 2.64 (3H, s), 3.25 (1H, d), 3.71 (1H, d), 4.31 (1H, d), 4.57-4.59 (2H, m), 4.71 (1H, d), |
| | 7.35-7.65 (3H, m), 8.04 (3H, br s). |
| 1-150 | δ: 3.05 (3H, s), 3.91 (2H, s), 4.34 (1H, d), 4.53 (2H, d), 4.71 (1H, d), 7.04 (1H, s), |
| | 7.40-7.43 (2H, m), 7.62-7.63 (1H, m), 8.05 (3H, s). |
| 1-151 | δ: 2.49 (2H, t), 3.37 (3H, s), 3.63 (2H, t), 4.31 (1H, d), 4.50 (2H, d), 4.70 (1H, d), |
| | 6.78 (1H, s), 7.37-7.44 (2H, m), 7.66 (1H, d), 8.04 (3H, s). |
| 1-154 | δ: 1.45 (9H, s), 4.30-4.33 (3H, m), 4.72 (1H, d), 7.32-7.35 (1H, m), 7.48-7.51 (2H, m), |
| | 8.04 (3H, s). |
| 1-155 | δ: 2.10 (2H, br s), 2.36 (3H, s), 3.85 (2H, d), 4.32 (1H, d), 4.72 (1H, d), 7.34-7.37 (3H, |
| | m), 8.03 (1H, s), 8.05 (2H, s). |
| 1-156 | δ: 2.03 (3H, s), 2.31 (3H, s), 4.33-4.39 (3H, m), 4.71 (1H, d), 5.78 (1H, s), |
| | 7.23-7.34 (3H, m), 8.03 (1H, s), 8.06 (2H, s). |
| 1-157 | δ: 1.18 (3H, t), 2.25 (3H, q), 2.36 (3H, s), 4.32 (1H, d), 4.41 (2H, d), 4.71 (1H, d), |
| | 5.55 (1H, s), 7.25-7.35 (3H, m), 8.03 (1H, s), 8.05 (2H, s). |
| 1-159 | δ: 0.74-0.79 (2H, m), 0.91-0.96 (2H, m), 1.28-1.37 (1H, m), 2.37 (3H, s), 4.32 (1H, d), |
| | 4.43 (2H, d), 4.71 (1H, d), 5.72 (1H, s), 7.28-7.33 (3H, m), 8.03 (1H, s), 8.05 (2H, s). |
| 1-161 | δ: 2.29 (3H, s), 3.04-3.09 (3H, m), 4.33 (1H, d), 4.41 (2H, t), 4.71 (1H, d), 6.19 (1H, s), |
| | 7.21-7.33 (3H, m), 8.03 (1H, s), 8.05 (2H, s). |
| 1-162 | δ: 2.11 (3H, s), 2.37 (3H, s), 3.23 (2H, s), 4.36 (1H, d), 4.45 (2H, d), 4.73 (1H, d), |
| | 7.03 (1H, s), 7.29-7.36 (3H, m), 8.03 (1H, s), 8.06 (2H, s). |
| 1-163 | δ: 2.39 (3H, s), 2.65 (3H, s), 3.25 (1H, d), 3.70 (1H, d), 4.31 (1H, d), 4.47-4.49 (2H, m), |
| | 4.71 (1H, d), 7.34-7.36 (3H, m), 8.03 (1H, s), 8.05 (2H, s). |
| 1-164 | δ: 2.37 (3H, s), 3.06 (3H, s), 3.90 (2H, s), 4.32 (1H, d), 4.48 (2H, d), 4.71 (1H, d), |
| | 6.58 (1H, s), 7.38-7.54 (3H, m), 8.05 (3H, br s). |
| 1-165 | δ: 2.33 (3H, s), 2.50 (2H, t), 3.36 (3H, s), 3.63 (2H, t), 4.32 (1H, d), 4.41 (2H, d), |
| | 4.72 (1H, d), 6.42 (1H, s), 7.26-7.35 (3H, m), 8.03 (1H, s), 8.06 (2H, s). |
| 1-345-a | δ: 0.72-0.73 (2H, m), 0.92-0.98 (2H, m), 1.31-1.35 (1H, m), 1.49 (3H, d), 4.26 (1H, d), |
| | 4.59 (1H, d), 5.10-5.12 (1H, m), 5.81 (1H, s), 7.36 (2H, d), 7.46-7.48 (5H, m). |
| 1-369 | δ: 1.29 (3H, d), 4.00-4.11 (1H, m), 4.20 (1H, d), 4.54 (1H, d), 7.30-7.40 (3H, m), |
| | 7.45 (1H, s), 7.55 (2H, s). |
| 1-370 | δ: 1.44 (3H, d), 1.97 (3H, s), 4.27 (1H, d), 4.57-4.64 (1H, m), 5.04-5.14 (1H, m), |
| | 5.89 (1H, d), 7.34 (2H, d), 7.44 (2H, d), 7.60 (2H, s). |
| 1-371 | δ: 1.13-1.16 (3H, m), 2.19-2.22 (2H, m), 4.26 (1H, d), 4.59 (1H, d), 5.09-5.11 (1H, m), |
| | 5.73 (1H, br s), 7.34 (2H, d), 7.45 (2H, d), 7.61 (2H, s). |
| 1-371-b | δ: 1.13 (3H, t), 1.44 (3H, d), 2.20 (2H, q), 4.30 (1H, d), 4.60 (1H, d), 5.04-5.08 (1H, m), |
| | 6.18 (1H, d), 7.29-7.32 (2H, m), 7.40-7.44 (2H, m), 7.62 (2H, s). |
| 1-373 | δ: 0.72-0.75 (2H, m), 0.95-0.96 (2H, m), 1.26-1.35 (1H, m), 1.50 (3H, d), 4.25 (1H, d), |
| | 4.59 (1H, d), 5.10-5.12 (1H, m), 5.83 (1H, d), 7.35-7.38 (2H, m), 7.45-7.48 (2H, m), |
| | 7.61 (2H, s). |
| 1-373-a | δ: 0.73-0.78 (2H, m), 0.93-0.95 (2H, m), 1.30-1.38 (1H, m), 1.48 (3H, d), 4.26 (1H, d), |
| | 4.59 (1H, d), 5.06-5.15 (1H, m), 5.89 (1H, d), 7.36 (2H, d), 7.46 (2H, d), 7.61 (2H, s). |
| 1-382 | δ: 1.32 (12H, br s), 4.27 (1H, d), 4.60 (1H, d), 4.72 (1H, br s), 4.96 (1H, br s), |
| | 7.15-7.18 (2H, m), 7.41-7.43 (2H, m), 7.61 (2H, s). |
| 1-13 | δ: 1.12 (3H, t), 3.19-3.21 (2H, m), 4.29 (1H, d), 4.41 (1H, br s), 4.51-4.53 (2H, m), |
| | 4.62 (1H, d), 4.81 (1H, br s), 7.49 (3H, s), 7.67 (2H, s), 7.77 (1H, s). |
| 1-235 | δ: 3.10 (2H, q), 4.24 (1H, d), 4.55-4.58 (3H, m), 6.24 (1H, s), 7.43 (2H, s), 7.61 (2H, s), |
| | 7.83 (1H, s). |
| 1-18 | δ: 0.92-0.94 (3H, m), 1.65-1.68 (2H, m), 2.17-2.20 (2H, m), 4.28 (1H, d), 4.60-4.63 (3H, |
| | m), 5.79 (1H, br s), 7.62-7.80 (5H, m). |
| 1-19 | δ: 0.74-0.78 (2H, m), 0.96-0.99 (2H, m), 1.37-1.40 (1H, m), 4.33 (1H, d), 4.59-4.64 (3H, |
| | m), 6.19 (1H, br s), 7.64-7.74 (5H, m). |
| 1-20 | δ: 0.16-0.20 (2H, m), 0.59-0.62 (2H, m), 0.91-0.93 (1H, m), 2.18 (2H, d), 4.29 (1H, d), |
| | 4.62-4.64 (3H, m), 6.34 (1H, br s), 7.62-7.82 (5H, m). |
| 1-22 | δ: 2.04 (3H, s), 3.23 (2H, s), 4.29 (1H, d), 4.62-4.65 (3H, m), 7.66-7.78 (5H, m). |
| 1-25 | δ: 2.49 (2H, t), 3.35 (3H, s), 3.62 (2H, t), 4.28 (1H, d), 4.60-4.63 (3H, m), 6.74 (1H, br |
| | s), 7.53-7.79 (5H, m). |
| 1-27 | δ: 1.11-1.14 (3H, m), 3.19-3.22 (2H, m), 4.28 (1H, d), 4.36 (1H, br s), 4.53 (2H, d), |
| | 4.62 (1H, d), 4.76 (1H, br s), 7.62-7.77 (5H, m). |
| 2-1 | δ: 3.86 (1H, d), 4.24 (1H, d), 7.40 (2H, s), 7.46 (1H, s). |
| 2-3 | δ: 3.96 (1H, d), 4.34 (1H, d), 7.06 (1H, s), 7.62-7.73 (4H, m). |
| 2-2 | δ: 3.86 (1H, d), 4.25 (1H, d), 5.63 (1H, br s), 7.53 (2H, s). |
| 2-4 | δ: 3.93 (1H, d), 4.37 (1H, d), 5.38 (1H, br s), 7.97 (2H, s), 8.01 (1H, s). |
| 3-1 | δ: 4.38 (1H, d), 4.71 (1H, d), 7.50-7.51 (3H, m), 7.89-7.96 (3H, m). |
| 3-2 | δ: 4.34 (1H, d), 4.69 (1H, d), 7.62 (2H, s), 7.92-7.96 (3H, m). |
| 3-3 | δ: 4.29 (1H, d), 4.64 (1H, d), 7.62-7.74 (5H, m). |
| 3-5 | δ: 4.30 (1H, d), 4.65 (1H, d), 7.61 (2H, s), 7.68-7.72 (4H, m). |
| 3-6 | δ: 4.40 (1H, d), 4.75 (1H, d), 7.77-7.90 (7H, m). |
| 3-10 | δ: 4.40 (1H, d), 4.80 (1H, d), 7.90-7.97 (3H, m), 8.06 (3H, s). |
| 3-11 | δ: 4.37 (1H, d), 4.76 (1H, d), 7.65-7.77 (3H, m), 8.05 (3H, s). |
| 3-12 | δ: 2.59 (3H, s), 4.35 (1H, d), 4.75 (1H, d), 7.55-7.61 (3H, m), 8.05 (3H, s). |
| 3-19 | δ: 4.34 (1H, d), 4.66 (1H, d), 7.62 (2H, s), 7.68-7.69 (2H, m), 7.92 (1H, s). |
| 4-29 | δ: 2.03-2.12 (1H, m), 2.39-2.52 (1H, m), 2.86-3.05 (2H, m), 4.24-4.27 (1H, m), |
| | 4.58-4.61 (1H, m), 4.95-4.97 (1H, m), 7.25-7.40 (3H, m), 7.62 (2H, s). |
| 4-31 | δ: 1.21 (3H, t), 1.78-1.87 (1H, m), 2.25 (2H, q), 2.54-2.66 (1H, m), 2.81-3.04 (2H, m), |
| | 4.26-4.29 (1H, m), 4.59-4.62 (1H, m), 5.43-5.49 (1H, m), 5.68-5.71 (1H, m), |
| | 7.26-7.31 (2H, m), 7.45 (1H, d), 7.63 (2H, s). |
| 4-33 | δ: 0.77-0.81 (2H, m), 0.99-1.02 (2H, m), 1.35-1.37 (1H, m), 1.81-1.87 (1H, m), |
| | 2.55-2.65 (1H, m), 2.80-3.03 (2H, m), 4.26-4.29 (1H, m), 4.59-4.62 (1H, m), 5.46-5.48 (1H, m), |
| | 5.91 (1H, d), 7.21-7.33 (2H, m), 7.46 (1H, d), 7.60 (2H, s). |
| 4-34 | δ: 0.00-0.02 (2H, m), 0.36-0.43 (2H, m), 0.75-0.79 (1H, m), 1.60-1.66 (1H, m), 2.03 (2H, |
| | d), 2.43-2.47 (1H, m), 2.64-2.85 (2H, m), 4.06-4.10 (1H, m), 4.40-4.43 (1H, m), |
| | 5.30-5.33 (1H, m), 5.89 (1H, d), 7.06-7.15 (2H, m), 7.27 (1H, d), 7.44 |
| 4-35 | δ: 1.81-1.87 (1H, m), 2.55-2.65 (1H, m), 2.86-2.98 (1H, m), 3.07-3.13 (2H, m), |
| | 4.27-4.30 (1H, m), 4.58-4.61 (1H, m), 5.43-5.45 (1H, m), 6.26-6.28 (1H, m), 7.20-7.25 (2H, m), |
| | 7.48 (1H, d), 7.60 (2H, s). |
| 4-42 | δ: 1.49 (9H, s), 1.77-1.84 (1H, m), 2.59-2.60 (1H, m), 2.79-3.01 (2H, m), 4.25-4.28 (1H, |
| | m), 4.59-4.62 (1H, m), 4.70-4.72 (1H, m), 5.16 (1H, br s), 7.30-7.45 (3H, m), 7.62 (2H, |
| | s). |
| 4-3 | δ: 1.20 (3H, t), 1.82-1.84 (1H, m), 2.25 (2H, q), 2.61-2.64 (1H, m), 2.85-2.99 (2H, m), |
| | 4.27 (1H, d), 4.60 (1H, d), 5.49-5.54 (2H, m), 7.27-7.31 (3H, m), 7.45 (1H, s), 7.48 (2H, |
| | s). |
| 24-3 | δ: 2.62 (3H, s), 4.33 (1H, d), 4.68 (1H, d), 7.63-7.65 (4H, m), 8.00-8.02 (2H, m). |
| 24-55 | δ: 1.39 (3H, t), 4.34-4.39 (3H, m), 4.67 (1H, d), 7.63 (2H, s), 7.85-7.88 (3H, m). |
| 24-56 | δ: 1.58 (9H, s), 4.33 (1H, d), 4.66 (1H, d), 7.63 (2H, s), 7.80-7.83 (3H, m). |
| 24-57 | δ: 4.36 (1H, d), 4.70 (1H, d), 7.64 (2H, s), 7.91-8.12 (3H, m). |
| 27-1 | δ: 2.92 (1H, d), 3.54 (1H, d), 5.61 (1H, br s), 7.36 (1H, t), 7.47 (2H, d). |
| 27-3 | δ: 2.91 (1H, d), 3.54 (1H, d), 7.60 (2H, s). |
| 27-4 | δ: 1.42 (2H, br s), 2.98 (1H, d), 3.62 (1H, d), 7.45-7.96 (4H, m). |
| 27-5 | δ: 2.93 (1H, d, J = 13.2 Hz), 3.70 (1H, d, J = 13.4 Hz), 5.86 (1H, br s), 7.89 (1H, s), |
| | 8.05 (2H, s). |
| 28-2 | δ: 4.83 (1H, br s), 5.00 (2H, s), 7.63 (2H, s). |
| 28-3 | δ: 4.95 (1H, s), 5.10 (2H, s), 8.00 (1H, s), 8.08 (2H, s). |

The following biological examples demonstrate that the oxazolidinone derivatives of the present invention have an excellent pesticidal activity against agricultural pests and veterinary pests.

### BIOLOGICAL TEST EXAMPLES

Unless described otherwise, the test solutions were prepared as follows:

The active compound, 1 part by weight of the active compound was admixed with the solvent in the above described amount which contains the emulsifying agent in the above described amount. The mixture was then diluted with water to specific concentration.

In order to prepare the test solution containing the active compound, 1 part by weight of the active compound was admixed with 3 parts by weight of the solvent dimethyl formamide which contains 1 part by weight of the emulsifying agent polyoxyethylene alkylphenyl ether. This mixture is then diluted with water to the specific concentration.

### Biological Test Example 1

### Test Against Tobacco Cutworm (Spodoptera litura) Larvae

Solvent: dimethyl formamide, 3 parts by weight; emulsifying agent: polyoxyethylene alkylphenyl ether, 1 part by weight

Leaves of sweet potato were immersed in the test solution at the appropriate concentration, and the leaves were dried in air. The leaves were then placed in a petri dish having a diameter of 9 cm, and ten Spodoptera litura at third instar larvae were released therein. The petri dishes were placed in a temperature-controlled chamber at 25° C. After 2 days and 4 days more sweet potato leaves were added. After 7 days, the number of dead larvae was counted to calculate the insecticidal activity. An insecticidal activity of 100% means that all larvae were killed, whereas an insecticidal activity of 0% means that no larva was killed. In the current test, the results of two petri dishes for each treatment were averaged.

In the biological test example 1, the compounds Nos. 1-2, 1-3, 1-5, 1-7, 1-13, 1-14, 1-15, 1-16, 1-17, 1-18, 1-19, 1-20, 1-21, 1-22, 1-25, 1-28, 1-31, 1-59, 1-72, 1-73, 1-74, 1-75, 1-76, 1-77, 1-128, 1-129, 1-131, 1-133, 1-134, 1-136, 1-142, 1-143, 1-145, 1-147, 1-149, 1-151, 1-156, 1-157, 1-159, 1-161, 1-165, 1-345-a, 1-370, 1-371, 1-371-b, 1-373, 1-373-a, 3-1, 3-19, 4-3, 4-31, 4-33, 4-34, 4-35, showed an insecticidal activity of 100% at an active compound concentration of 100 ppm.

### Biological Test Example 2

### Test Against Two-Spotted Spider Mite (Tetranychus urticae)

Solvent: dimethyl formamide, 3 parts by weight; emulsifying agent: polyoxyethylene alkylphenyl ether, 1 part by weight

50 to 100 adult mites of Tetranychus urticae were inoculated to leaves of kidney bean at two-leaf stage planted in a pot of 6 cm in diameter. After one day, test solution at the appropriate concentration was sprayed thereon in a sufficient amount using a spray gun. After the spraying, the plant pot was placed inside a greenhouse, and after 7 days, the acaricidal activity was calculated. An acaricidal activity of 100% means that all mites were killed, whereas an acaricidal activity of 0% means that no mite was killed.

In the biological test example 2, the compound Nos. 1-3, 1-7, 1-16, 1-17, 1-21, 1-25, 1-31, 1-75, 1-76, 1-129, 1-133, 1-134, 1-136, 1-137, 1-145, 1-147, 1-345-a, 1-371, 1-373, 1-373-a, 4-31, 4-33, 4-34 and 4-35 showed an acaricidal activity of 100% at an active compound concentration of 100 ppm.

### Biological Test Example 3

### Test Against Cucurbit Leaf Beetle (Aulacophora femoralis)

Solvent: dimethyl formamide, 3 parts by weight; emulsifying agent: polyoxyethylene alkylphenyl ether, 1 part by weight

Leaves of cucumber were immersed in the test solution at the appropriate concentration, and the leaves were dried in air. The leaves were then put in a plastic cup containing sterilized black soil and five Aulacophora femoralis at second instar larvae were released in the cup. The cups were placed in a temperature-controlled chamber at 25° C. After 7 days, the number of dead larvae was counted, and thus the insecticidal activity was calculated. An insecticidal activity of 100% means that all larvae were killed, whereas an insecticidal activity of 0% means that no larva was killed.

In the biological test example 3, the compounds Nos. 1-2, 1-3, 1-5, 1-7, 1-14, 1-16, 1-17, 1-19, 1-21, 1-22, 1-28, 1-31, 1-59, 1-72, 1-73, 1-74, 1-75, 1-76, 1-77, 1-128, 1-129, 1-131, 1-133, 1-134, 1-136, 1-137, 1-142, 1-143, 1-145, 1-147, 1-148, 1-149, 1-157, 1-159, 1-161, 1-165, 1-370, 1-373-a, 4-31, 4-33, 4-34 and 4-35 showed an insecticidal activity of 100% at an active compound concentration of 100 ppm.

### Biological Test Example 4

### Boophilus microplus (Dip)

Solvent: dimethylsulfoxid

To produce a suitable preparation of active compound, 10 mg of active compound are dissolved in 0.5 ml solvent, and the concentrate is diluted with water to the desired concentration. Eight to ten adult engorged female Boophilus microplus ticks are placed in perforated plastic beakers and immersed in aqueous compound solution for one minute. Ticks are transferred to a filter paper in a plastic tray. Egg deposition of fertile eggs is monitored after. After 7 days mortality in % is determined. 100% means that all the ticks have been killed; 0% means that none of the ticks have been killed.

In this test for example, the following compounds from the preparation examples showed good activity of 80% at an application rate of 100 ppm: 1-2, 1-16

In this test for example, the following compounds from the preparation examples showed good activity of 100% at an application rate of 100 ppm: 1-17

### Biological Test Example 5

### Boophilus microplus-Test (BOOPMI Injection)

Solvent: dimethyl sulfoxide

To produce a suitable preparation Of active compound, 10 mg of active compound are dissolved in 0.5 ml solvent, and the concentrate is diluted with solvent to the desired concentration. Five adult engorged female ticks (Boophilus microplus) are injected with 1 n1 compound solution into the abdomen. Ticks are transferred into replica plates and incubated in a climate chamber for a period of time. Egg deposition of fertile eggs is monitored.

After 7 days, mortality in % is determined. 100% means that all eggs are infertile; 0% means that all eggs are fertile.

In this test for example, the following compounds from the preparation examples showed good activity of 100% at application rate of 20 μg/animal: 1-2, 1-3, 1-7, 1-16, 1-17, 1-21, 1-31, 1-59, 1-73, 1-75, 1-77, 1-142, 1-145, 1-147, 1-159, 1-161, 1-163, 4-31, 1-373-a.

### Biological Test Example 6

### Lucilia cuprina-Test (LUCICU 48 h)

Species: Lucilia cuprina 1^{st} instar larvae (age 24 hrs)

Solvent: dimethyl sulfoxide

10 mg active compound are dissolve in 0.5 ml Dimethylsulfoxid. Serial dilutions are made to obtain the desired rates. Approximately 20 Lucilia cuprina 1^{st} instar larvae are transferred into a test tube containing 1 cm³ of minced horse meat and 0.5 ml aqueous dilution of test compound. After 2 days percentage of larval mortality are recorded. 100% efficacy=all larvae are killed, % efficacy=normally developed larvae after 48 hrs.

In this test for example, the following compounds from the preparation examples showed good activity of 100% at application rate of 100 ppm: 1-2, 1-3, 1-7, 1-16, 1-17, 1-21, 1-31, 1-59, 1-73, 1-75, 1-77, 1-142, 1-145, 1-147, 1-159, 1-161, 4-31, 1-373-a.

### Biological Test Example 7

### Ctenocephalides felis-Test (CTECFE)

Solvent: dimethyl sulfoxide

To produce a suitable preparation of active compound, 10 mg of active compound are dissolved in 0.5 ml solvent, and the concentrate is diluted with cattle blood to the desired concentration. Approximately 20 adult unfed (Ctenocephalides felis) are placed in flea chambers. The blood chamber, sealed with parafilm on the bottom, are filled with cattle blood supplied with compound solution and placed on top of the flea chamber, so that the fleas are able to suck the blood. The blood chamber is heated to 37° C. whereas the flea chamber is kept at room temperature. After 2 days mortality in % is determined. 100% means that all the fleas have been killed; 0% means that none of the fleas have been killed.

In this test for example, the following compounds from the preparation examples showed good activity of 80% at application rate of 100 ppm: 1-161, 1-163.

In this test for example, the following compounds from the preparation examples showed good activity of 95% at application rate of 100 ppm: 1-2, 1-3, 1-7, 1-59, 1-145, 1-147, 1-159.

In this test for example, the following compounds from the preparation examples showed good activity of 100% at application rate of 100 ppm: 1-16, 1-17, 1-21, 1-31, 1-73, 1-75, 1-77, 1-142, 4-31, 1-373-a.

### Biological Test Example 8

### Musca domestica-Test

Solvent: dimethyl sulfoxide

To produce a suitable preparation of active compound, 10 mg of active compound are dissolved in 0.5 ml solvent, and the concentrate is diluted with water to the desired concentration. Prior to the assay, a piece of kitchen sponge is soaked with a mixture of sugar and compound solution and placed into a container. 10 adults (Musca domestica) are placed into the container and closed with a perforated lid. After 2 days mortality in % is determined. 100% means that all the flies have been killed; 0% means that none of the flies have been killed.

In this test for example, the following compounds from the preparation examples showed good activity of 80% at application rate of 100 ppm: 1-73, 1-145.

In this test for example, the following compounds from the preparation examples showed good activity of 90% at application rate of 100 ppm: 1-16, 1-17, 1-159.

In this test for example, the following compounds from the preparation examples showed good activity of 100% at application rate of 100 ppm: 1-2, 1-3, 1-7, 1-21, 1-75, 1-77, 1-147, 1-161, 4-31, 1-373-a.

### PREPARATION/FORMULATION EXAMPLES

### Preparation Example 1

### Granule Formulation

To a mixture containing the compound of the present invention (Compound No. 1-3; 10 parts by weight), bentonite (montmorilonite; 30 parts by weight), talc (58 parts by weight) and lignin sulfonate (2 parts by weight), water (25 parts by weight) is added and the resulting mixture is kneaded well. By using an extrusive granulator, granules of 10 to 40 mesh are formed and a granule formulation is obtained after drying at 40 to 50° C.

### Preparation Example 2

### Granule Formulation

Clay mineral having a size distribution in the range of 0.2 to 2 mm (95 parts by weight) is added to a rotary mixer. By spraying the compound of the present invention (Compound No. 1-3; 5 parts by weight) together with a liquid diluent under rotation, the clay is moistened followed by drying at 40 to 50° C. to obtain a granule formulation.

### Preparation Example 3

### Emulsion

By mixing the compound of the present invention (Compound No. 1-3; 30 parts by weight), xylene (55 parts by weight), polyoxyethylene alkylphenyl ether (8 parts by weight) and calcium alkylbenzene sulfonate (7 parts by weight) with stirring, an emulsion is obtained.

### Preparation Example 4

### Wettable Powder

By mixing and pulverizing the compound of the present invention (Compound No. 1-3; 15 parts by weight), a mixture (80 parts by weight) containing white carbon (fine powders of hydrous non-crystalline silicon oxide) and powder clay (1:5 mixture), sodium alkylbenzene sulfonate (2 parts by weight) and a condensate of sodium alkylnaphthalene sulfonate formalin (3 parts by weight), wettable powder is obtained.

### Preparation Example 5

### Wettable Granule

The compound of the present invention (Compound No. 1-3; 20 parts by weight), lignin sodium sulfonate (30 parts by weight), bentonite (15 parts by weight) and calcined diatomite powder (35 parts by weight) are thoroughly mixed. After adding water thereto, the mixture is extruded through 0.3 mm screen followed by drying to obtain wettable granule.

As shown in the above examples, the novel oxazolidinone derivatives of the present invention have an excellent pesticidal activity as pesticidal agents.

1. Azolidine derivatives of formula (I) 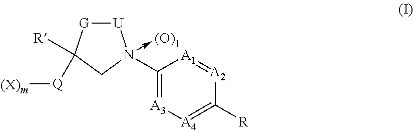
wherein
R′ represents C₁₋₁₂ alkyl or C₁₋₁₂ haloalkyl which may be substituted;
l represents 0 or 1;
G represents CH, CH₂, N, O, S, C═O, C═S, or NR¹;
U represents CH, CH₂, N, C═O, C═S, S═O, SO₂ or NR², under the proviso that, G and U do not simultaneously represent CH₂, and when G and U each represent CH or N, a bond between G and U is a double bond;
R¹ and R² each independently represent hydrogen, cyano, C₁₋₁₂ alkyl, C₃₋₈ cycloalkyl, C₃₋₈ cycloalkyl-C₁₋₁₂ alkyl, C₂₋₆ alkenyl, C₂₋₆ alkynyl, C₁₋₁₂ alkoxy-carbonyl or C₁₋₁₂ thioalkoxy-carbonyl, wherein each group from the C₁₋₁₂ alkyl to the C₁₋₁₂ thioalkoxy-carbonyl described herein may be substituted;
R¹ and R² may form a 3-to 6-membered hydrocarbon ring with the carbon atom to which they are bound;
X which can be the same or different represents hydrogen, halogen, nitro, cyano, hydroxy, mercapto, SF₅, amino, C₁₋₁₂ alkyl, C₁₋₁₂ alkoxy, C₃₋₈ cycloalkyl, C₁₋₁₂ alkylthio, C₁₋₁₂ alkylsulfinyl, C₁₋₁₂ alkylsulfonyl, C₁₋₁₂ alkylsulfonyloxy, C₁₋₁₂ alkylaminosulfonyl, di(C₁₋₁₂ alkyl)aminosulfonyl, C₁₋₁₂ alkylcarbonylamino, benzoylamino, tri(C₁₋₁₂ alkyl)silyl, or C₁₋₁₂ alkoxyimino, C₁₋₁₂ alkylsulfinylimino, C₁₋₁₂ alkylsulfonylimino, C₁₋₁₂alkoxy-carbonyl, C₁₋₁₂ alkylcarbonyl, aminocarbonyl, C₁₋₁₂ alkylamino-carbonyl, amino-thiocarbonyl, C₁₋₁₂ alkylamino-thiocarbonyl, di(C₁₋₁₂ alkyl)amino-carbonyl or di(C₁₋₁₂ alkyl)amino-thiocarbonyl, wherein each group of the said C₁₋₁₂ alkyl to di(C₁₋₁₂ alkyl)amino-thiocarbonyl may be substituted;
m represents 1, 2, 3 or 4; or
the chemical grouping (X)ₘ-Q- stands for a 6-membered aromatic cyclic group having the following formula 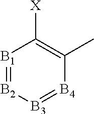
with B₁, B₂, B₃ and B₄ each independently representing C—X or N (nitrogen), under the proviso that only two of B₁, B₂, B₃ and B₄ may simultaneously represent N;
Q represents a 5-membered heterocyclic group which contains 1 to 4 heteroatoms selected from N, O and S;
and wherein the chemical grouping 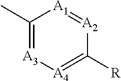
stands for one of the following groups [g1] to [g6] 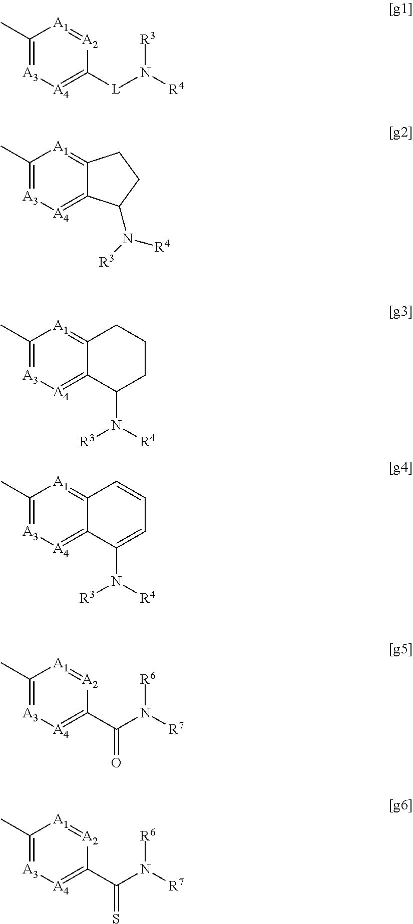
wherein
A₁, A₂, A₃ and A₄ each independently represent C—Y or N (nitrogen) under the proviso that only two of A₁, A₂, A₃ and A₄ may simultaneously represent N; when A₁ and A₂ represent C—Y, two Ys may form a benzene ring or a 5-to 6-membered heteroaromatic ring with the carbon atoms to which they are bound;
L represents a chemical group (CR¹R²)ₙ with
n being 1, 2 or 3;
R³ represents hydrogen, amino, hydroxy, cyano, C₁₋₁₂ alkyl, C₁₋₁₂ alkoxy, C₁₋₁₂ alkylcarbonylamino, C₁₋₁₂ alkylamino, C₃₋₈ cyclo alkyl, C₂₋₁₂ alkenyl, C₂₋₁₂ alkynyl, C₁₋₁₂ alkylcarbonyl, CH₂—R⁵, C(═O)R⁵ or C(═S)R⁵, wherein each group from the C₁₋₁₂ alkyl to the C₁₋₁₂ alkylcarbonyl described herein may be substituted;
R⁴ represents hydrogen, cyano, formyl, thioformyl, C₁₋₁₂ alkylcarbonyl, C₁₋₁₂ alkyl-thiocarbonyl, C₁₋₁₂ alkylamino-carbonyl, C₁₋₁₂ alkylamino-thiocarbonyl, C₂₋₂₄ (total carbon number) dialkylamino-carbonyl, C₂₋₂₄ (total carbon number) dialkylamino-thiocarbonyl, C₁₋₁₂ alkoxyamino-carbonyl, C₁₋₁₂ alkoxyamino-thiocarbonyl, C₁₋₁₂ alkoxy-carbonyl, C₁₋₁₂ alkoxy-C₁₋₁₂ alkylcarbonyl, C₁₋₁₂ thioalkoxy-C₁₋₁₂ alkylcarbonyl, C₁₋₁₂ alkylsulfenyl-C₁₋₁₂ alkylcarbonyl, C₁₋₁₂ alkylsulfonyl-C₁₋₁₂ alkylcarbonyl, C₁₋₁₂ alkoxy-thiocarbonyl, C₁₋₁₂ thioalkoxy-carbonyl, C₁₋₁₂ thio alkoxy-thiocarbonyl, C₁₋₁₂ alkylsulfonyl, C₃₋₈ cycloalkylcarbonyl, C₃₋₈ cycloalkyl-C₁₋₁₂ alkylcarbonyl, C₂₋₁₂ alkenyl-carbonyl, C₂₋₁₂ alkynyl-carbonyl, C₃₋₈ cycloalkylamino-carbonyl, C₂₋₁₂ alkenylamino-carbonyl, C₂₋₁₂ alkynylamino-carbonyl, C(═O)R⁵ or C(═S)R⁵, wherein each group from the C₁₋₁₂ alkylcarbonyl to the C₂₋₁₂ alkynylamino-carbonyl described herein may be substituted; or
R³ and R⁴ may form a 3-to 6-membered heterocycle with the nitrogen atom to which they are bound, wherein the heterocycle may be substituted with X, keto, thioketo or nitroimino;
R⁵ represents phenyl which may be substituted or a 5-to 6-membered heterocyclic group which may be substituted and contains at least one heteroatom selected from N, O and S;
R⁶ and R⁷ each independently represent hydrogen, cyano, C₁₋₁₂ alkyl, C₃₋₈ cycloalkyl, C₂₋₆ alkenyl, C₂₋₆ alkynyl, C₁₋₁₂ alkoxy-carbonyl, C₁₋₁₂ alkylsulfonyl, C₆₋₁₀ aryl, C₇₋₉ aralkyl, a 5-to 6-membered heterocyclic group which contains at least one heteroatom selected from N, O and S, or C₁₋₁₂ alkyl-O—N═CH—, wherein each group from the C₁₋₁₂ alkyl to the C₁₋₁₂ alkyl-O—N═CH— described herein may be substituted; or
R⁶ and R⁷ together may form C₂₋₆ alkylene; preferably R⁶ and R⁷ together may form C₄₋₅ alkylene;
Y which can be the same or different represents hydrogen, halogen, nitro, cyano, hydroxy, mercapto, amino, C₁₋₁₂ alkyl, C₃₋₈ cycloalkyl, C₁₋₁₂ alkoxy, C₁₋₁₂ alkylthio, C₁₋₁₂ alkylsulfinyl, C₁₋₁₂ alkylsulfonyl, C₁₋₁₂ alkylsulfonyloxy, C₁₋₁₂ alkyl amino sulfonyl, C₂₋₂₄ (total carbon number) dialkylamino-sulfonyl, C₁₋₆ alkylcarbonylamino, benzoylamino, tri-C₁₋₁₂ alkylsilyl, C₁₋₁₂ alkoxyimino, C₁₋₁₂ alkylsulfonylimino, C₁₋₁₂ alkylsulfonylimino, C₁₋₁₂ alkoxy-carbonyl, C₁₋₁₂ alkylcarbonyl, aminocarbonyl, C₁₋₁₂ alkylamino-carbonyl, amino-thiocarbonyl, C₁₋₁₂ alkylamino-thiocarbonyl, C₂₋₂₄ (total carbon number) dialkylamino-carbonyl or C₂₋₂₄ (total carbon number) dialkylamino-thiocarbonyl, wherein each group from the amino to the C₂₋₂₄ (total carbon number) dialkylamino-thiocarbonyl described herein may be substituted.

2. Compounds according to claim 1, wherein the chemical grouping 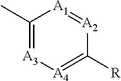
stands for the grouping [g1], and
the chemical grouping (X)ₘ-Q- stands for a 6-membered aromatic cyclic group having the following formula 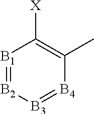
with B₁, B₂, B₃ and B₄ each independently stand for C—X.

3. Compounds according to claim 1, wherein the chemical grouping 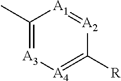
stands for the grouping [g2] or [g3] and the chemical grouping (X)ₘ-Q- stands for a 6-membered aromatic cyclic group having the following formula 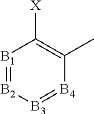
with B₁, B₂, B₃ and B₄ each independently stand for C—X.

4. Compounds according to claim 1 having the formula (I-I) or (I-II) 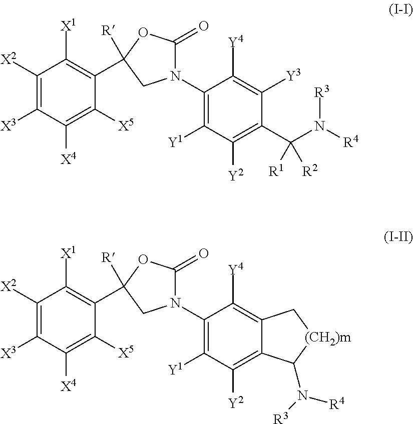
wherein
R′ represents C₁₋₁₂ alkyl or C₁₋₁₂ haloalkyl;
X¹, X², X³, X⁴, X⁵, Y¹, Y², Y³and Y⁴ each independently represent hydrogen, halogen, nitro, cyano, hydroxy, mercapto, amino, SF₅, C₁₋₁₂ alkyl, C₃₋₈ cycloalkyl, C₁₋₁₂ alkoxy, C₁₋₁₂ alkylthio, C₁₋₁₂ alkylsulfinyl, C₁₋₁₂ alkylsulfonyl, C₁₋₁₂ alkylsulfonyloxy, C₁₋₁₂ alkylaminosulfonyl, di(C₁₋₁₂ alkyl)amino-sulfonyl, C₁₋₁₂ alkyl-carbonylamino, benzoylamino, tri(C₁₋₁₂ alkyl)silyl, C₁₋₁₂ alkoxyimino, C₁₋₁₂ alkylsulfonylimino, C₁₋₁₂ alkylsulfonylimino, C₁₋₁₂ alkoxy-carbonyl, C₁₋₁₂ alkyl-carbonyl, aminocarbonyl, C₁₋₁₂ alkylamino-carbonyl, aminothiocarbonyl, C₁₋₁₂ alkylamino-thiocarbonyl, di(C₁₋₁₂ alkyl)amino-carbonyl or di(C₁₋₁₂ alkyl)amino-thiocarbonyl, wherein each group of the said C₁₋₁₂ alkyl to di(C₁₋₁₂ alkyl)amino-thiocarbonyl may be substituted with halogen;
R¹ and R² each independently represent hydrogen, cyano, C₁₋₁₂ alkyl, C₃₋₈ cycloalkyl, C₃₋₈ cycloalkyl-C₁₋₁₂ alkyl, C₂₋₁₂ alkenyl, C₂₋₁₂ alkynyl, C₁₋₁₂ alkoxy-carbonyl or C₁₋₁₂ thioalkoxy-carbonyl, wherein each group of the said C₁₋₁₂ alkyl to C₁₋₁₂ thioalkoxy-carbonyl may be substituted with halogen;
R³ represents hydrogen, amino, hydroxy, cyano, C₁₋₁₂ alkyl, C₁₋₁₂ alkoxy, C₁₋₁₂ alkyl-carbonylamino, C₁₋₁₂ alkylamino, C₃₋₈ cycloalkyl, C₂₋₁₂ alkenyl, C₂₋₁₂ alkynyl, C₁₋₁₂ alkyl-carbonyl, —CH₂—R⁵, —C(═O)R⁵ or —C(═S)R⁵, wherein each group of the said C₁₋₁₂ alkyl to C₁₋₁₂ alkyl-carbonyl may be substituted with halogen;
R⁴ represents hydrogen, cyano, formyl, thioformyl, C₁₋₁₂ alkyl-carbonyl, C₁₋₁₂ alkyl-thiocarbonyl, C₁₋₁₂ alkylamino-carbonyl, C₁₋₁₂ alkylamino-thiocarbonyl, di(C₁₋₁₂ alkyl)amino-carbonyl, di(C₁₋₁₂ alkyl)amino-thiocarbonyl, C₁₋₁₂ alkoxyamino-carbonyl, C₁₋₁₂ alkoxyamino-thiocarbonyl, C₁₋₁₂ alkoxy-carbonyl, C₁₋₁₂ alkoxy-C₁₋₁₂ alkyl-carbonyl, C₁₋₁₂ thioalkoxy-C₁₋₁₂ alkyl-carbonyl, C₁₋₁₂ alkylsulfonyl-C₁₋₁₂ alkyl-carbonyl, C₁₋₁₂ alkylsulfonyl-C₁₋₁₂ alkyl-carbonyl, C₁₋₁₂ alkoxy-thiocarbonyl, C₁₋₁₂ thioalkoxy-carbonyl, C₁₋₁₂ thioalkoxy-thiocarbonyl, C₁₋₁₂ alkylsulfonyl, C₃₋₈ cycloalkyl-carbonyl, C₃₋₈ cycloalkyl-C₁₋₁₂ alkyl-carbonyl, C₂₋₁₂ alkenyl-carbonyl, C₂₋₁₂ alkynyl-carbonyl, C₃₋₈ cycloalkylamino-carbonyl, C₂₋₁₂ alkenylamino-carbonyl, C₂₋₁₂ alkynylamino-carbonyl, —C(═O)R⁵ or —C(═S)R⁵, wherein each group of the said C₁₋₁₂ alkyl-carbonyl to C₂₋₁₂ alkynylamino-carbonyl may be substituted with halogen; and
R⁵ is phenyl which may be substituted, or a 5-or 6-membered heterocycle which contains at least one heteroatom that may be selected from N, O and S and which may be substituted.

5. Compounds according to claim 4, wherein
R′ represents C₁₋₆ alkyl or C₁₋₆ haloalkyl;)
X¹, X², X³, X⁴, X⁵, Y¹, Y², Y³ and Y⁴ each independently represent hydrogen, halogen, nitro, cyano, hydroxy, mercapto, amino, SF_{S}, C₁₋₆ alkyl, C₃₋₇ cycloalkyl, C₁₋₆ alkoxy, C₁₋₆ alkylthio, C₁₋₆ alkylsulfinyl, C₁₋₆ alkylsulfonyl, C₁₋₆ alkylsulfonyloxy, C₁₋₆ alkylaminosulfonyl, di(C₁₋₆ alkyl)amino-sulfonyl, C₁₋₆ alkyl-carbonylamino, benzoylamino, tri(C₁₋₆ alkyl)silyl, C₁₋₆ alkoxyimino, C₁₋₆ alkylsulfinylimino, C₁₋₆ alkylsulfonylimino, C₁₋₆ alkoxy-carbonyl, C₁₋₆ alkyl-carbonyl, aminocarbonyl, C₁₋₆ alkylamino-carbonyl, aminothiocarbonyl, C₁₋₆ alkylamino-thiocarbonyl, di(C₁₋₆ alkyl)amino-carbonyl or di(C₁₋₆ alkyl)amino-thiocarbonyl, wherein each group of the said C₁₋₆ alkyl to di(C₁₋₆ alkyl)amino-thiocarbonyl may be substituted with halogen;
R¹ and R² each independently represent hydrogen, cyano, C₁₋₆ alkyl, C₃₋₇ cycloalkyl, C₃₋₇ cycloalkyl-C₁₋₆ alkyl, C₂₋₆ alkenyl, C₂₋₆ alkynyl, C₁₋₆ alkoxy-carbonyl or C₁₋₆ thioalkoxy-carbonyl, wherein each group of the said C₁₋₆ alkyl to C₁₋₆ thioalkoxy-carbonyl may be substituted with halogen;
R³ represents hydrogen, amino, hydroxy, cyano, C₁₋₆ alkyl, C₁₋₆ alkoxy, C₁₋₆ alkyl-carbonylamino, C₁₋₆ alkylamino, C₃₋₇ cycloalkyl, C₂₋₆ alkenyl, C₂₋₆ alkynyl, C₁₋₆ alkyl-carbonyl, —CH₂—R⁵, —C(═O)R⁵ or —C(═S)R⁵, wherein each group of the said C₁₋₆ alkyl to C₁₋₆ alkyl-carbonyl may be substituted with halogen;
R⁴ represents hydrogen, cyano, formyl, thioformyl, C₁₋₆ alkyl-carbonyl, C₁₋₆ alkyl-thiocarbonyl, C₁₋₆ alkylamino-carbonyl, C₁₋₆ alkylamino-thiocarbonyl, di(C₁₋₆ alkyl)amino-carbonyl, di(C₁₋₆ alkyl)amino-thiocarbonyl, C₁₋₆ alkoxyamino-carbonyl, C₁₋₆ alkoxyamino-thiocarbonyl, C₁₋₆ alkoxy-carbonyl, C₁₋₆ alkoxy-C₁₋₆ alkyl-carbonyl, C₁₋₆ thioalkoxy-C₁₋₆ alkyl-carbonyl, C₁₋₆ alkylsulfonyl-C₁₋₆ alkyl-carbonyl, C₁₋₆ alkylsulfonyl-C₁₋₆ alkyl-carbonyl, C₁₋₆ alkoxy-thiocarbonyl, C₁₋₆ thioalkoxy-carbonyl, C₁₋₆ thioalkoxy-thiocarbonyl, C₁₋₆ alkylsulfonyl, C₃₋₇ cycloalkyl-carbonyl, C₃₋₇ cycloalkyl-C₁₋₆ alkyl-carbonyl, C₂₋₆ alkenyl-carbonyl, C₂₋₆ alkynyl-carbonyl, C₃₋₇ cycloalkylamino-carbonyl, C₂₋₆ alkenylamino-carbonyl, C₂₋₆ alkynylamino-carbonyl, —C(═O)R⁵ or —C(═S)R⁵, wherein each group of the said C₁₋₆ alkyl-carbonyl to C₂₋₆ alkynylamino-carbonyl may be substituted with halogen; and
R⁵ is phenyl which may be substituted, or an optionally substituted 5-or 6-membered heterocycle which contains at least one heteroatom that may be selected from N, O and S.

6. Compounds according to claim 4, in which
R¹ represents C₁₋₄ alkyl or C₁₋₄ haloalkyl; X¹, X², X³, X⁴, X⁵, Y¹, Y², Y³ and Y⁴ each independently represent hydrogen, halogen, nitro, cyano, hydroxy, mercapto, amino, SF_{S}, C₁₋₄ alkyl, C₃₋₆ cycloalkyl, C₁₋₄ alkoxy, C₁₋₄ alkylthio, C₁₋₄ alkylsulfinyl, C₁₋₄ alkylsulfonyl, C₁₋₄ alkylsulfonyloxy, C₁₋₄ alkylaminosulfonyl, di(C₁₋₄ alkyl)amino-sulfonyl, C₁₋₄ alkyl-carbonylamino, benzoylamino, tri(C₁₋₄ alkyl)silyl, C₁₋₄ alkoxyimino, C₁₋₄ alkylsulfinylimino, C₁₋₄ alkylsulfonylimino, C₁₋₄ alkoxy-carbonyl, C₁₋₄ alkyl-carbonyl, aminocarbonyl, C₁₋₄ alkylamino-carbonyl, aminothiocarbonyl, C₁₋₄ alkylamino-thiocarbonyl, di(C₁₋₄ alkyl)amino-carbonyl or di(C₁₋₄ alkyl)amino-thiocarbonyl, wherein each group of the said C₁₋₄ alkyl to di(C₁₋₄ alkyl)amino-thiocarbonyl may be substituted with halogen;
R¹ and R² each independently represent hydrogen, cyano, C₁₋₄ alkyl, C₃₋₆ cycloalkyl, C₃₋₆ cycloalkyl-C₁₋₄ alkyl, C₂₋₄ alkenyl, C₂₋₄ alkynyl, C₁₋₄ alkoxy-carbonyl or C₁₋₄ thioalkoxy-carbonyl, wherein each group of the said C₁₋₄ alkyl to C₁₋₄ thioalkoxy-carbonyl may be substituted with halogen;
R³ represents hydrogen, amino, hydroxy, cyano, C₁₋₄ alkyl, C₁₋₄ alkoxy, C₁₋₄ alkyl-carbonylamino, C₁₋₄ alkylamino, C₃₋₆ cycloalkyl, C₂₋₄ alkenyl, C₂₋₄ alkynyl, C₁₋₄ alkyl-carbonyl, —CH₂—R⁵, —C(═O)R⁵ or —C(═S)R⁵, wherein each group of the said C₁₋₄ alkyl to C₁₋₄ alkyl-carbonyl may be substituted with halogen;
R⁴ represents hydrogen, cyano, formyl, thioformyl, C₁₋₄ alkyl-carbonyl, C₁₋₄ alkyl-thiocarbonyl, C₁₋₄ alkylamino-carbonyl, C₁₋₄ alkylamino-thiocarbonyl, di(C₁₋₄ alkyl)amino-carbonyl, di(C₁₋₄ alkyl)amino-thiocarbonyl, C₁₋₄ alkoxyamino-carbonyl, C₁₋₄ alkoxyamino-thiocarbonyl, C₁₋₄ alkoxy-carbonyl, C₁₋₄ alkoxy-C₁₋₄ alkyl-carbonyl, C₁₋₄ thioalkoxy-C₁₋₄ alkyl-carbonyl, C₁₋₄ alkylsulfenyl-C₁₋₄ alkyl-carbonyl, C₁₋₄ alkylsulfonyl-C₁₋₄ alkyl-carbonyl, C₁₋₄ alkoxy-thiocarbonyl, C₁₋₄ thioalkoxy-carbonyl, C₁₋₄ thioalkoxy-thiocarbonyl, C₁₋₄ alkylsulfonyl, C₃₋₆ cycloalkyl-carbonyl, C₃₋₆ cycloalkyl-C₁₋₄ alkyl-carbonyl, C₂₋₄ alkenyl-carbonyl, C₂₋₄ alkynyl-carbonyl, C₃₋₆ cycloalkylamino-carbonyl, C₂₋₄ alkenylamino-carbonyl, C₂₋₄ alkynylamino-carbonyl, —C(═O)R⁵ or —C(═S)R⁵, wherein each group of the said C₁₋₄ alkyl-carbonyl to C₂₋₄ alkynylamino-carbonyl may be substituted with halogen; and
R⁵ is phenyl which may be substituted, or an optionally substituted 5-or 6-membered heterocycle which contains at least one heteroatom that may be selected from N, O and S.

7. Compounds according to claim 1, having the formula (I-Ia), 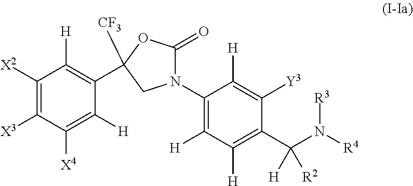
wherein
X², X³ and X⁴ each independently represent hydrogen, halogen or C₁₋₄ haloalkyl;
Y³ represents halogen, C₁₋₄ alkyl or C₁₋₄ haloalkyl;
R² represents hydrogen or C₁₋₄ alkyl;
R³ represents hydrogen or C₁₋₄ alkyl; and
R⁴ represents hydrogen, C₁₋₄ alkyl-carbonyl, C₁₋₄ haloalkyl-carbonyl, C₃₋₆ cycloalkyl-carbonyl, C₁₋₄ alkoxy-C₁₋₄ alkyl-carbonyl, C₁₋₄ thioalkoxy-C₁₋₄ alkyl-carbonyl, C₁₋₄ alkylsulfenyl-C₁₋₄ alkyl-carbonyl, C₁₋₄ alkylsulfonyl-C₁₋₄ alkyl-carbonyl or C₁₋₄ alkylamino-carbonyl.

8. A pesticidal agent which contains, as an effective component, the compound according to claim 4.

9. An animal parasite-controlling agent which contains, as an effective component, the compound according to claim 4.

10. A method for combating harmful agricultural pests, comprising applying the compound of claim 1 to the pests.

11. The method according to claim 10, wherein the harmful agricultural pests are selected from the group consisting of insects, acari and nematodes which attack plants or plant parts.

12. A method for combating endo- or ectoparasites on an animal or a human, comprising treating the animal or human with the compound of claim 1.

13. A compound combination, comprising a compound according to claim 1 and at least one active compound selected from the group consisting of insecticides, acaricides, nematicides, funizides and microbiologica.

14. An intermediate for the manufacturing of a compound according to claim 1 having the formula (r-3) or (r-4) 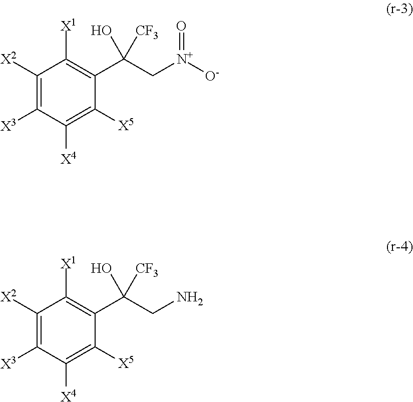
wherein
X¹, X², X³, X⁴ and X⁵ are as defined for X in claim 1.

15. An intermediate for the manufacturing of a compound according to any one of claim 1 having the formula (IIb) 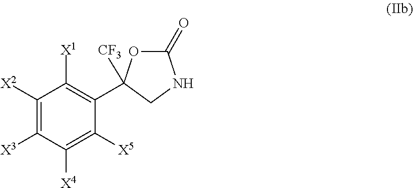
wherein
X¹, X², X³, X⁴ and X⁵ are as defined for X in claim 1.

16. An intermediate for the manufacturing of a compound according to claim 1 having the formula (r-6) 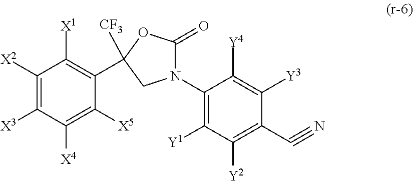
wherein
X¹, X², X³, X⁴ and X⁵ are as defined for X in claim 1, and Y¹, Y², Y³, and Y⁴ are as defined for Y in claim 1.
